(19) United States
(12) Patent Application Publication
(10) Pub. No.: US 2023/0083005 A1
(43) Pub. Date: Mar. 16, 2023

(54) METHODS OF KILLING OR INHIBITING THE GROWTH OF CANCER CELLS

(71) Applicant: TISSUETECH, INC., Miami, FL (US)

(72) Inventors: Scheffer TSENG, Pinecrest, FL (US); Hua HE, Miami, FL (US); Sean TIGHE, Tampa, FL (US); Kaustuv BASU, Miami, FL (US)

(21) Appl. No.: 17/798,421

(22) PCT Filed: Feb. 12, 2021
(86) PCT No.: PCT/US2021/017773

§ 371 Date: Aug. 9, 2022

Related U.S. Application Data
(60) Provisional application No. 62/975,599, filed on Feb. 12, 2020.

(51) Int. Cl. A61K 47/62; A61K 47/61; A61K 45/06; A61P 35/00; A61P 13/08

(52) U.S. Cl. CPC A61K 47/62; A61K 47/61; A61K 45/06; A61P 35/00; A61P 13/08

(57) ABSTRACT

Provided herein, in certain embodiments, are compositions comprising an isolated heavy chain-hyaluronan/pentraxin 3 (“HC-HA/PTX3”) complex for use in methods of killing cancer cells. Also provided herein, are methods of inhibiting cancer cell regrowth of a tumor in an individual in need thereof, comprising contacting an area surrounding the tumor after a surgical procedure with an isolated HC-HA/PTX3 complex. Combinations and kits for use in practicing said methods also are provided herein.

### CROSS REFERENCE

The present application claims priority to and benefit from U.S. Provisional Application No. 62/975,599, filed on Feb. 12, 2020, the entire contents of which are herein incorporated by reference.

### SUMMARY OF THE INVENTION

All publications, patents, and patent applications mentioned in this specification are herein incorporated by reference to the same extent as if each individual publication, patent, or patent application was specifically and individually indicated to be incorporated by reference.

Disclosed herein are methods of directly killing cancer cells in a subject in need thereof, comprising contacting the cancer cells with an isolated HC-HA/PTX3 complex. In some embodiments, the cancer cells are from or within a solid tumor. In some embodiments, the cancer cells are from a cancer selected from the group consisting of liver cancer, pancreatic cancer, bladder cancer, prostate cancer, lung cancer, non-small cell lung carcinoma, ovarian cancer, breast cancer, melanoma, gastric cancer, colon cancer, colorectal cancer, central nervous system (CNS) cancer, bone cancer, a lymphoma, skin cancer, head and neck cancer, kidney cancer, testicular cancer, uterine cancer, cervical cancer, esophageal cancer, thyroid cancer, salivary gland cancer, adrenal cancer, and gastrointestinal cancer. In some embodiments, the cancer is inoperable. In some embodiments, the CNS cancer is a glioma or a metastatic cancer. In some embodiments, the glioma is glioblastoma multiforme or anaplastic astrocytoma. In some embodiments, the cancer is lung cancer, breast cancer, colon cancer or skin cancer. In some embodiments, the colon cancer is adenocarcinoma. In some embodiments, the skin cancer is melanoma. In some embodiments, the cancer is prostate cancer. In some embodiments, the contacting comprises injecting the HC-HA/PTX3 complex into the tumor, the surrounding tissue, or a combination thereof. In some embodiments, the contacting is prior to, during, or after surgical excision, cryoablation, or radiofrequency ablation of the cancer cells. In some embodiments, the contacting comprises applying the HC-HA/PTX3 to surgical margins from the surgical excision of the cancer cells, or into any remaining portion of tumor. In some embodiments, the isolated HC-HA/PTX3 complex is native HC-HA/PTX3 complex, reconstituted HC-HA/PTX3 complex, or a combination thereof. In some embodiments, the native HC-HA/PTX3 complex is isolated from a fetal support tissue. In some embodiments, the reconstituted HC-HA/PTX3 complex comprises heavy chain 1 (HC1) and heavy chain 2 (HC2) of inter-α-inhibitor (IαI), hyaluronic acid (HA), and PTX3. In some embodiments, the reconstituted HC-HA/PTX3 complex comprises HC1, HC2, HA, PTX3 and TSG-6. In some embodiments, the HC-HA/PTX3 is comprised in a composition comprising a pharmaceutically acceptable pharmaceutically acceptable diluent, excipient, vehicle, or carrier. In some embodiments, the method further comprising administering a further therapeutic agent. In some embodiments, the therapeutic agent is selected from the group consisting of a chemotherapeutic, an analgesic, an anti-inflammatory, a steroid, an immunotherapy, cellular therapy, a radiotherapy, a targeted drug therapeutic, and an antibiotic. In some embodiments, administering the therapeutic agent occurs before contacting the cancer cells with the HC-HA/PTX3 complex. In some embodiments, administering the therapeutic agent occurs after contacting the cancer cells with the HC-HA/PTX3 complex. In some embodiments, administering the therapeutic agent occurs concurrently with contacting the cancer cells with the HC-HA/PTX3 complex. In some embodiments, angiogenesis is reduced or inhibited. In some embodiments, killing cancer cells is via apoptosis or necrosis.

Disclosed herein are methods of directly inhibiting proliferation of cancer cells in a subject in need thereof, comprising contacting the cancer cells with an isolated HC-HA/PTX3 complex. In some embodiments, the cancer cells are from or within a solid tumor. In some embodiments, the cancer cells are from a cancer selected from the group consisting of liver cancer, pancreatic cancer, bladder cancer, prostate cancer, lung cancer, non-small cell lung carcinoma, ovarian cancer, breast cancer, melanoma, gastric cancer, colon cancer, colorectal cancer, central nervous system (CNS) cancer, bone cancer, a lymphoma, skin cancer, head and neck cancer, kidney cancer, testicular cancer, uterine cancer, cervical cancer, esophageal cancer, thyroid cancer, salivary gland cancer, adrenal cancer, and gastrointestinal cancer. In some embodiments, the cancer is inoperable. In some embodiments, the CNS cancer is a glioma or a metastatic cancer. In some embodiments, the glioma is glioblastoma multiforme or anaplastic astrocytoma. In some embodiments, the cancer is lung cancer, breast cancer, colon cancer or skin cancer. In some embodiments, the colon cancer is adenocarcinoma. In some embodiments, the skin cancer is melanoma. In some embodiments, the cancer is prostate cancer. In some embodiments, the contacting comprises injecting the HC-HA/PTX3 complex into the tumor, the surrounding tissue, or a combination thereof. In some embodiments, the contacting is prior to, during, or after surgical excision, cryoablation, or radiofrequency ablation of the cancer cells. In some embodiments, the contacting comprises applying the HC-HA/PTX3 to surgical margins from the surgical excision of the cancer cells. In some embodiments, the isolated HC-HA/PTX3 complex is native HC-HA/PTX3 complex, reconstituted HC-HA/PTX3 complex, or a combination thereof. In some embodiments, the native HC-HA/PTX3 complex is isolated from a fetal support tissue. In some embodiments, the reconstituted HC-HA/PTX3 complex comprises heavy chain 1 (HC1) and heavy chain 2 (HC2) of inter-α-inhibitor (IαI), hyaluronic acid (HA), and PTX3. In some embodiments, the reconstituted HC-HA/PTX3 complex comprises HC1, HC2, HA, PTX3 and TSG-6. In some embodiments, comprising a pharmaceutically acceptable diluent, excipient, vehicle, or carrier. In further embodiments, the HC-HA/PTX3 is comprised in a composition comprising administering a therapeutic agent. In some embodiments, the therapeutic agent is selected from the group consisting of a chemotherapeutic, an analgesic, an anti-inflammatory, a steroid, an immunotherapy, cellular therapy, a radiotherapy, a targeted drug therapeutic, and an antibiotic. In some embodiments, administering the therapeutic agent occurs before contacting the cancer cells with the HC-HA/PTX3 complex. In some embodiments, administering the therapeutic agent occurs after contacting the cancer cells with the HC-HA/PTX3 complex. In some embodiments, administering the therapeutic agent occurs concurrently with contacting the cancer cells with the HC-HA/PTX3 complex. In some embodiments, angiogenesis is reduced or inhibited. In some embodiments, proliferation is inhibited in cells expressing CD44 or RHAMM.

Described herein are methods of directly reducing metabolic activity of cancer cells in a subject in need thereof, comprising contacting the cancer cells with an isolated HC-HA/PTX3 complex. In some embodiments, the cancer cells are from or within a solid tumor. In some embodiments, the cancer cells are from a cancer selected from the group consisting of liver cancer, pancreatic cancer, bladder cancer, prostate cancer, lung cancer, non-small cell lung carcinoma, ovarian cancer, breast cancer, melanoma, gastric cancer, colon cancer, colorectal cancer, central nervous system (CNS) cancer, bone cancer, a lymphoma, skin cancer, head and neck cancer, kidney cancer, testicular cancer, uterine cancer, cervical cancer, esophageal cancer, thyroid cancer, salivary gland cancer, adrenal cancer, and gastrointestinal cancer. In some embodiments, the cancer is inoperable. In some embodiments, the CNS cancer is a glioma or a metastatic cancer. In some embodiments, the glioma is glioblastoma multiforme or anaplastic astrocytoma. In some embodiments, the cancer is lung cancer, breast cancer, colon cancer or skin cancer. In some embodiments, the colon cancer is adenocarcinoma. In some embodiments, the skin cancer is melanoma. In some embodiments, the cancer is prostate cancer. In some embodiments, the contacting comprises injecting the HC-HA/PTX3 complex into the tumor, the surrounding tissue, or a combination thereof. In some embodiments, the contacting is prior to, during, or after surgical excision, cryoablation, or radiofrequency ablation of the cancer cells, or into any remaining portion of tumor. In some embodiments, the contacting comprises applying the HC-HA/PTX3 to surgical margins from the surgical excision of the cancer cells. In some embodiments, the isolated HC-HA/PTX3 complex is native HC-HA/PTX3 complex, reconstituted HC-HA/PTX3 complex, or a combination thereof. In some embodiments, the native HC-HA/PTX3 complex is isolated from a fetal support tissue. In some embodiments, the reconstituted HC-HA/PTX3 complex comprises heavy chain 1 (HC1) and heavy chain 2 (HC2) of inter-α-inhibitor (IαI), hyaluronic acid (HA), and PTX3. In some embodiments, the reconstituted HC-HA/PTX3 complex comprises HC1, HC2, HA, PTX3 and TSG-6. In some embodiments, the HC-HA/PTX3 is comprised in a composition comprising a pharmaceutically acceptable pharmaceutically acceptable diluent, excipient, vehicle, or carrier. In further embodiments, the HC-HA/PTX3 is comprised in a composition comprising administering a therapeutic agent. In some embodiments, the therapeutic agent is selected from the group consisting of a chemotherapeutic, an analgesic, an anti-inflammatory, a steroid, an immunotherapy, cellular therapy, a radiotherapy, a targeted drug therapeutic, and an antibiotic. In some embodiments, administering the therapeutic agent occurs before contacting the cancer cells with the HC-HA/PTX3 complex. In some embodiments, administering the therapeutic agent occurs after contacting the cancer cells with the HC-HA/PTX3 complex. In some embodiments, administering the therapeutic agent occurs concurrently with contacting the cancer cells with the HC-HA/PTX3 complex. In some embodiments, angiogenesis is reduced or inhibited. In some embodiments, metabolic activity is reduced in cells expressing CD44 or RHAMM.

Described herein are methods of killing cancer cells, comprising contacting surgical margins or any portion of a tumor prior to, during, or after surgical excision, cryoablation, or radiofrequency ablation of a tumor with isolated HC-HA/PTX3 complex, thereby killing cancer cells in the surgical margins. In some embodiments, the tumor is a cancer selected from the group consisting of liver cancer, pancreatic cancer, bladder cancer, prostate cancer, lung cancer, non-small cell lung carcinoma, ovarian cancer, breast cancer, melanoma, gastric cancer, colon cancer, colorectal cancer, central nervous system (CNS) cancer, bone cancer, a lymphoma, skin cancer, head and neck cancer, kidney cancer, testicular cancer, uterine cancer, cervical cancer, esophageal cancer, thyroid cancer, salivary gland cancer, adrenal cancer, and gastrointestinal cancer. In some embodiments, the cancer is inoperable. In some embodiments, the CNS cancer is a glioma or a metastatic cancer. In some embodiments, the glioma is glioblastoma multiforme. In some embodiments, the cancer is lung cancer, breast cancer, colon cancer or skin cancer. In some embodiments, the colon cancer is adenocarcinoma. In some embodiments, the skin cancer is melanoma. In some embodiments, the tumor is a prostate cancer. In some embodiments, the HC-HA/PTX3 complex is native HC-HA/PTX3 complex, reconstituted HC-HA/PTX3 complex, or a combination thereof. In some embodiments, the native HC-HA/PTX3 complex is isolated from a fetal support tissue. In some embodiments, the reconstituted HC-HA/PTX3 complex comprises heavy chain 1 (HC1) and heavy chain 2 (HC2) of inter-α-inhibitor (IαI), hyaluronic acid (HA), and PTX3. In some embodiments, the reconstituted HC-HA/PTX3 complex comprises HC1, HC2, HA, PTX3 and TSG-6. In some embodiments, the HC-HA/PTX3 is comprised in a composition comprising a pharmaceutically acceptable pharmaceutically acceptable diluent, excipient, vehicle, or carrier. In further embodiments, the reconstituted HC-HA/PTX3 complex comprises administering a therapeutic agent. In some embodiments, the therapeutic agent is selected from the group consisting of a chemotherapeutic, an analgesic, an anti-inflammatory, a steroid, an immunotherapy, cellular therapy, a radiotherapy, a targeted drug therapeutic, and an antibiotic. In some embodiments, administering the therapeutic agent occurs before contacting the cancer cells with the HC-HA/PTX3 complex. In some embodiments, administering the therapeutic agent occurs after contacting the cancer cells with the HC-HA/PTX3 complex. In some embodiments, administering the therapeutic agent occurs concurrently with contacting the cancer cells with the HC-HA/PTX3 complex. In some embodiments, the killing cancer cells is by apoptosis or necrosis.

Disclosed herein, in some embodiments, are methods of inhibiting cancer cell regrowth of a tumor in an individual in need thereof, comprising contacting an area surrounding the tumor after a surgical procedure with an isolated heavy chain-hyaluronan/pentraxin 3 (HC-HA/PTX3) complex, thereby inhibiting cancer cell regrowth at the area surrounding the tumor. In some embodiments, the surgical procedure comprises surgical excision, cryoablation, or radiofrequency ablation of the tumor. In some embodiments, the surgical procedure comprises chemotherapy, immunotherapy, or targeted therapy. In some embodiments, the area surrounding the tumor comprises a surgical margin. In some embodiments, the area surrounding the tumor is a peritumor region. In some embodiments, the tumor is a solid tumor. In some embodiments, the tumor is a cancer selected from the group consisting of liver cancer, pancreatic cancer, bladder cancer, prostate cancer, lung cancer, non-small cell lung carcinoma, ovarian cancer, breast cancer, melanoma, gastric cancer, colon cancer, colorectal cancer, central nervous system (CNS) cancer, bone cancer, a lymphoma, skin cancer, head and neck cancer, kidney cancer, testicular cancer, uterine cancer, cervical cancer, esophageal cancer, thyroid cancer, salivary gland cancer, adrenal cancer, and gastrointestinal cancer. In some embodiments, the cancer is an inoperable cancer. In some embodiments, the cancer is pancreatic cancer. In some embodiments, the cancer is prostate cancer. In some embodiments, the cancer is glioblastoma multiforme. In some embodiments, the cancer is skin cancer. In some embodiments, the cancer is colon cancer. In some embodiments, the cancer is lung cancer. In some embodiments, the cancer is breast cancer. In some embodiments, the area surrounding the tumor is contacted with about 10 microgram to 100 milligrams. In some embodiments, the HC-HA/PTX3 complex is native HC-HA/PTX3 complex, reconstituted HC-HA/PTX3 complex, or a combination thereof. In some embodiments, the native HC-HA/PTX3 complex is isolated from a fetal support tissue. In some embodiments, the reconstituted HC-HA/PTX3 complex comprises heavy chain 1 (HC1) and heavy chain 2 (HC2) of inter-α-inhibitor (IαI), hyaluronic acid (HA), and PTX3. In some embodiments, the reconstituted HC-HA/PTX3 complex comprises HC1, HC2, HA, PTX3 and tumor necrosis factor α-stimulated gene 6 (TSG-6). In some embodiments, the hyaluronan (HA) is high molecular weight hyaluronan (HMW HA). In some embodiments, the hyaluronan (HA) is low molecular weight hyaluronan (LMW HA). In some embodiments, the HC-HA/PTX3 complex is cryopreserved. In some embodiments, the HC-HA/PTX3 complex comprises viable cells. In some embodiments, the method further comprises administering a therapeutic agent. In some embodiments, the therapeutic agent is selected from the group consisting of a chemotherapeutic, an analgesic, an anti-inflammatory, a steroid, an immunotherapy, cellular therapy, a radiotherapy, a targeted drug therapeutic, and an antibiotic. In some embodiments, administering the therapeutic agent occurs before contacting the area surrounding the tumor with the HC-HA/PTX3 complex. In some embodiments, administering the therapeutic agent occurs after contacting the area surrounding the tumor with the HC-HA/PTX3 complex. In some embodiments, administering the therapeutic agent occurs concurrently with contacting the area surrounding the tumor with the HC-HA/PTX3 complex. In some embodiments, the method inhibits tumor cell regrowth by killing cancer cells. In some embodiments, the killing of the cancer cells is by apoptosis or necrosis. In some embodiments, the method inhibits tumor cell regrowth by inhibiting proliferation of cancer cells. In some embodiments, the method inhibits tumor cell regrowth by inhibiting metabolic activity of cancer cells.

Disclosed herein, in some embodiments, are methods of killing cancer cells of a tumor in an individual in need thereof, comprising contacting a tumor or an area surrounding the tumor prior to, during or after a surgical procedure with an isolated heavy chain-hyaluronan/pentraxin 3 (HC-HA/PTX3) complex, thereby killing the cancer cells. In some embodiments, the surgical procedure comprises surgical excision, cryoablation, or radiofrequency ablation of the tumor. In some embodiments, the surgical procedure comprises chemotherapy, immunotherapy, or targeted therapy. In some embodiments, the area surrounding the tumor comprises a surgical margin. In some embodiments, the area surrounding the tumor is a peritumor region. In some embodiments, the tumor is a solid tumor. In some embodiments, the tumor is a cancer selected from the group consisting of liver cancer, pancreatic cancer, bladder cancer, prostate cancer, lung cancer, non-small cell lung carcinoma, ovarian cancer, breast cancer, melanoma, gastric cancer, colon cancer, colorectal cancer, central nervous system (CNS) cancer, bone cancer, a lymphoma, skin cancer, head and neck cancer, kidney cancer, testicular cancer, uterine cancer, cervical cancer, esophageal cancer, thyroid cancer, salivary gland cancer, adrenal cancer, and gastrointestinal cancer. In some embodiments, the cancer is an inoperable cancer. In some embodiments, the cancer is pancreatic cancer. In some embodiments, the cancer is prostate cancer. In some embodiments, the cancer is glioblastoma multiforme. In some embodiments, the cancer is skin cancer. In some embodiments, the cancer is colon cancer. In some embodiments, the cancer is lung cancer. In some embodiments, the cancer is breast cancer. In some embodiments, the area surrounding the tumor is contacted with about 10 microgram to 100 milligrams. In some embodiments, the HC-HA/PTX3 complex is native HC-HA/PTX3 complex, reconstituted HC-HA/PTX3 complex, or a combination thereof. In some embodiments, the native HC-HA/PTX3 complex is isolated from a fetal support tissue. In some embodiments, the reconstituted HC-HA/PTX3 complex comprises heavy chain 1 (HC1) and heavy chain 2 (HC2) of inter-α-inhibitor (IαI), hyaluronic acid (HA), and PTX3. In some embodiments, the reconstituted HC-HA/PTX3 complex comprises HC1, HC2, HA, PTX3 and tumor necrosis factor α-stimulated gene 6 (TSG-6). In some embodiments, the HC-HA/PTX3 complex is cryopreserved. In some embodiments, the HC-HA/PTX3 complex comprises viable cells. In some embodiments, the hyaluronan (HA) is high molecular weight hyaluronan (HMW HA). In some embodiments, the hyaluronan (HA) is low molecular weight hyaluronan (LMW HA). In some embodiments, the contacting comprises injecting the HC-HA/PTX3 directly into a tumor. In some embodiments, the method further comprises administering a therapeutic agent. In some embodiments, the therapeutic agent is selected from the group consisting of a chemotherapeutic, an analgesic, an anti-inflammatory, a steroid, an immunotherapy, cellular therapy, a radiotherapy, a targeted drug therapeutic, and an antibiotic. In some embodiments, administering the therapeutic agent occurs before contacting the cancer cells with the HC-HA/PTX3 complex. In some embodiments, administering the therapeutic agent occurs after contacting the cancer cells with the HC-HA/PTX3 complex. In some embodiments, administering the therapeutic agent occurs concurrently with contacting the cancer cells with the HC-HA/PTX3 complex. In some embodiments, the killing of the cancer cells is by apoptosis or necrosis.

### BRIEF DESCRIPTION OF THE DRAWINGS

FIG. 1A shows aggregation of LNCaP cells after culturing in RPMI medium.

FIG. 1B shows even distribution of PC-3 cells after culturing in RPMI medium.

FIG. 2A-FIG. 2D show morphology and cell metabolic activity of LNCaP with treatment of a series doses of the refined BTGel or HC-HA/PTX3.

FIG. 3A-FIG. 3D show morphology and metabolic activity of PC-3 with treatment of a series doses of the refined BTGel or HC-HA/PTX3.

FIG. 4A shows WST-1 assay data in LNCaP cells after treatment with UC extract, HC-HA/PTX3, and HA using UC extract in water.

FIG. 4B shows WST-1 assay data in PC-3 cells after treatment with UC extract, HC-HA/PTX3, and HA using UC extract in water.

FIG. 5A-FIG. 5C show morphology of LNCaP cells following treatment with HA (FIG. 5A), HC-HA/PTX3 (FIG. 5B), and umbilical cord extract (UCE) (FIG. 5C).

FIG. 6A-FIG. 6C show morphology of PC-3 cells following treatment with HA (FIG. 6A), HC-HA/PTX3 (FIG. 6B), and umbilical cord extract (UCE) (FIG. 6C).

FIG. 7 shows LNCaP cells grown on laminin and collagen type IV exhibit more cell aggregation than cells grown on other surfaces.

FIG. 8A shows bright-field image of PrEC prostate cell line morphology taken in 10× and 20× magnifications.

FIG. 8B shows bright-field image of PNT2 prostate cell line morphology taken in 10× and 20× magnifications.

FIG. 9 shows representative bright-field microscopic images (scale bar 50 μm) of human primary normal prostate cells after 48 h incubation with different concentrations of HC-HA/PTX3 and HMW-HA.

FIG. 10A and 10B shows metabolic activity (%) evaluated in the normal human primary prostate epithelial cells PrEC (FIG. 10A) and normal human prostate cell line PNT2 (FIG. 10B) by WST-1 assay after 48 h incubation with different concentrations (0.78, 3.125, 6.25, 12.5, 25, 50, 100 μg/ml) of HC-HA/PTX3 and HA. P-value calculated by 2-tailed t-test with respect to the untreated samples.

FIG. 11A and 11B show comparative analysis of the metabolic activity (%) (FIG. 11A) and log scale metabolic activity (FIG. 11B) evaluated in the normal primary prostate epithelial cells (PrEC) & cell lines (PNT2) and prostate cancer cell lines: PC3 & LNCaP after 48 h incubation with different concentrations (0.78, 3.125, 6.25, 12.5, 25, 50, 100 μg/ml) of HC-HA/PTX3.

FIG. 12A and 12B show comparative analysis of the metabolic activity (%) (FIG. 12A) and log scale metabolic activity (FIG. 12B) evaluated in the normal primary prostate epithelial cells (PrEC) & cell lines (PNT2) and prostate cancer cell lines: PC3 & LNCaP after 48 h incubation with different concentrations of HA.

FIG. 13 shows representative bright-field microscopic images (scale bar 50 μm) of A375 (melanoma) cells after 48 h incubation with different concentrations of HC-HA/PTX3 and HMW-HA.

FIG. 14 shows representative bright-field microscopic images (scale bar 50 μm) of HT-29 (colon cancer) cells after 48 h incubation with different concentrations of HC-HA/PTX3 and HMW-HA.

FIG. 15 shows representative bright-field microscopic images (scale bar 50 μm) of A549 (lung cancer) cells after 48 h incubation with different concentrations of HC-HA/PTX3 and HMW-HA.

FIG. 16 shows representative bright-field microscopic images (scale bar 50 μm) of MCF-7 (breast cancer) cells after 48 h incubation with different concentrations of HC-HA/PTX3 and HMW-HA.

FIG. 17A-17D show metabolic activity (%) evaluated in 4 human cancer cell lines: A375 (FIG. 17A), HT-29 (FIG. 17B), MCF-7 (FIG. 17C), and A-549 (FIG. 17D) by WST-1 assay after 48 h incubation with different concentrations of HC-HA/PTX3 and HA.

FIG. 18A shows representative bright-field microscopic images (scale bar 50 μm) of LNC (limbal niche cells) after treatment with different concentrations of HC-HA/PTX3 for different time points: 15-30 min, 1 h, 5 h, 24 h and 48 h respectively.

FIG. 18B shows representative bright-field microscopic images (scale bar 50 μm) of LNC (limbal niche cells) after treatment with different concentrations of HMW-HA for different time points: 15-30 min, 1 h, 5 h, 24 h and 48 h respectively.

FIG. 18C shows representative bright-field microscopic image (scale bar 50 μm) of LNC (limbal niche cells) after 48 h incubation with 100 μg/ml of HC-HA/PTX3 and HMW-HA.

FIG. 19 shows metabolic activity (%) evaluated in limbal niche cells by WST-1 assay after 48 h incubation with different concentrations of HC-HA/PTX3 and HA.

FIG. 20A and 20B show representative bright-field microscopic images (scale bar 50 μm) of HTM (human trabecular meshwork) cells after treatment with different concentrations of HC-HA/PTX3 (FIG. 20A) and HMW-HA (FIG. 20B) for different time points.

FIG. 21 shows metabolic activity (%) evaluated in human trabecular meshwork cells by WST-1 assay after 48 h incubation with different concentrations of HC-HA/PTX3 and HA.

FIG. 22A and 22B shows representative brightfield microscopic images (scale bar 50 μm) of human corneal fibroblast (HCF) cells after treated with different concentrations of HC-HA/PTX3 (FIG. 22A) and HMW-HA (FIG. 22B) for different time points.

FIG. 23 shows metabolic activity (%) evaluated in human corneal fibroblast cells by WST-1 assay after 48 h incubation with different concentrations of HC-HA/PTX3 and HA.

FIG. 24A and 24B show comparative analysis of the metabolic activity (%) evaluated in three types of human normal primary mesenchymal cells: HCF, HTM & LNC as evaluated by WST-1 assay after 48 h incubation with different concentrations of HC-HA/PTX3 (FIG. 24A) and HA (FIG. 24B).

FIG. 25 provides representative bright-field microscopic images (scale bar 50 μm) showing transient effect of HC-HA/PTX3 (100 μg/ml) on the morphology of LNC and HCF cells, without a corresponding effect in HTM cells.

FIG. 26A shows bright-field images of A375 cell morphology following treatment with HC-HA/PTX3 and HA, compared to untreated cells.

FIG. 26B shows BrdU cell proliferation assay curve using A375 cells.

FIG. 26C shows semilog scale BrdU cell proliferation assay curve using A375 cells.

FIG. 27A shows bright-field images of PrEC cell morphology in two magnifications (10× & 20×) after treatment with HA or HC-HA/PTX3.

FIG. 27B shows BrdU cell proliferation assay curve following HC-HA/PTX3 treatment.

FIG. 27C shows BrdU cell proliferation assay curve following HA treatment.

FIG. 28A shows brightfield images of PNT2 cell morphology following treatment with HC-HA/PTX3, HMW-HA, or untreated.

FIG. 28B shows BrdU cell proliferation assay curve in PNT2 cells following treatment with HC-HA/PTX3 or HA.

FIG. 29A shows bright-field images of PC3 cell morphology following treatment with HC-HA/PTX3, HMW-HA, or untreated.

FIG. 29B shows BrdU cell proliferation assay curve in PC3 cells following treatment with HC-HA/PTX3 or HA.

FIG. 30A shows bright-field images of LNCaP cell morphology following treatment with HC-HA/PTX3, HMW-HA, or untreated.

FIG. 30B shows BrdU cell proliferation assay curve in LNCaP cells following treatment with HC-HA/PTX3 or HA.

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. All patents, patent applications, published applications and publications, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, ranges and amounts can be expressed as “about” a particular value or range. About also includes the exact amount or value. Hence “about 5 μg” means “about 5 μg” and also “5 μg.” In some embodiments, the term “about” includes an amount that would be expected to be within experimental error. In some embodiments, the term “about” refers to the value +/−20%, 10% or 5% of the value.

As used herein, HC-HA/PTX3 complex or isolated HC-HA/PTX3 refers to native HC-HA/PTX3, reconstituted HC-HA/PTX3 or a combination thereof. As used herein, a reconstituted HC-HA/PTX3 (rcHC-HA/PTX3) complex is an HC-HA/PTX3 complex that is formed by assembly of the component molecules of the complex in vitro. In some embodiments, the rcHC-HA/PTX3 complex comprises HA, HC1 and HC2 of IαI, and PTX3. In some embodiments, the rcHC-HA/PTX3 complex comprises HA, HC1 and HC2 of IαI, PTX3, and TSG-6. The process of assembling the rcHC-HA/PTX3 includes reconstitution with purified native proteins or molecules isolated from biological sources, recombinant proteins generated by recombinant methods, or synthesis of molecules by in vitro synthesis. In some instances, the purified native proteins used for assembly of the rcHC-HA/PTX3 are proteins in a complex with other proteins (i.e. a multimer, a multichain protein or other complex). In some instances, PTX3 is purified as a multimer (e.g. a homomultimer) from a cell and employed for assembly of the rcHC-HA/PTX3 complex.

As used herein, a purified native HC-HA/PTX3 (nHC-HA/PTX3) complex refers to an HC-HA/PTX3 complex that is purified from a biological source such as a cell, a tissue or a biological fluid. In some embodiments, the HC-HA/PTX3 is isolated from a fetal support tissue, such as placenta, amniotic membrane, chorion, umbilical cord, or umbilical cord amniotic membrane. In some embodiments, the HC-HA/PTX3 is isolated from amniotic membrane. In some embodiments, the native HC-HA/PTX3 complex comprises HA, HC1 of IαI, and PTX3. Such complexes are generally assembled in vivo in a subject or ex vivo in cells, tissues, or biological fluids from a subject, including a human or other animal.

As used herein, “hyaluronan,” “hyaluronic acid,” or “hyaluronate” (HA) are used interchangeably to refer to a substantially non-sulfated linear glycosaminoglycan (GAG) with repeating disaccharide units of D-glucuronic acid and N-acetylglucosamine (D-glucuronosyl-N-acetylglucosamine).

As used herein, the term “high molecular weight” or “HMW,” as in high molecular weight hyaluronan (HMW HA), is meant to refer to HA that has a weight average molecular weight that is greater than about 500 kilodaltons (kDa), such as, for example, between about 500 kDa and about 10,000 kDa, between about 800 kDa and about 8,500 kDa, between about 1100 kDa and about 5,000 kDa, or between about 1400 kDa and about 3,500 kDa. In some embodiments, the HMW HA has a weight average molecular weight of 3000 kDa or greater. In some embodiments, the HMW HA has a weight average molecular weight of 3000 kDa. In some embodiments, the HMW HA is Healon® with a weight average molecular weight of about 3000 kDa. In some embodiments, HMW HA has a molecular weight of between about 500 kDa and about 10,000 kDa. In some embodiments, HMW HA has a molecular weight of between about 800 kDa and about 8,500 kDa. In some embodiments, HMW HA has a molecular weight of about 3,000 kDa.

As used herein, the term “low molecular weight” or “LMW,” as in low molecular weight hyaluronan (LMW HA), is meant to refer to HA that has a weight average molecular weight that is less than 500 kDa, such as for example, less than about 400 kDa, less than about 300 kDa, less than about 200 kDa, about 200-300 kDa, or about 1-300 kDa.

As used herein, pentraxin 3, or PTX3, protein or polypeptide refers to any PTX3 protein, including but not limited to, a recombinantly produced protein, a synthetically produced protein, a native PTX3 protein, and a PTX3 protein extracted from cells or tissues. PTX3 include multimeric forms (e.g. homomultimer) of PTX3, including, but not limited to, dimeric, trimeric, tetrameric, pentameric, hexameric, tetrameric, octameric, and other multimeric forms naturally or artificially produced.

As used herein, a “hyaluronan binding protein”, “HA binding protein”, or “HABP” refers to any protein that specifically binds to HA.

As used herein, “link module” means a hyaluronan-binding domains.

As used herein, “biological activity” refers to the in vivo activities of an nHC-HA/PTX3 or rcHC-HA/PTX3 complex or physiological responses that result upon in vivo administration of an nHC-HA/PTX3 or rcHC-HA/PTX3 complex or a composition or mixture containing an nHC-HA/PTX3 or rcHC-HA/PTX3 complex. Biological activity, thus, encompasses therapeutic effects and pharmaceutical activity of nHC-HA/PTX3 or rcHC-HA/PTX3 complexes and compositions and mixtures thereof.

As used herein, the terms “subject”, “individual” and “patient” are used interchangeably. None of the terms are to be interpreted as requiring the supervision of a medical professional (e.g., a doctor, nurse, physician's assistant, orderly, hospice worker). As used herein, the subject is any animal, including mammals (e.g., a human or non-human animal) and non-mammals. In one embodiment of the methods and compositions provided herein, the mammal is a human.

As used herein, the terms “treat,” “treating” or “treatment,” and other grammatical equivalents, include alleviating, abating or ameliorating one or more symptoms of a disease or condition, ameliorating, preventing or reducing the appearance, severity or frequency of one or more additional symptoms of a disease or condition, ameliorating or preventing the underlying metabolic causes of one or more symptoms of a disease or condition, inhibiting the disease or condition, such as, for example, arresting the development of the disease or condition, relieving the disease or condition, causing regression of the disease or condition, relieving a condition caused by the disease or condition, or inhibiting the symptoms of the disease or condition either prophylactically and/or therapeutically. In a non-limiting example, for prophylactic benefit, an rcHC-HA/PTX3 complex, native HC-HA/PTX3 complex, or composition disclosed herein is administered to an individual at risk of developing a particular disorder, predisposed to developing a particular disorder, or to an individual reporting one or more of the physiological symptoms of a disorder.

As used herein, “placenta” refers to the organ that connects a developing fetus to the maternal uterine wall to allow nutrient uptake, waste elimination, and gas exchange via the maternal blood supply. The placenta is composed of three layers. The innermost placental layer surrounding the fetus is called amnion. The allantois is the middle layer of the placenta (derived from the embryonic hindgut); blood vessels originating from the umbilicus traverse this membrane. The outermost layer of the placenta, the chorion, comes into contact with the endometrium. The chorion and allantois fuse to form the chorioallantoic membrane.

As used herein, “chorion” refers to the membrane formed by extraembryonic mesoderm and the two layers of trophoblast. The chorion consists of two layers: an outer formed by the trophoblast, and an inner formed by the somatic mesoderm; the amnion is in contact with the latter. The trophoblast is made up of an internal layer of cubical or prismatic cells, the cytotrophoblast or layer of Langhans, and an external layer of richly nucleated protoplasm devoid of cell boundaries, the syncytiotrophoblast. The avascular amnion is adherent to the inner layer of the chorion.

As used herein, “amnion-chorion” refers to a product comprising amnion and chorion. In some embodiments, the amnion and the chorion are not separated (i.e., the amnion is naturally adherent to the inner layer of the chorion). In some embodiments, the amnion is initially separated from the chorion and later combined with the chorion during processing.

As used herein, “umbilical cord” refers to the organ that connects a developing fetus to the placenta. The umbilical cord is composed of Wharton's jelly, a gelatinous substance made largely from mucopolysaccharides. It contains one vein, which carries oxygenated, nutrient-rich blood to the fetus, and two arteries that carry deoxygenated, nutrient-depleted blood away.

As used herein, “placental amniotic membrane” (PAM) refers to amniotic membrane derived from the placenta. In some embodiments, the PAM is substantially isolated.

As used herein, “umbilical cord amniotic membrane” (UCAM) means amniotic membrane derived from the umbilical cord. UCAM is a translucent membrane. The UCAM has multiple layers: an epithelial layer; a basement membrane; a compact layer; a fibroblast layer; and a spongy layer. It lacks blood vessels or a direct blood supply. In some embodiments, the UCAM comprises Wharton's Jelly. In some embodiments, the UCAM comprises blood vessels and/or arteries. In some embodiments, the UCAM comprises Wharton's Jelly and blood vessels and/or arteries.

As used herein, the terms “purified”, “and “isolated” mean a material (e.g., nHC-HA/PTX3 complex) substantially or essentially free from components that normally accompany it in its native state. In some embodiments, “purified” or “isolated” mean a material (e.g., nHC-HA/PTX3 complex) is about 50% or more free from components that normally accompany it in its native state, for example, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% free from components that normally accompany it in its native state.

### Methods of Treatment

Disclosed herein, in certain embodiments, are methods of treating an individual in need thereof, comprising administering to the individual nHC-HA/PTX3 or rcHC-HA/PTX3 complexes described herein, including methods to directly kill cancer cells, directly inhibit proliferation of cancer cells, reduce the metabolic activity of cancer cells or a combination thereof. In some embodiments, an nHC-HA/PTX3 or rcHC-HA/PTX3 complex is used to directly kill cancer cells. In some embodiments, an nHC-HA/PTX3 or rcHC-HA/PTX3 complex is used to directly inhibit proliferation of cancer cells. In some embodiments, an nHC-HA/PTX3 or rcHC-HA/PTX3 complex is used to reduce the metabolic activity of cancer cells. In some embodiments, cells are from or within a solid tumor.

In some embodiments, the methods of treating an individual in need thereof, comprising administering to the individual nHC-HA/PTX3 or rcHC-HA/PTX3 complexes described herein by any suitable method. In some embodiments, the individual in need thereof has cancer. In some embodiments, the individual in need thereof has an inoperable cancer. In some embodiments, the individual in need thereof has an inoperable cancer selected from the group consisting of pancreatic cancer, prostate cancer, and glioblastoma multiforme. In some embodiments, the individual has a solid tumor. In some embodiments, the individual has a cancer selected from the group consisting of liver cancer, pancreatic cancer, bladder cancer, prostate cancer, lung cancer, non-small cell lung carcinoma, ovarian cancer, breast cancer, melanoma, gastric cancer, colon cancer, colorectal cancer, central nervous system (CNS) cancer, bone cancer, a lymphoma, skin cancer, head and neck cancer, kidney cancer, testicular cancer, uterine cancer, cervical cancer, esophageal cancer, thyroid cancer, salivary gland cancer, adrenal cancer, and gastrointestinal cancer.

In some embodiments, the nHC-HA/PTX3 or rcHC-HA/PTX3 complex is administered to an individual, prior to, during a surgical procedure, or after a surgical procedure. In some embodiments, the surgical procedure comprises excision of a tumor. In some embodiments, the surgical procedure comprises surgical excision, cryoablation, or radiofrequency ablation of a tumor. In some embodiments, the surgical procedure comprises chemotherapy, immunotherapy, or targeted therapy.

In some embodiments, the nHC-HA/PTX3 or rcHC-HA/PTX3 complex is administered up to 1 day, up to 2 days, up to 3 days, up to 5 days, or more than 5 days following excision of a tumor. In some embodiments, the nHC-HA/PTX3 or rcHC-HA/PTX3 complex is administered one week, two weeks, one month, two months, three months, four months, five months, one year, two years, three years, four years, five years, or more than five years following excision of a tumor. In some embodiments, the nHC-HA/PTX3 or rcHC-HA/PTX3 complex is administered singly, or over a time course, such as daily, multiple times weekly, weekly, biweekly, monthly or less frequently following excision of a tumor. In some embodiments, the nHC-HA/PTX3 or rcHC-HA/PTX3 complex is administered singly, or over a time course, such as daily, multiple times weekly, weekly, biweekly, monthly or more frequently following excision of a tumor.

In some embodiments, the nHC-HA/PTX3 or rcHC-HA/PTX3 complex is administered prior to excision of a tumor. In some embodiments, the nHC-HA/PTX3 or rcHC-HA/PTX3 complex is administered up to 1 day, up to 2 days, up to 3 days, up to 5 days, or more than 5 days prior to excision of a tumor. In some embodiments, the nHC-HA/PTX3 or rcHC-HA/PTX3 complex is administered singly, or over a time course, such as daily, multiple times weekly, weekly, biweekly, monthly or less frequently prior to excision of a tumor. In some embodiments, the nHC-HA/PTX3 or rcHC-HA/PTX3 complex is administered singly, or over a time course, such as daily, multiple times weekly, weekly, biweekly, monthly or more frequently prior to excision of a tumor.

In some embodiments, the nHC-HA/PTX3 or rcHC-HA/PTX3 complex is administered to an individual during or after surgical excision, cryoablation, or radiofrequency ablation of a tumor. In some examples, the nHC-HA/PTX3 or rcHC-HA/PTX3 complex is administered to a surgical margin (e.g., an area of apparently tissue around a tumor that has been surgically removed) after surgical excision (e.g., whole or partial), cryoablation, or radiofrequency ablation of a tumor.

In some embodiments, the methods of treating an individual in need thereof, comprising administering to the individual nHC-HA/PTX3 or rcHC-HA/PTX3 complexes by any suitable route of administration. Suitable methods for administration will depend on the disease or condition to be treated. In some embodiments, the nHC-HA/PTX3 or rcHC-HA/PTX3 complexes are administered locally to the site of treatment. In some embodiments, the nHC-HA/PTX3 or rcHC-HA/PTX3 complexes are injected into a tumor, the tissue surrounding the tumor or both. In some embodiments, the nHC-HA/PTX3 or rcHC-HA/PTX3 complexes are injected into a tumor. In some embodiments, the nHC-HA/PTX3 or rcHC-HA/PTX3 complexes are applied to the area surrounding a tumor after the tumor has been surgically removed or treated with cryoablation or radiofrequency ablation. In some embodiments, the nHC-HA/PTX3 or rcHC-HA/PTX3 complexes are administered systemically. Exemplary methods for administration of the nHC-HA/PTX3 or rcHC-HA/PTX3 complexes provided herein include but are not limited to parenteral, enteral, subcutaneous, percutaneous, transdermal, intradermal, intravenous, topical, inhalation, or implantation.

The isolated HC-HA/PTX3 complex is demonstrated herein to directly kill cancer cells. Provided herein, in certain embodiments, are uses of an isolated HC-HA/PTX3 complex, including preparations or compositions comprising HC-HA/PTX3, to kill cancer cells. In some embodiments, provided herein are uses of an isolated HC-HA/PTX3 complex to kill cancer cells in a solid tumor. In some embodiments, provided herein are uses of an isolated HC-HA/PTX3 complex to kill cancer cells in the area surrounding a tumor after the tumor has been surgically removed or treated with cryoablation or radiofrequency ablation. In some embodiments, provided herein are uses of an isolated HC-HA/PTX3 complex to kill cancer cells locally in a subject in need thereof. In some embodiments, provided herein are uses of an isolated HC-HA/PTX3 complex to kill cancer cells systemically in a subject in need thereof.

The isolated HC-HA/PTX3 complex is demonstrated herein to inhibit proliferation of cancer cells. Provided herein, in certain embodiments, are uses of an isolated HC-HA/PTX3 complex, including preparations or compositions comprising HC-HA/PTX3, to inhibit proliferation of cancer cells. In some embodiments, provided herein are uses of an isolated HC-HA/PTX3 complex to inhibit the proliferation of cancer cells in a solid tumor. In some embodiments, provided herein are uses of an isolated HC-HA/PTX3 complex to inhibit the proliferation of cancer cells in the area surrounding a tumor after the tumor has been surgically removed or treated with cryoablation or radiofrequency ablation. In some embodiments, provided herein are uses of an isolated HC-HA/PTX3 complex to inhibit the proliferation of cancer cells locally in a subject in need thereof. In some embodiments, provided herein are uses of an isolated HC-HA/PTX3 complex to inhibit the proliferation of cancer cells systemically in a subject in need thereof. In some embodiments, proliferation is inhibited or decreased by 5-95%, 10-90%, 20-80%, 30-70%, 40-60%, 50-95%, 65-85%, or 75-95%. In some embodiments, proliferation is inhibited or decreased by at least 5%, 10%, 15%, 20%, 25%, 30%, 35%, 40%, 45%, 50%, 55%, 60%, 65%, 70%, 75%, 80%, 85%, 90%, 95%, or greater than 95%. In some embodiments, proliferation is inhibited or decreased by at least 5%. In some embodiments, proliferation is inhibited or decreased by at least 10%. In some embodiments, proliferation is inhibited or decreased by at least 50%.

The isolated HC-HA/PTX3 complex is demonstrated herein to reduce metabolic activity of cancer cells. Provided herein, in certain embodiments, are uses of an isolated HC-HA/PTX3 complex, including preparations or compositions comprising HC-HA/PTX3, to reduce metabolic activity in cancer cells. In some embodiments, provided herein are uses of an isolated HC-HA/PTX3 complex to reduce metabolic activity of cancer cells in a solid tumor. In some embodiments, provided herein are uses of an isolated HC-HA/PTX3 complex to reduce metabolic activity of cancer cells in the area surrounding a tumor after the tumor has been surgically removed or treated with cryoablation or radiofrequency ablation. In some embodiments, provided herein are uses of an isolated HC-HA/PTX3 complex to reduce metabolic activity of cancer cells locally in a subject in need thereof. In some embodiments, provided herein are uses of an isolated HC-HA/PTX3 complex to reduce metabolic activity of cancer cells systemically in a subject in need thereof. In some embodiments, metabolic activity is reduced by 5-95%, 10-90%, 20-80%, 30-70%, 40-60%, 50-95%, 65-85%, or 75-95%. In some embodiments, metabolic activity is reduced by at least 5%, 10%, 15%, 20%, 25%, 30%, 35%, 40%, 45%, 50%, 55%, 60%, 65%, 70%, 75%, 80%, 85%, 90%, 95%, or greater than 95%. In some embodiments, metabolic activity is reduced by at least 5%. In some embodiments, metabolic activity is reduced by at least 10%. In some embodiments, metabolic activity is reduced by at least 50%.

Provided herein, in certain embodiments, are uses of an isolated HC-HA/PTX3 complex, including preparations or compositions comprising HC-HA/PTX3, to increase cancer cell death. In some embodiments, cancer cell death is increased by about 10% to about 25%, about 10% to about 50%, about 20% to about 90%. In some embodiments, cancer cell death is increased by at least 5%, at least 10%, at least 20%, at least 30%, at least 40%, at least 50%, at least 60%, at least 70%, at least 80%, at least 90%, at least 95%, or greater than 95%. In some embodiments, cancer cell death is caused by apoptosis. In some embodiments, cancer cell death is caused by necrosis.

In some embodiments, the cancer cells are from or within a solid tumor. In some embodiments, the solid tumor is a liver cancer, pancreatic cancer, bladder cancer, prostate cancer, lung cancer, non-small cell lung carcinoma, ovarian cancer, breast cancer, melanoma, gastric cancer, colon cancer, colorectal cancer, central nervous system (CNS) cancer, bone cancer, a lymphoma, skin cancer, head and neck cancer, kidney cancer, testicular cancer, uterine cancer, cervical cancer, esophageal cancer, thyroid cancer, salivary gland cancer, adrenal cancer, or a gastrointestinal cancer. In some embodiments, the cancer cells are from a liquid tumor. In some embodiments, the liquid cancer is a lymphoma or leukemia. In some embodiments, the CNS cancer is a glioma or a metastatic cancer. In some embodiments, the glioma is glioblastoma multiforme or anaplastic astrocytoma. In some embodiments, the colon cancer is adenocarcinoma, a carcinoid tumor, a primary colorectal lymphoma, a stromal tumor, or a leiomyosarcoma. In some embodiments, the skin cancer is a melanoma, a basal cell carcinoma, or a squamous cell carcinoma.

### Methods of Producing Isolated nHC-HA/PTX3 Complexes

In some embodiments, isolated native HC-HA/PTX3 (nHC-HA/PTX3) complexes are used in the methods provided herein.

In some embodiments, the isolated nHC-HA/PTX3 complex is isolated from an amniotic tissue. In some embodiments, the isolated nHC-HA/PTX3 complex is isolated from an amniotic membrane or an umbilical cord. In some embodiments, the isolated nHC-HA/PTX3 complex is isolated from fresh, frozen or previously frozen placental amniotic membrane (PAM), fresh, frozen or previously frozen umbilical cord amniotic membrane (UCAM), fresh, frozen or previously frozen placenta, fresh, frozen or previously frozen umbilical cord, fresh, frozen or previously frozen chorion, fresh, frozen or previously frozen amnion-chorion, or any combinations thereof. Such tissues can be obtained from any mammal, such as, for example, but not limited to a human, non-human primate, cow or pig.

In some embodiments, the nHC-HA/PTX3 is purified by any suitable method. In some embodiments, the nHC-HA/PTX3 complex is purified by centrifugation (e.g., ultracentrifugation, gradient centrifugation), chromatography (e.g., ion exchange, affinity, size exclusion, and hydroxyapatite chromatography), tangential flow filtration (TFF), gel filtration, or differential solubility, ethanol precipitation or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference).

In some embodiments, the nHC-HA/PTX3 is isolated from an extract. In some embodiments, the extract is prepared from an amniotic membrane extract. In some embodiments, the extract is prepared from an umbilical cord extract. In some embodiments, the umbilical cord extract comprises umbilical cord stroma and/or Wharton's jelly. In some embodiments, the nHC-HA/PTX3 complex is contained in an extract that is prepared by ultracentrifugation. In some embodiments, the nHC-HA/PTX3 complex is contained in an extract that is prepared by ultracentrifugation using a CsCl/4-6 M guanidine HCl gradient. In some embodiments, the extract is prepared by at least 2 rounds of ultracentrifugation. In some embodiments, the extract is prepared by more than 2 rounds of ultracentrifugation (i.e. nHC-HA/PTX3 2^{nd}). In some embodiments, the extract is prepared by at least 4 rounds of ultracentrifugation (i.e. nHC-HA/PTX3 4^{th}). In some embodiments, the nHC-HA/PTX3 complex comprises a small leucine-rich proteoglycan. In some embodiments, the nHC-HA/PTX3 complex comprises HC1, HA, PTX3 and/or a small leucine-rich proteoglycan.

In some embodiments, ultracentrifugation is performed on an extract prepared by extraction in an isotonic solution. In some embodiments, the isotonic solution is PBS. For example, in some embodiments the tissue is homogenized in PBS to produce a homogenized sample. The homogenized sample is then separated into a soluble portion and insoluble portion by centrifugation. In some embodiments, ultracentrifugation is performed on the soluble portion of the PBS-extracted tissue. In such embodiments, the nHC-HA/PTX3 purified by ultracentrifugation of the PBS-extracted tissue called an nHC-HA/PTX3 soluble complex. In some embodiments, the nHC-HA soluble complex comprises a small leucine-rich proteoglycan. In some embodiments, the nHC-HA/PTX3 soluble complex comprises HC1, HA, PTX3 and/or a small leucine-rich proteoglycan.

In some embodiments, ultracentrifugation is performed on an extract prepared by direct guanidine HCl extraction (e.g. 4-6 M GnHCl) of the amniotic membrane and/or umbilical cord tissue. In some embodiments, the GnHCl extract tissues is then centrifuged to produce GnHCl soluble and GnHCl insoluble portions. In some embodiments, ultracentrifugation is performed on the GnHCl soluble portion. In such embodiments, the nHC-HA/PTX3 purified by ultracentrifugation of the guanidine HCl-extracted tissue is called an nHC-HA/PTX3 insoluble complex. In some embodiments, the nHC-HA insoluble complex comprises a small leucine-rich proteoglycan. In some embodiments, the nHC-HA/PTX3 insoluble complex comprises HC1, HA, PTX3 and/or a small leucine-rich proteoglycan.

In some embodiments, ultracentrifugation is performed on an extract prepared by further guanidine HCl extraction of the insoluble portion of the PBS-extracted tissue. For example, in some embodiments the tissue is homogenized in PBS to produce a homogenized sample. The homogenized sample is then separated into a soluble portion and insoluble portion by centrifugation. The insoluble portion is then further extracted in guanidine HCl (e.g. 4-6 M GnHCl) and centrifuged to produce a guanidine HCl soluble and insoluble portions. In some embodiments, ultracentrifugation is performed on the guanidine HCl soluble portion. In such embodiments, the nHC-HA/PTX3 purified by ultracentrifugation of the guanidine HCl-extracted tissue is called an nHC-HA/PTX3 insoluble complex. In some embodiments, the nHC-HA insoluble complex comprises a small leucine-rich proteoglycan. In some embodiments, the nHC-HA/PTX3 insoluble complex comprises HC1, HA, PTX3 and/or a small leucine-rich proteoglycan.

In some embodiments, the method of purifying the isolated nHC-HA/PTX3 extract comprises: (a) dissolving the isolated extract (e.g. prepared by the soluble or insoluble method described herein) in CsCl/4-6 M guanidine HCl at the initial density of 1.35 g/ml, to generate a CsCl mixture, (b) centrifuging the CsCl mixture at 125,000×g for 48 h at 15° C. to generate a first purified extract and pooling/adjusting HA-containing fractions to the initial density of 1.40 g/ml for the second ultracentrifugation at 125,000×g for 48 h at 15° C., (c) pooling the purified fractions and dialyzing against distilled water to remove CsCl and guanidine HCl, to generate a dialysate. In some embodiments, the method of purifying the isolated extract further comprises (d) mixing the dialysate with 3 volumes of 95% (v/v) ethanol containing 1.3% (w/v) potassium acetate at 0° C. for 1 h, to generate a first dialysate/ethanol mixture, (e) centrifuging the first dialysate/ethanol mixture at 15,000×g, to generate a second purified extract, and (f) extracting the second purified extract. In some embodiments, the method of purifying the isolated extract further comprises: (g) washing the second purified extract with ethanol (e.g., 70% ethanol), to generate a second purified extract/ethanol mixture; (h) centrifuging the second purified extract/ethanol mixture, to generate a third purified extract; and (i) extracting the third purified extract. In some embodiments, the method of purifying the isolated extract further comprises: (j) washing the third purified extract with ethanol (e.g., 70% ethanol), to generate a third purified extract/ethanol mixture; (k) centrifuging the third purified extract/ethanol mixture, to generate a forth purified extract; and (l) extracting the forth purified extract. In some embodiments, the purified extract comprises an nHC-HA/PTX3 complex.

In some embodiments, the nHC-HA/PTX3 complex is purified by immunoaffinity chromatography. In some embodiments, anti HC1 antibodies, anti-HC2 antibodies, or both are generated and affixed to a stationary support. In some embodiments, the unpurified HC-HA complex (i.e., the mobile phase) is passed over the support. In certain instances, the HC-HA complex binds to the antibodies (e.g., via interaction of (a) an anti-HC1 antibody and HC1, (b) an anti-HC2 antibody and HC2, (c) an anti-PTX antibody and PTX3, (d) an anti-SLRP antibody and the SLRP, or (e) any combination thereof). In some embodiments the support is washed (e.g., with PBS) to remove any unbound or loosely bound molecules. In some embodiments, the support is then washed with a solution that enables elution of the nHC-HA/PTX3 complex from the support (e.g., 1% SDS, 6 M guanidine-HCl, or 8 M urea).

In some embodiments, the nHC-HA/PTX3 complex is purified by affinity chromatography. In some embodiments, HABP is generated and affixed to a stationary support. In some embodiments, the unpurified nHC-HA/PTX3 complex (i.e., the mobile phase) is passed over the support. In certain instances, the nHC-HA/PTX3 complex binds to the HABP. In some embodiments the support is washed (e.g., with PBS) to remove any unbound or loosely bound molecules. In some embodiments, the support is then washed with a solution that enables elution of the HC-HA complex from the support.

In some embodiments, the nHC-HA/PTX3 complex is purified by a combination of HABP affinity chromatography, and immunoaffinity chromatography using anti HC1 antibodies, anti-HC2 antibodies, anti-PTX3 antibodies, antibodies against a SLRP or a combination of SLRPs, or any combination of antibodies thereof.

In some embodiments, the nHC-HA/PTX3 complex is purified from the insoluble fraction as described herein using one or more antibodies. In some embodiments, the nHC-HA/PTX3 complex is purified from the insoluble fraction as described herein using anti-SLRP antibodies.

In some embodiments, the nHC-HA/PTX3 complex is purified from the soluble fraction as described herein. In some embodiments, the nHC-HA/PTX3 complex is purified from the soluble fraction as described herein using anti-PTX3 antibodies.

In some embodiments, the nHC-HA/PTX3 complex comprises a small leucine rich proteoglycan (SLRP). In some embodiments, the nHC-HA/PTX3 complex comprises a class I, class II or class III SLRP. In some embodiments, the small leucine-rich proteoglycan is selected from among class I SLRPs, such as decorin and biglycan. In some embodiments, the small leucine-rich proteoglycan is selected from among class II SLRPs, such as fibromodulin, lumican, PRELP (proline arginine rich end leucine-rich protein), keratocan, and osteoadherin. In some embodiments, the small leucine-rich proteoglycan is selected from among class III SLRPs, such as epipycan and osteoglycin. In some embodiments, the small leucine-rich proteoglycan is selected from among bikunin, decorin, biglycan, and osteoadherin. In some embodiments, the small leucine-rich protein comprises a glycosaminoglycan. In some embodiments, the small leucine-rich proteoglycan comprises keratan sulfate.

### Methods of Producing rcHC-HA/PTX3 Complexes

In some embodiments, rcHC-HA/PTX3 complexes are used in the methods provided herein. Such reconstituted HC-HA/PTX3 complexes can be with or without SLRPs.

In some embodiments, a method for generating reconstituted HC-HA/PTX3 complexes comprises (a) contacting hyaluronan (HA) with IαI and TSG-6 to form an HC-HA complex pre-bound to TSG-6 and (b) contacting the HC-HA complex with pentraxin 3 (PTX3) under suitable conditions to form an rcHC-HA/PTX3 complex. Provided herein are rcHC-HA/PTX3 complexes produced by such method. In some embodiments, HC1 of IαI forms a covalent linkage with HA. In some embodiments, the steps (a) and (b) of the method are performed sequentially in order. In some embodiments, the method comprises contacting an HC-HA complex pre-bound to TSG-6 with PTX3. In some embodiments, the hyaluronan (HA) is high molecular weight hyaluronan (HMW HW). In some embodiments, the hyaluronan (HA) is low molecular weight hyaluronan (LMW HW).

In some embodiments, a method for generating reconstituted HC-HA/PTX3 complexes comprises (a) contacting high molecular weight hyaluronan (HMW HA) with IαI and TSG-6 to form an HC-HA complex pre-bound to TSG-6 and (b) contacting the HC-HA complex with pentraxin 3 (PTX3) under suitable conditions to form an rcHC-HA/PTX3 complex. Provided herein are rcHC-HA/PTX3 complexes produced by such method. In some embodiments, HC1 of IαI forms a covalent linkage with HA. In some embodiments, the steps (a) and (b) of the method are performed sequentially in order. In some embodiments, the method comprises contacting an HC-HA complex pre-bound to TSG-6 with PTX3.

In some embodiments, the IαI protein and TSG-6 protein are contacted to the HMW HA at a molar ratio of about 1:1, 2:1, 3:1, 4:1, 5:1, 6:1, 7:1, 8:1, 9:1, 10:1, 15:1, or 20:1 (IαI:TSG-6). In some embodiments the ratio of IαI:TSG-6 ranges from about 1:1 to about 20:1, such as about 1:1 to about 10:1, such as about 1:1 to 5 about:1, such as about 1:1 to about 3:1. In some embodiments, the ratio of IαI:TSG-6 is 3:1 or higher. In some embodiments, the ratio of IαI:TSG-6 is 3:1.

In certain instances, TSG-6 interacts with IαI and forms covalent complexes with HC1 and HC2 of IαI (e.g., HC1⋅TSG-6 and HC2⋅TSG-6). In certain instances, in the presence of HA, the HCs are transferred to HA to form rcHC-HA.

In some embodiments, the step of contacting hyaluronan (HA) with IαI and TSG-6occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments, the step of contacting HA with IαI and TSG-6 occurs for at least 2 hours or longer. In some embodiments, the step of contacting HA with IαI and TSG-6 occurs for at least 2 hours. In some embodiments, the step of contacting HA with IαI and TSG-6 occurs at 37° C. In some embodiments, the step of contacting immobilized HA with IαI and TSG-6 occurs in 5 mM MgCl₂ in PBS. In some embodiments, the hyaluronan (HA) is high molecular weight hyaluronan (HMW HW). In some embodiments, the hyaluronan (HA) is low molecular weight hyaluronan (LMW HW).

In some embodiments, the step of contacting high molecular weight hyaluronan (HMW HA) with IαI and TSG-6 occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments, the step of contacting HMW HA with IαI and TSG-6 occurs for at least 2 hours or longer. In some embodiments, the step of contacting HMW HA with IαI and TSG-6 occurs for at least 2 hours. In some embodiments, the step of contacting HMW HA with IαI and TSG-6 occurs at 37° C. In some embodiments, the step of contacting immobilized HMW HA with IαI and TSG-6 occurs in 5 mM MgCl₂ in PBS.

In some embodiments, the step of contacting PTX3 to an HC-HA complex occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments, the step of contacting PTX3 to an HC-HA complex occurs for at least 2 hours or longer. In some embodiments, the step of contacting PTX3 to an HC-HA complex occurs for at least 2 hours. In some embodiments, the step of contacting PTX3 to an HC-HA complex occurs at 37° C. In some embodiments, the step of contacting PTX3 to an HC-HA complex occurs in 5 mM MgCl₂ in PBS.

In some embodiments, the method comprises contacting hyaluronan (HA) with a pentraxin 3 (PTX3) protein, inter-α-inhibitor (IαI) protein comprising heavy chain 1 (HC1) and heavy chain 2 (HC2), and Tumor necrosis factor α-stimulated gene 6 (TSG-6) simultaneously under suitable conditions to form a HC-HA/PTX3 complex. In some embodiments, the contacting the HA with PTX3, IαI and TSG-6 occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments the step of contacting the HA, PTX3, IαI, and TSG-6 occurs at 37° C. In some embodiments the step of contacting the HA, PTX3, IαI, and TSG-6 occurs in 5 mM MgCl₂ in PBS. In some embodiments, the hyaluronan (HA) is high molecular weight hyaluronan (HMW HW). In some embodiments, the hyaluronan (HA) is low molecular weight hyaluronan (LMW HW).

In some embodiments, the method comprises contacting high molecular weight hyaluronan (HMW HA) with a pentraxin 3 (PTX3) protein, inter-α-inhibitor (IαI) protein comprising heavy chain 1 (HC1) and heavy chain 2 (HC2), and Tumor necrosis factor α-stimulated gene 6 (TSG-6) simultaneously under suitable conditions to form a HC-HA/PTX3 complex. In some embodiments, the contacting the HMW HA with PTX3, IαI and TSG-6 occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments the step of contacting the HMW HA, PTX3, IαI, and TSG-6 occurs at 37° C. In some embodiments the step of contacting the HMW HA, PTX3, IαI, and TSG-6 occurs in 5 mM MgCl₂ in PBS.

In some embodiments, the method comprises contacting hyaluronan (HA) with a pentraxin 3 (PTX3) protein, inter-α-inhibitor (IαI) protein comprising heavy chain 1 (HC1) and heavy chain 2 (HC2), and Tumor necrosis factor α-stimulated gene 6 (TSG-6) sequentially, in any order, under suitable conditions to form a HC-HA/PTX3 complex. In some embodiments, the contacting the HMW HA with PTX3, IαI and TSG-6 occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments the step of contacting the HA, PTX3, IαI, and TSG-6 occurs at 37° C. In some embodiments the step of contacting the HA, PTX3, IαI, and TSG-6 occurs in 5 mM MgCl₂ in PBS. In some embodiments, the hyaluronan (HA) is high molecular weight hyaluronan (HMW HW). In some embodiments, the hyaluronan (HA) is low molecular weight hyaluronan (LMW HW).

In some embodiments, the method comprises contacting high molecular weight hyaluronan (HMW HA) with a pentraxin 3 (PTX3) protein, inter-α-inhibitor (IαI) protein comprising heavy chain 1 (HC1) and heavy chain 2 (HC2), and Tumor necrosis factor α-stimulated gene 6 (TSG-6) sequentially, in any order, under suitable conditions to form a HC-HA/PTX3 complex. In some embodiments, the contacting the HMW HA with PTX3, IαI and TSG-6 occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments the step of contacting the HMW HA, PTX3, IαI, and TSG-6 occurs at 37° C. In some embodiments the step of contacting the HMW HA, PTX3, IαI, and TSG-6 occurs in 5 mM MgCl₂ in PBS.

In some embodiments, the methods for production of an rcHC-HA/PTX3 complex further comprises addition of one or more small leucine rich proteoglycans (SLRPs). In some embodiments, a method for generating reconstituted HC-HA/PTX3 complexes comprises (a) contacting hyaluronan (HA) with IαI and TSG-6 to HA to form an HC-HA complex pre-bound to TSG-6, (b) contacting the HC-HA complex with pentraxin 3 (PTX3) and (c) contacting the HC-HA complex with one or more SLRPS under suitable conditions to form an rcHC-HA/PTX3 complex. In some embodiments, a method for generating reconstituted HC-HA/PTX3 complexes comprises (a) contacting high molecular weight hyaluronan (HMW HA) with IαI and TSG-6 to HA to form an HC-HA complex pre-bound to TSG-6, (b) contacting the HC-HA complex with pentraxin 3 (PTX3) and (c) contacting the HC-HA complex with one or more SLRPS under suitable conditions to form an rcHC-HA/PTX3 complex. Provided herein are rcHC-HA/PTX3 complexes produced by such method. In some embodiments, HC1 of IαI forms a covalent linkage with HA. In some embodiments, the method comprises contacting an HC-HA complex pre-bound to TSG-6 with PTX3. In some embodiments, the steps (a), (b), and (c) of the method are performed sequentially in order. In some embodiments, the steps (a), (b), and (c) of the method are performed simultaneously. In some embodiments, the step (a) of the method is performed and then steps (b) and (c) of the method are performed sequentially in order. In some embodiments, the step (a) of the method is performed and then steps (b) and (c) of the method are performed simultaneously.

In some embodiments, the SLRP is selected from among a class I, class II or class II SLRP. In some embodiments, the SLRP is selected from among class I SLRPs, such as decorin and biglycan. In some embodiments, the small leucine-rich proteoglycan is selected from among class II SLRPs, such as fibromodulin, lumican, PRELP (proline arginine rich end leucine-rich protein), keratocan, and osteoadherin. In some embodiments, the small leucine-rich proteoglycan is selected from among class III SLRPs, such as epipycan and osteoglycin. In some embodiments, the small leucine-rich proteoglycan is selected from among bikunin, decorin, biglycan, and osteoadherin. In some embodiments, the small leucine-rich protein comprises a glycosaminoglycan. In some embodiments, the small leucine-rich proteoglycan comprises keratan sulfate.

### PTX3

In some embodiments, PTX3 for use in the methods is isolated from a cell or a plurality of cells (e.g., a tissue extract). Exemplary cells suitable for the expression of PTX3 include, but are not limited to, animal cells including, but not limited to, mammalian cells, primate cells, human cells, rodent cells, insect cells, bacteria, and yeast, and plant cells, including, but not limited to, algae, angiosperms, gymnosperms, pteridophytes and bryophytes. In some embodiments, PTX3 for use in the methods is isolated from a human cell. In some embodiments, PTX3 for use in the methods is isolated from a cell that is stimulated with one or more proinflammatory cytokines to upregulate PTX3 expression. In some embodiments, the proinflammatory cytokine is IL-1 or TNF-α.

In some embodiments, PTX3 for use in the methods is isolated from an amniotic membrane cell. In some embodiments, PTX3 for use in the methods is isolated from an amniotic membrane cell from an umbilical cord. In some embodiments, the amniotic membrane cell is stimulated with or more proinflammatory cytokines to upregulate PTX3 expression. In some embodiments, the proinflammatory cytokine is IL-1 or TNF-α.

In some embodiments, PTX3 for use in the methods is isolated from an umbilical cord cell. In some embodiments, the umbilical cord cell is stimulated with or more proinflammatory cytokines to upregulate PTX3 expression. In some embodiments, the proinflammatory cytokine is IL-1 or TNF-α.

In some embodiments, PTX3 for use in the methods is isolated from an amniotic epithelial cell. In some embodiments, PTX3 for use in the methods is isolated from an umbilical cord epithelial cell. In some embodiments, the amniotic epithelial cell or umbilical cord epithelial cell is stimulated with or more proinflammatory cytokines to upregulate PTX3 expression. In some embodiments, the proinflammatory cytokine is IL-1 or TNF-α.

In some embodiments, PTX3 for use in the methods is isolated from an amniotic stromal cell. In some embodiments, PTX3 for use in the methods is isolated from an umbilical cord stromal cell. In some embodiments, the amniotic stromal cell or umbilical cord stromal cell is stimulated with or more proinflammatory cytokines to upregulate PTX3 expression. In some embodiments, the proinflammatory cytokine is IL-1 or TNF-α.

In some embodiments, PTX3 for use in the methods is a native PTX3 protein isolated from a cell. In some embodiments, the cell is stimulated with or more proinflammatory cytokines to upregulate PTX3 expression. In some embodiments, the proinflammatory cytokine is IL-1 or TNF-α.

In some embodiments, PTX3 is prepared by recombinant technology. In some embodiments, PTX3 is expressed from a recombinant expression vector. In some embodiments, nucleic acid encoding PTX3 is operably linked to a constitutive promoter. In some embodiments, nucleic acid encoding PTX3 is operably linked to an inducible promoter. In some embodiments, PTX3 is expressed in a transgenic animal. In some embodiments, PTX3 is a recombinant protein. In some embodiments, PTX3 is a recombinant protein isolated from a cell. In some embodiments, PTX3 is a recombinant protein produced in a cell-free extract.

In some embodiments, PTX3 is purified from amniotic membrane, umbilical cord, umbilical cord amniotic membrane, chorionic membrane, amniotic fluid, or a combination thereof. In some embodiments, PTX3 is purified from amniotic membrane cells. In some embodiments, the amniotic membrane cell is an amniotic epithelial cell. In some embodiments, the amniotic membrane cell is an umbilical cord epithelial cell. In some embodiments, the amniotic membrane cell is an amniotic stromal cell. In some embodiments, the amniotic membrane cell is an umbilical cord stromal cell. In some embodiments, the amniotic membrane cell is stimulated with or more proinflammatory cytokines to upregulate PTX3 expression. In some embodiments, the proinflammatory cytokine is IL-1 or TNF-α.

In some embodiments, PTX3 is not isolated from a cell or a plurality of cells (e.g., a tissue extract).

In some embodiments, PTX3 comprises a fragment of PTX3 sufficient to facilitate the formation of rcHC-HA/PTX3 complex. Variants of PTX3 for use in the provided methods include variants with an amino acid modification that is an amino acid replacement (substitution), deletion or insertion. In some embodiments, such modification improves one or more properties of the PTX3 polypeptides such as improving the one or more therapeutic properties of the rcHC-HA/PTX3 complex (e.g., anti-inflammatory, anti-immune, anti-angiogenic, anti-scarring, anti-adhesion, regeneration or other therapeutic activities as described herein).

In some embodiments PTX3 protein is obtained from a commercial source. An exemplary commercial source for PTX3 is, but is not limited to, PTX3 (Catalog No. 1826-TS; R&D Systems, Minneapolis, Minn.).

In some embodiments, the PTX3 protein used in the methods is a multimeric protein. In some embodiments, the PTX3 protein used in the methods is a homomultimer. In some embodiments, the homomultimer is a dimer, trimer, tetramer, hexamer, pentamer, or octamer. In some embodiments, the PTX3 homomultimer is a trimer, tetramer, or octamer. In particular embodiments, the PTX3 homomultimer is an octamer. In some embodiments, the multimerization domain is modified to improve multimerization of the PTX3 protein. In some embodiments, the multimerization domain is replaced with a heterogeneous multimerization domain (e.g., an Fc multimerization domain or leucine zipper) that when fused to PTX3 improves the multimerization of PTX3.

### TSG-6

In some embodiments, TSG-6 for use in the methods is isolated from a cell or a plurality of cells (e.g., a tissue extract). Exemplary cells suitable for the expression of TSG-6 include, but are not limited to, animal cells including, but not limited to, mammalian cells, primate cells, human cells, rodent cells, insect cells, bacteria, and yeast, and plant cells, including, but not limited to, algae, angiosperms, gymnosperms, pteridophytes and bryophytes. In some embodiments, TSG-6 for use in the methods is isolated from a human cell. In some embodiments, TSG-6 for use in the methods is isolated from a cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an amniotic membrane cell. In some embodiments, TSG-6 for use in the methods is isolated from an amniotic membrane cell from an umbilical cord. In some embodiments, TSG-6 for use in the methods is isolated from an amniotic membrane cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an umbilical cord cell. In some embodiments, TSG-6 for use in the methods is isolated from an umbilical cord cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an amniotic epithelial cell. In some embodiments, TSG-6 for use in the methods is isolated from an umbilical cord epithelial cell. In some embodiments, TSG-6 for use in the methods is isolated from an amniotic epithelial cell or an umbilical cord epithelial cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an amniotic stromal cell. In some embodiments TSG-6 for use in the methods is isolated from an umbilical cord stromal cell. In some embodiments, TSG-6 for use in the methods is isolated from an amniotic stromal cell or an umbilical cord stromal cell that is stimulated with one or more proinflammatory cytokines to upregulate TSG-6 expression. In some embodiments, the proinflammatory cytokine is IL-1 or TNF-α.

In some embodiments, TSG-6 for use in the methods is a native TSG-6 protein isolated from a cell. In some embodiments, the cell is stimulated with or more proinflammatory cytokines to upregulate TSG-6 expression. In some embodiments, the proinflammatory cytokine is IL-1 or TNF-α.

In some embodiments, TSG-6 is prepared by recombinant technology. In some embodiments, TSG-6 is expressed from a recombinant expression vector. In some embodiments, nucleic acid encoding TSG-6 is operably linked to a constitutive promoter. In some embodiments, nucleic acid encoding TSG-6 is operably linked to an inducible promoter. In some embodiments, TSG-6 is expressed in a transgenic animal. In some embodiments, TSG-6 is a recombinant protein. In some embodiments, TSG-6 is a recombinant protein isolated from a cell. In some embodiments, TSG-6 is a recombinant protein produced in a cell-free extract.

In some embodiments, TSG-6 is purified from amniotic membrane, amniotic membrane, chorionic membrane, amniotic fluid, or a combination thereof. In some embodiments, TSG-6 is purified from amniotic membrane cells. In some embodiments, the amniotic membrane cell is an amniotic epithelial cell. In some embodiments, the amniotic epithelial cell is an umbilical cord epithelial cell. In some embodiments, the amniotic membrane cell is an amniotic stromal cell. In some embodiments, the amniotic membrane cell is an umbilical cord stromal cell. In some embodiments, the amniotic membrane cell is stimulated with or more proinflammatory cytokines to upregulate TSG-6 expression. In some embodiments, the proinflammatory cytokine is IL-1 or TNF-α.

In some embodiments, TSG-6 is not isolated from a cell or a plurality of cells (e.g., a tissue extract).

In some embodiments, TSG-6 comprises a fragment of TSG-6 that is sufficient to facilitate or catalyze the transfer HC1 of IαI to HA. In some embodiments, TSG-6 comprises the link module of TSG-6.

In some embodiments, TSG-6 comprises an affinity tag. Exemplary affinity tags include but are not limited to a hemagglutinin tag, a poly-histidine tag, a myc tag, a FLAG tag, a glutathione-S-transferase (GST) tag. Such affinity tags are well known in the art for use in purification. In some embodiments, such an affinity tag incorporated into the TSG-6 polypeptide as a fusion protein or via a chemical linker. In some embodiments, TSG-6 comprises an affinity tag and the unbound TSG-6 is removed from the rcHC-HA/PTX3 complex by affinity purification.

In some embodiments TSG-6 protein is obtained from a commercial source. An exemplary commercial source for TSG-6 is, but is not limited to, TSG-6 (Catalog No. 2104-TS R&D Systems, Minneapolis, Minn.).

### IαI

In some embodiments, the IαI comprises an HC1 chain. In some embodiments, the IαI comprises an HC1 and an HC2 chain. In some embodiments, the IαI comprises an HC1, and HC2 chain and bikunin. In some embodiments, the IαI comprises an HC1, and HC2 chain and bikunin linked by a chondroitin sulfate chain.

In some embodiments, IαI is isolated from a biological sample. In some embodiments the biological sample is a biological sample from a mammal. In some embodiments, the mammal is a human. In some embodiments, the biological sample is a blood, serum, plasma, liver, amniotic membrane, chorionic membrane or amniotic fluid sample. In some embodiments, the biological sample is a blood, serum, or plasma sample. In some embodiments, the biological sample is a blood sample. In some embodiments, the biological sample is a serum sample. In some embodiments, the biological sample is a plasma sample. In some embodiments, the IαI is purified from human blood, plasma or serum. In some embodiments, IαI is isolated from human serum. In some embodiments, IαI is not isolated from serum. In some embodiments, IαI for use in the methods is produced in an amniotic membrane cell. In some embodiments, IαI for use in the methods is produced in an umbilical cord cell. In some embodiments, IαI for use in the methods is produced in an amniotic membrane cell from an umbilical cord. In some embodiments, IαI for use in the methods is produced in an amniotic epithelial cell. In some embodiments, IαI for use in the methods is produced in an umbilical cord epithelial cell. In some embodiments, IαI for use in the methods is produced in an amniotic stromal cell. In some embodiments, IαI for use in the methods is produced in an umbilical cord stromal cell. In some embodiments, IαI for use in the methods is produced in a hepatic cell. In some embodiments, IαI is prepared by recombinant technology.

In some embodiments, HC1 of IαI is isolated from a biological sample. In some embodiments the biological sample is a biological sample from a mammal. In some embodiments, the mammal is a human. In some embodiments, the biological sample is a blood, serum, plasma, liver, amniotic membrane, chorionic membrane or amniotic fluid sample. In some embodiments, the biological sample is a blood, serum, or plasma sample. In some embodiments, the biological sample is a blood sample. In some embodiments, the biological sample is a serum sample. In some embodiments, the biological sample is a plasma sample. In some embodiments, the HC1 of IαI is purified from human blood, plasma or serum. In some embodiments, IαI is isolated from human serum. In some embodiments, HC1 of IαI is not purified from serum. In some embodiments, HC1 of IαI is prepared by recombinant technology. In some embodiments, HC1 of IαI is purified from hepatic cells. In some embodiments, HC1 of IαI is purified from amniotic membrane cells. In some embodiments, HC1 of IαI is purified from amniotic epithelial cells or umbilical cord epithelial cells. In some embodiments, HC1 of IαI is purified from amniotic stromal cells or umbilical cord stromal cells.

In some embodiments, HC2 of IαI is isolated from a biological sample. In some embodiments the biological sample is a biological sample from a mammal. In some embodiments, the mammal is a human. In some embodiments, the biological sample is a blood, serum, plasma, liver, amniotic membrane, chorionic membrane or amniotic fluid sample. In some embodiments, the biological sample is a blood, serum, or plasma sample. In some embodiments, the biological sample is a blood sample. In some embodiments, the biological sample is a serum sample. In some embodiments, the biological sample is a plasma sample. In some embodiments, the HC2 of IαI is purified from human blood, plasma or serum. In some embodiments, HC2 of IαI is isolated from human serum. In some embodiments, HC2 of IαI is isolated from human serum. In some embodiments, HC2 of IαI is not isolated from blood serum. In some embodiments, HC2 of IαI is prepared by recombinant technology. In some embodiments, HC2 of IαI is purified from hepatic cells. In some embodiments, HC2 of IαI is purified from amniotic membrane cells. In some embodiments, HC2 of IαI is purified from amniotic epithelial cells or umbilical cord epithelial cells. In some embodiments, HC2 of IαI is purified from amniotic stromal cells or umbilical cord stromal cells.

### HA

In some embodiments, HA is purified from a cell, tissue or a fluid sample. In some embodiments, HA is obtained from a commercial supplier (e.g., Sigma Aldrich or Advanced Medical Optics, Irvine, Calif. (e.g., Healon)). In some embodiments, HA is obtained from a commercial supplier as a powder. In some embodiments, HA is expressed in a cell. Exemplary cells suitable for the expression of HA include, but are not limited to, animal cells including, but not limited to, mammalian cells, primate cells, human cells, rodent cells, insect cells, bacteria, and yeast, and plant cells, including, but not limited to, algae, angiosperms, gymnosperms, pteridophytes and bryophytes. In some embodiments, HA is expressed in a human cell. In some embodiments, HA is expressed in a transgenic animal. In some embodiments, HA is obtained from a cell that expresses a hyaluronan synthase (e.g., HAS1, HAS2, and HAS3). In some embodiments, the cell contains a recombinant expression vector that expresses an HA synthase. In certain instances, an HA synthase lengthens hyaluronan by repeatedly adding glucuronic acid and N-acetylglucosamine to the nascent polysaccharide as it is extruded through the cell membrane into the extracellular space.

HA for use in the methods, in some embodiments, is high molecular weight (HMW) HA. In some embodiments, the weight average molecular weight of HMW HA is greater than about 500 kilodaltons (kDa), such as, for example, between about 500 kDa and about 10,000 kDa, between about 800 kDa and about 8,500 kDa, between about 1100 kDa and about 5,000 kDa, or between about 1400 kDa and about 3,500 kDa. In some embodiments, the weight average molecular weight of HMW HA is about 3000 kDa.

Additional Components

In some embodiments, one or more additional components are added to generate an rcHC-HA/PTX3 complex. In some embodiments, a small leucine rich proteoglycan (SLRP) is added to generate an rcHC-HA/PTX3 complex. In some embodiments, the SLRP is a class I, class II or class II SLRP. In some embodiments, the SLRP is selected from among class I SLRPs, such as decorin and biglycan. In some embodiments, the SLRP is selected from among class II SLRPs, such as fibromodulin, lumican, PRELP (proline arginine rich end leucine-rich protein), keratocan, and osteoadherin. In some embodiments, the SLRP is selected from among class III SLRPs, such as epipycan and osteoglycin. In some embodiments, the SLRP is selected from among bikunin, decorin, biglycan, and osteoadherin. In some embodiments, the SLRP comprises a glycosaminoglycan. In some embodiments, the SLRP comprises keratan sulfate.

In some embodiments, HMW HA is immobilized by any suitable method. In some embodiments, HMW HA is immobilized to a solid support, such as culture dish, bead, a column or other suitable surfaces, such as, for example, a surface of an implantable medical device or a portion thereof or on a surface that is subsequently connected to or combined with an implantable medical device as described herein. In some embodiments, HMW HA is immobilized directly to the solid support, such as by chemical linkage. In some embodiments, HMW HA is attached indirectly to the solid support via a linker or an intermediary protein. Numerous heterobifunctional cross-linking reagents that are used to form covalent bonds between amino groups and thiol groups and to introduce thiol groups into proteins, are known to those of skill in this art. In some embodiments, HMW HA is immobilized directly to the solid support via crosslinking to the solid support. In some embodiments, HMW HA is immobilized directly to the solid support without crosslinking to the solid support. In some embodiments, HMW HA is immobilized directly to the solid support as a coating. In some embodiments, HMW HA is immobilized to a Covalink™-NH surface. In some embodiments, HMW HA is immobilized directly to the solid support as a coating. In some embodiments, HMW HA is immobilized to a Covalink™-NH surface for about 16 h at 4° C.

In some embodiments, the method comprises immobilizing HMW HA to a solid surface via direct linkage to a solid support (i.e. without an intermediary protein). In some embodiments, the solid support is washed to remove unbound HMW HA prior to contacting the immobilized HA with IαI, TSG-6, and PTX3. In some embodiments, the solid support is washed with washes of 8 M GnHCl and PBS to remove unbound HMW HA prior to contacting the immobilized HA with IαI, TSG-6, and PTX3.

In some embodiments, the method comprises immobilizing HA to a solid surface via an intermediary protein or a linker. In some embodiments, the linker is a peptide linker. In some embodiments, the intermediary protein is an HA binding protein (HABP). In some embodiments, HABP is first attached to a solid support (e.g., by cross-linking, chemical linkage or via a chemical linker). In some embodiments, the solid support comprising HABP is then contacted with HA (e.g., HMW HA) to immobilize HA to the solid support via binding of the HABP to HA. In some embodiments, the solid support is washed to remove unbound HMW HA prior to contacting the immobilized HMW HA with IαI, TSG-6, and PTX3. In some embodiments, the solid support is washed with washes of 8 M GnHCl and PBS to remove unbound HMW HA prior to contacting the immobilized HA with IαI, TSG-6, and PTX3.

In some embodiments, the method comprises immobilizing HA to a solid surface via attachment of a peptide linker to the solid support and attachment HA to the peptide linker. In some embodiments, the peptide linker comprises a protease cleavage site.

In some embodiments, the method comprises immobilizing HA to a solid surface via attachment of a cleavable chemical linker, such as, but not limited to a disulfide chemical linker.

In some embodiments, the HABP selected for use in the methods is an HABP that is dissociated from HA following formation of the rcHC-HA/PTX3 complex. In some embodiments, the HABP non-covalently binds to HA. In some embodiments, the method further comprises dissociating the rcHC-HA/PTX3 complex from HABP using one or more dissociating agents. Dissociating agents for the disruption of non-covalent interactions (e.g., guanidine hydrochloride, urea and various detergents, e.g., SDS) are known in the art. In some embodiments the dissociating agent is urea. In some embodiments the dissociating agent is guanidine hydrochloride. In some embodiments, the dissociation agent is about 4M to about 8M guanidine-HCl. In some embodiments, the dissociation agent is about 4M, about 5M, about 6M, about 7M, about 8M guanidine-HCl. In some embodiments, the dissociation agent is about 4M to about 8M guanidine-HCl in PBS at pH 7.5.

In some embodiments, such dissociating agents are employed to dissociate the rcHC-HA/PTX3 complex from an intermediary HABP. An HABP for use in the methods typically is selected such that the binding affinity for HA is strong enough to permit assembly of the rcHC-HA/PTX3 complex but is dissociated from the rcHC-HA/PTX3 complex with a suitable dissociation agent. In some embodiments the dissociating agent is guanidine hydrochloride.

Exemplary HABPs for use with the methods provided herein include, but are not limited to, HAPLN1, HAPLN2, HAPLN3, HAPLN4, aggrecan, versican, neurocan, brevican, phosphacan, TSG-6, CD44, stabilin-1, stabilin-2, or portions thereof (e.g., link modules thereof) sufficient to bind HA. In some embodiments, the HABP is versican. In some embodiments, the HABP is a recombinant protein. In some embodiments, the HABP is a recombinant mammalian protein. In some embodiments, the HABP is a recombinant human protein. In some embodiments, the HABP is a recombinant versican protein or a portion thereof sufficient to bind to HA. In some embodiments, the HABP is a recombinant aggrecan protein or a portion thereof sufficient to bind to HA. In some embodiments, the HABP is a native HABP or a portion thereof sufficient to bind to HA. In some embodiments, the native HABP is isolated from mammalian tissue or cells. In some embodiments, the HABP is isolated from bovine nasal cartilage (e.g. HABP from Seikagaku which contains the HA binding domains of aggrecan and link protein).

In some embodiments, the HABP comprises a link module of HAPLN1, HAPLN2, HAPLN3, HAPLN4, aggrecan, versican, neurocan, brevican, phosphacan, TSG-6, CD44, stabilin-1, or stabilin-2. In some embodiments, the HABP comprises a link module of versican. In some embodiments, the HABP comprising a link module is a recombinant protein. In some embodiments, the HABP comprising a link module of versican is a recombinant protein.

In some embodiments, an intermediary protein, such as an HABP, contains a proteolytic cleavage sequence that is recognized by and is hydrolyzed by a site-specific protease, such as furin, 3C protease, caspase, matrix metalloproteinase or TEV protease. In such embodiments, assembled rcHC-HA/PTX3 complexes are released from the solid support by contacting the immobilized complexes with a protease that cleaves the specific cleavage sequence.

In some embodiments, the rcHC-HA/PTX3 complex is purified. In some embodiments, the rcHC-HA/PTX3 complex is purified by any suitable method or combination of methods. The embodiments described below are not intended to be exclusive, only exemplary.

In some embodiments, the rcHC-HA/PTX3 complex is purified by chromatography (e.g., ion exchange, affinity, size exclusion, and hydroxyapatite chromatography), tangential flow filtration (TFF), gel filtration, centrifugation (e.g., gradient centrifugation), or differential solubility, ethanol precipitation or by any other available technique for the purification of proteins.

In some embodiments, the rcHC-HA/PTX3 complex is purified by immunoaffinity chromatography. In some embodiments antibodies are generated against a component of the rcHC-HA/PTX3 complex (e.g., anti-HC1, anti-PTX3, an antibody against one or more SLRPs of the rcHC-HA/PTX3 complex, e.g., anti-bikunin, anti-decorin, anti-biglycan, or anti-osteoadherin) and affixed to a solid support. In some embodiments, the unpurified rcHC-HA/PTX3 complex (i.e., the mobile phase) is passed over the support. In certain instances, the rcHC-HA/PTX3 complex binds to the antibodies. In some embodiments, the support is washed (e.g., with PBS) to remove any unbound or loosely bound molecules. In some embodiments, the support is then washed with a solution that enables elution of the rcHC-HA/PTX3 complex from the support (e.g., 1% SDS, 6M guanidine-HCl, or 8M urea). In some embodiments, the dissociating agent is removed from the dissociated rcHC-HA/PTX3 complex. In some embodiments, the dissociating agent is removed from the dissociated rcHC-HA/PTX3 complex by a method including, but not limited to, ion-exchange chromatography, dialysis, tangential flow filtration (TFF), gel filtration chromatography, ultrafiltration, or diafiltration.

In some embodiments, the rcHC-HA/PTX3 complex is purified by affinity chromatography. In some embodiments, an HABP is employed to bind to the rcHC-HA/PTX3 complex for purification of the complex and affixed to a stationary support. In some embodiments, the unpurified rcHC-HA/PTX3 complex (i.e., the mobile phase) is passed over the support. In certain instances, the rcHC-HA/PTX3 complex binds to the HABP. In some embodiments the support is washed (e.g., with PBS) to remove any unbound or loosely bound molecules. In some embodiments, the support is then washed with a solution (e.g., a dissociating agent) that enables elution of the rcHC-HA/PTX3 complex from the support. In some embodiments, the dissociating agent is removed from the dissociated rcHC-HA/PTX3 complex by a method including, but not limited to, ion-exchange chromatography, dialysis, tangential flow filtration (TFF), gel filtration chromatography, ultrafiltration, or diafiltration.

In some embodiments, the rcHC-HA/PTX3 complex is purified by a combination of HABP affinity chromatography, and immunoaffinity chromatography using antibodies against one or more components of the rcHC-HA/PTX3 complex.

In some embodiments, one or more components of the rcHC-HA/PTX3 complex disclosed herein comprise an affinity tag (e.g., a fusion protein of PTX3 or HC1 with an affinity tag). Exemplary affinity tags that are incorporated into one or more components of the rcHC-HA/PTX3 complex in some embodiments include, but are not limited to, a hemagglutinin tag, poly-histidine, a myc tag, a FLAG tag, or glutathione-S-transferase sequence. In some embodiments, the ligand for the affinity tag is affixed to the solid support. In some embodiments, the unpurified rcHC-HA/PTX3 complex is passed over the support. In certain instances, the rcHC-HA/PTX3 complex binds to the ligand. In some embodiments the support is washed (e.g., with PBS) to remove any unbound or loosely bound molecules. In some embodiments, the support is then washed with a solution that enables elution of an rcHC-HA/PTX3 complex disclosed herein from the support. In some embodiments, the elution agent is removed from the dissociated rcHC-HA/PTX3 complex by a method including, but not limited to, ion-exchange chromatography, dialysis, tangential flow filtration (TFF), gel filtration chromatography, ultrafiltration, or diafiltration.

In some embodiments, the PTX3, TSG-6, and/or HC1 are conjugated to a label. A “label” refers to a detectable compound or composition which is conjugated directly or indirectly to a polypeptide so as to generate a labeled polypeptide. In some embodiments, the label is detectable by itself (e.g., radioisotope labels or fluorescent labels) or, in the case of an enzymatic label, catalyzes chemical alteration of a substrate compound composition which is detectable. Non-limiting examples of labels include fluorogenic moieties, dyes, fluorescent tags, green fluorescent protein, or luciferase.

### Methods of Assessing the Activity of nHC-HA/PTX3 and rcHC-HA/PTX3 Complexes

The properties of nHC-HA/PTX3 and rcHC-HA/PTX3 complexes provided herein are assessed by any suitable method including, in vitro and in vivo methods. Exemplary in vitro methods are provided herein and include, but are not limited, to cell culture methods that assess the ability of nHC-HA/PTX3 or rcHC-HA/PTX3 complexes to promote attachment of macrophages to the immobilized nHC-HA/PTX3 or rcHC-HA/PTX3 complexes, to inhibit or reduce aggregation of macrophages, to promote apoptosis of neutrophils, macrophage phagocytosis of apoptotic neutrophils, and M2 polarization of stimulated macrophages. In some embodiments, the macrophages used in the assay are stimulated, such as by exposure to LPS or IFN-γ. In some embodiments, the gene or protein expression in stimulated macrophages is assessed following contact with nHC-HA/PTX3 or rcHC-HA/PTX3 complexes. In such methods of assessing activity of nHC-HA/PTX3 or rcHC-HA/PTX3 complex, a suitable control is employed for comparison. In some embodiments, the control is the absence of treatment with an nHC-HA/PTX3 or rcHC-HA/PTX3 complex (i.e. a negative control).

In some embodiments, the activity of an rcHC-HA/PTX3 complex is compared to the activity of a native HC-HA/PTX3 complex. In some embodiments the native HC-HA/PTX3 is isolated from amniotic membrane.

In some embodiments, gene expression in treated macrophages is assessed by PCR, RT-PCR, Northern blotting, western blotting, dot blotting, immunohistochemistry, chromatography or other suitable method of detecting proteins or nucleic acids. In some embodiments, the level of expression of IL-10, IL-12, IL23, LIGHT and SPHK1 is assessed.

### Pharmaceutical Compositions

Disclosed herein, in certain embodiments, are pharmaceutical compositions comprising nHC-HA/PTX3 or rcHC-HA/PTX3 complexes described herein. Disclosed herein, in certain embodiments, are pharmaceutical compositions comprising nHC-HA/PTX3 or rcHC-HA/PTX3 complexes produced by the methods provided herein. In some embodiments, the pharmaceutical compositions are formulated in a conventional manner using one or more physiologically acceptable carriers including excipients and auxiliaries which facilitate processing of an nHC-HA/PTX3 or rcHC-HA/PTX3 complex into preparations which are suitable for pharmaceutical use. Proper formulation is dependent upon the route of administration selected. Any of the well-known techniques, carriers, and excipients can be used as suitable and as understood in the art.

Disclosed herein, in certain embodiments, is a pharmaceutical composition comprising an nHC-HA/PTX3 or rcHC-HA/PTX3 complex disclosed herein. In some embodiments, the pharmaceutical composition further comprises at least one pharmaceutically acceptable carrier. In some embodiments, the pharmaceutical composition further comprises an adjuvant, excipient, preservative, agent for delaying absorption, filler, binder, adsorbent, buffer, and/or solubilizing agent. Exemplary pharmaceutical compositions that are formulated to contain an nHC-HA/PTX3 or rcHC-HA/PTX3 complex provided herein include, but are not limited to, a solution, suspension, emulsion, syrup, granule, powder, ointment, tablet, capsule, pill, tincture, transdermal system, ointment, lotion, cream, paste, foam, gel, or an aerosol.

Dosage Forms

In some embodiments, an nHC-HA/PTX3 or rcHC-HA/PTX3 complex disclosed herein is administered as an aqueous suspension. In some embodiments, an aqueous suspension comprises water, Ringer's solution and/or isotonic sodium chloride solution. In some embodiments, an aqueous suspension comprises a sweetening or flavoring agent, coloring matters or dyes and, if desired, emulsifying agents or suspending agents, together with diluents water, ethanol, propylene glycol, glycerin, or combinations thereof. In some embodiments, an aqueous suspension comprises a suspending agent. In some embodiments, an aqueous suspension comprises sodium carboxymethylcellulose, methylcellulose, hydroxypropylmethyl-cellulose, sodium alginate, polyvinyl-pyrrolidone, gum tragacanth and/or gum acacia. In some embodiments, an aqueous suspension comprises a dispersing or wetting agent. In some embodiments, an aqueous suspension comprises a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. In some embodiments, an aqueous suspension comprises a preservative. In some embodiments, an aqueous suspension comprises ethyl, or n-propyl p-hydroxybenzoate. In some embodiments, an aqueous suspension comprises a sweetening agent. In some embodiments, an aqueous suspension comprises sucrose, saccharin or aspartame.

In some embodiments, an nHC-HA/PTX3 or rcHC-HA/PTX3 complex disclosed herein is administered as an oily suspension. In some embodiments, an oily suspension is formulated by suspending the active ingredient in a vegetable oil (e.g., arachis oil, olive oil, sesame oil or coconut oil), or in mineral oil (e.g., liquid paraffin). In some embodiments, an oily suspension comprises a thickening agent (e.g., beeswax, hard paraffin or cetyl alcohol). In some embodiments, an oily suspension comprises sweetening agents (e.g., those set forth above). In some embodiments, an oily suspension comprises an anti-oxidant (e.g., butylated hydroxyanisol or alpha-tocopherol).

In some embodiments, an nHC-HA/PTX3 or rcHC-HA/PTX3 complex disclosed herein is formulated for parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural and/or subcutaneous). In some embodiments, an nHC-HA/PTX3 or rcHC-HA/PTX3 complex disclosed herein is administered as a sterile solution, suspension or emulsion.

In some embodiments, a formulation for parenteral administration includes aqueous and/or non-aqueous (oily) sterile injection solutions of an nHC-HA/PTX3 or rcHC-HA/PTX3 complex disclosed herein, which in some embodiments, contain antioxidants, buffers, bacteriostats and/or solutes which render the formulation isotonic with the blood of the intended recipient; and/or aqueous and/or non-aqueous sterile suspensions which in some embodiments, include a suspending agent and/or a thickening agent. In some embodiments, a formulation for parenteral administration includes suitable stabilizers or agents which increase the solubility of an nHC-HA/PTX3 or rcHC-HA/PTX3 complex disclosed herein to allow for the preparation of highly concentrated solutions.

In some embodiments, an nHC-HA/PTX3 or rcHC-HA/PTX3 complex disclosed herein is administered as an oil-in-water micro-emulsion where the active ingredient is dissolved in the oily phase. In some embodiments, an nHC-HA/PTX3 or rcHC-HA/PTX3 complex disclosed herein is dissolved in a fatty oil (e.g., sesame oil, or synthetic fatty acid esters, (e.g., ethyl oleate or triglycerides, or liposomes. In some embodiments, an nHC-HA/PTX3 or rcHC-HA/PTX3 complex disclosed herein is dissolved in a mixture of soybean oil and/or lecithin. In some embodiments, the oil solution is introduced into a water and glycerol mixture and processed to form a micro-emulsion.

In some embodiments, a composition formulated for parenteral administration is administered as a single bolus shot. In some embodiments, a composition formulated for parenteral administration is administered via a continuous intravenous delivery device (e.g., Deltec CADD-PLUS™ model 5400 intravenous pump).

In some embodiments, a formulation for injection is presented in unit dosage form, e.g., in ampoules or in multi-dose containers, with an added preservative. In some embodiments, a formulation for injection is stored in powder form or in a freeze-dried (lyophilized) condition requiring only the addition of the sterile liquid carrier, for example, saline or sterile pyrogen-free water, immediately prior to use.

In some embodiments, an nHC-HA/PTX3 or rcHC-HA/PTX3 complex disclosed herein is formulated for topical administration. Topical formulations include, but are not limited to, ointments, creams, lotions, solutions, pastes, gels, films, sticks, liposomes, nanoparticles. In some embodiments, a topical formulation is administered by use of a patch, bandage or wound dressing.

In some embodiments, an nHC-HA/PTX3 or rcHC-HA/PTX3 complex disclosed herein is formulated as composition is in the form of a solid, a cross-linked gel, or a liposome. In some embodiments, an nHC-HA/PTX3 or rcHC-HA/PTX3 complex disclosed herein is formulated as an insoluble cross-linked hydrogel.

In some embodiments, a topical formulation comprises a gelling (or thickening) agent. Suitable gelling agents include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, acacia (gum arabic), agar, aluminum magnesium silicate, sodium alginate, sodium stearate, bladderwrack, bentonite, carbomer, carrageenan, carbopol, xanthan, cellulose, microcrystalline cellulose (MCC), ceratonia, chondrus, dextrose, furcellaran, gelatin, ghatti gum, guar gum, hectorite, lactose, sucrose, maltodextrin, mannitol, sorbitol, honey, maize starch, wheat starch, rice starch, potato starch, gelatin, sterculia gum, polyethylene glycol (e.g. PEG 200-4500), gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), or combinations thereof.

In some embodiments, a topical formulation disclosed herein comprises an emollient. Emollients include, but are not limited to, castor oil esters, cocoa butter esters, safflower oil esters, cottonseed oil esters, corn oil esters, olive oil esters, cod liver oil esters, almond oil esters, avocado oil esters, palm oil esters, sesame oil esters, squalene esters, kukui oil esters, soybean oil esters, acetylated monoglycerides, ethoxylated glyceryl monostearate, hexyl laurate, isohexyl laurate, isohexyl palmitate, isopropyl palmitate, methyl palmitate, decyloleate, isodecyl oleate, hexadecyl stearate decyl stearate, isopropyl isostearate, methyl isostearate, diisopropyl adipate, diisohexyl adipate, dihexyldecyl adipate, diisopropyl sebacate, lauryl lactate, myristyl lactate, and cetyl lactate, oleyl myristate, oleyl stearate, and oleyl oleate, pelargonic acid, lauric acid, myristic acid, palmitic acid, stearic acid, isostearic acid, hydroxystearic acid, oleic acid, linoleic acid, ricinoleic acid, arachidic acid, behenic acid, erucic acid, lauryl alcohol, myristyl alcohol, cetyl alcohol, hexadecyl alcohol, stearyl alcohol, isostearyl alcohol, hydroxystearyl alcohol, oleyl alcohol, ricinoleyl alcohol, behenyl alcohol, erucyl alcohol, 2-octyl dodecanyl alcohol, lanolin and lanolin derivatives, beeswax, spermaceti, myristyl myristate, stearyl stearate, carnauba wax, candelilla wax, lecithin, and cholesterol.

In some embodiments, an nHC-HA/PTX3 or rcHC-HA/PTX3 complex disclosed herein is formulated with one or more natural polymers. In some embodiments, an nHC-HA/PTX3 or rcHC-HA/PTX3 complex disclosed herein is formulated with a natural polymer that is fibronectin, collagen, laminin, keratin, fibrin, fibrinogen, hyaluronic acid, heparan sulfate, chondroitin sulfate. In some embodiments, an nHC-HA/PTX3 or rcHC-HA/PTX3 complex disclosed herein is formulated with a polymer gel formulated from a natural polymer. In some embodiments, an nHC-HA/PTX3 or rcHC-HA/PTX3 complex disclosed herein is formulated with a polymer gel formulated from a natural polymer, such as, but not limited to, fibronectin, collagen, laminin, keratin, fibrin, fibrinogen, hyaluronic acid, heparan sulfate, chondroitin sulfate, and combinations thereof. In some embodiments, an nHC-HA/PTX3 or rcHC-HA/PTX3 complex disclosed herein is formulated with a cross-linked polymer. In some embodiments, an nHC-HA/PTX3 or rcHC-HA/PTX3 complex disclosed herein is formulated with a non-cross-linked polymer. In some embodiments, an nHC-HA/PTX3 or rcHC-HA/PTX3 complex disclosed herein is formulated with a non-cross-linked polymer and a cross-linked polymer. In some embodiments, an nHC-HA/PTX3 or rcHC-HA/PTX3 complex disclosed herein is formulated with cross-linked hyaluronan gel. In some embodiments, an nHC-HA/PTX3 or rcHC-HA/PTX3 complex disclosed herein is formulated with an insoluble cross-linked HA hydrogel. In some embodiments, an nHC-HA/PTX3 or rcHC-HA/PTX3 complex disclosed herein is formulated with non-cross-linked hyaluronan gel. In some embodiments, an nHC-HA/PTX3 or rcHC-HA/PTX3 complex disclosed herein is formulated with a collagen matrix. In some embodiments, an nHC-HA/PTX3 or rcHC-HA/PTX3 complex disclosed herein is formulated with a fibrin matrix. In some embodiments, an nHC-HA/PTX3 or rcHC-HA/PTX3 complex disclosed herein is formulated with a fibrin/collagen matrix.

In some embodiments, an nHC-HA/PTX3 or rcHC-HA/PTX3 complex disclosed herein is formulated for administration to a tumor or a tissue related thereto. Formulations suitable for administration to a tumor include, but are not limited to, solutions, suspensions (e.g., an aqueous suspension), ointments, gels, creams, liposomes, niosomes, pharmacosomes, nanoparticles, or combinations thereof. In some embodiments, an nHC-HA/PTX3 or rcHC-HA/PTX3 complex disclosed herein for injection into a solid tumor is administered by injection into a tumor, the surrounding tissue, or a combination thereof. In some embodiments, an nHC-HA/PTX3 or rcHC-HA/PTX3 complex disclosed herein is administered concurrent to excision of cancer cells or a tumor. In some embodiments, an nHC-HA/PTX3 or rcHC-HA/PTX3 complex disclosed herein is administered to surgical margins from the excision of cancer cells or a tumor. In some embodiments, an nHC-HA/PTX3 or rcHC-HA/PTX3 complex disclosed herein is administered

As used herein, a “depot preparation” is a controlled-release formulation that is implanted in a tumor or a tissue related thereto (e.g., a surgical margin) (for example subcutaneously, intramuscularly, intravitreally, or within the subconjunctiva). In some embodiments, a depot preparation is formulated by forming microencapsulated matrices (also known as microencapsulated matrices) of an nHC-HA/PTX3 or rcHC-HA/PTX3 complex disclosed herein in biodegradable polymers. In some embodiments, a depot preparation is formulated by entrapping an nHC-HA/PTX3 or rcHC-HA/PTX3 complex disclosed herein in liposomes or microemulsions.

In some embodiments, an nHC-HA/PTX3 or rcHC-HA/PTX3 complex disclosed herein is formulated for rectal or vaginal administration. In some embodiments, an nHC-HA/PTX3 or rcHC-HA/PTX3 complex disclosed herein is administered as a suppository. In some embodiments, a composition suitable for rectal administration is prepared by mixing an nHC-HA/PTX3 or rcHC-HA/PTX3 complex disclosed herein with a suitable non-irritating excipient which is solid at ordinary temperatures but liquid at the rectal temperature and will therefore melt in the rectum to release the drug. In some embodiments, a composition suitable for rectal administration is prepared by mixing an nHC-HA/PTX3 or rcHC-HA/PTX3 complex disclosed herein with cocoa butter, glycerinated gelatin, hydrogenated vegetable oils, mixtures of polyethylene glycols of various molecular weights or fatty acid esters of polyethylene glycol.

In certain embodiments, an nHC-HA/PTX3 or rcHC-HA/PTX3 complex described herein is optionally incorporated within controlled release particles, lipid complexes, liposomes, nanoparticles, microspheres, microparticles, nanocapsules or other agents which enhance or facilitate localized delivery to the skin. An example of a conventional microencapsulation process for pharmaceutical preparations is described in U.S. Pat. No. 3,737,337, incorporated herein by reference for such disclosure.

Dosages

The amount of pharmaceutical compositions administered is dependent in part on the individual being treated. In instances where pharmaceutical compositions are administered to a human subject, the daily dosage will normally be determined by the prescribing physician with the dosage generally varying according to the age, sex, diet, weight, general health and response of the individual, the severity of the individual's symptoms, the precise disease or condition being treated, the severity of the disease or condition being treated, time of administration, route of administration, the disposition of the composition, rate of excretion, drug combination, and the discretion of the prescribing physician.

In some instances, an nHC-HA/PTX3 or rcHC-HA/PTX3 complex disclosed herein is administered by local injection, directly into a tumor and/or the surrounding tissue. In some instances, the tumor cannot be removed surgically, or is “inoperable”. In some embodiments, an inoperable tumor is not accessible or the patient has medical conditions that limit the ability to withstand surgery. In some instances, a tumor is in a sensitive location, such as the spinal cord, the brain, or other tissues, where surgical removal could critically damage surrounding tissue. In some instances, a tumor infiltrates or invades surrounding tissue, for example with certain brain cancers, and are impossible to surgically extract without harming the surrounding tissue. Inoperable tumors can arise from, without limitation, central nervous system (CNS) cancer, such as glioblastoma multiforme, breast cancer, pancreatic cancer, or bladder cancer. In some instances, a cancer has multiple secondary tumors, or metastases, elsewhere in the body. The number of secondary tumors may be too great to remove safely. Types of metastatic cancer include, without limitation, bladder cancer, breast cancer, colon cancer, kidney cancer, lung cancer, melanoma, ovarian cancer, pancreatic cancer, prostate cancer, rectal cancer, stomach cancer, thyroid cancer, liver cancer, or uterine cancer.

In some embodiments, the administered dosage of an nHC-HA/PTX3 or rcHC-HA/PTX3 complex disclosed herein is between about 0.001 to about 1000 mg. In some embodiments, the amount of nHC-HA/PTX3 or rcHC-HA/PTX3 complex administered is in the range of about 0.5 to about 50 mg. In some embodiments, the amount of nHC-HA/PTX3 or rcHC-HA/PTX3 complex administered is about 0.001 to about 7 g. In some embodiments, the amount of nHC-HA/PTX3 or rcHC-HA/PTX3 complex administered is about 0.01 to about 7 g. In some embodiments, the amount of nHC-HA/PTX3 or rcHC-HA/PTX3 complex administered is about 0.02 to about 5 g. In some embodiments, the amount of nHC-HA/PTX3 or rcHC-HA/PTX3 complex disclosed herein is about 0.05 to about 2.5 g. In some embodiments, the amount of nHC-HA/PTX3 or rcHC-HA/PTX3 administered is about 0.1 to about 1 g.

In some embodiments, an nHC-HA/PTX3 or rcHC-HA/PTX3 complex disclosed herein is administered, before, during or after the occurrence of a disease or condition. In some embodiments, a combination therapy is administered before, during or after the occurrence of a disease or condition. In some embodiments, an nHC-HA/PTX3 or rcHC-HA/PTX3 complex disclosed herein is administered with a combination therapy before, during or after the occurrence of a disease or condition. In some embodiments, the timing of administering the composition containing an nHC-HA/PTX3 or rcHC-HA/PTX3 disclosed herein varies. Thus, in some examples, an nHC-HA/PTX3 or rcHC-HA/PTX3 complex disclosed herein is used as a prophylactic and is administered continuously to subjects with a propensity to develop conditions or diseases in order to prevent the occurrence of the disease or condition. In some embodiments, an nHC-HA/PTX3 or rcHC-HA/PTX3 complex disclosed herein is administered to a subject during or as soon as possible after the onset of the symptoms. In some embodiments, the administration of an nHC-HA/PTX3 or rcHC-HA/PTX3 complex disclosed herein is initiated within the first 48 hours of the onset of the symptoms, preferably within the first 48 hours of the onset of the symptoms, more preferably within the first 6 hours of the onset of the symptoms, and most preferably within 3 hours of the onset of the symptoms. In some embodiments, the initial administration is via any route practical, such as, for example, an intravenous injection, a bolus injection, infusion over 5 minutes to about 5 hours, a pill, a capsule, transdermal patch, buccal delivery, or combination thereof. An nHC-HA/PTX3 or rcHC-HA/PTX3 complex disclosed herein is preferably administered as soon as is practicable after the onset of a disease or condition is detected or suspected, and for a length of time necessary for the treatment of the disease, such as, for example, from about 1 month to about 3 months. In some embodiments, the length of treatment varies for each subject, and the length is determined using the known criteria. In some embodiments, an nHC-HA/PTX3 or rcHC-HA/PTX3 complex disclosed herein or a formulation containing a complex is administered for at least 2 weeks, preferably about 1 month to about 5 years, and more preferably from about 1 month to about 3 years.

In some embodiments, an nHC-HA/PTX3 or rcHC-HA/PTX3 complex disclosed herein is administered in a single dose, once daily. In some embodiments, an nHC-HA/PTX3 or rcHC-HA/PTX3 complex disclosed herein is administered in multiple doses, more than once per day. In some embodiments, an nHC-HA/PTX3 or rcHC-HA/PTX3 complex disclosed herein is administered twice daily. In some embodiments, an nHC-HA/PTX3 or rcHC-HA/PTX3 complex disclosed herein is administered three times per day. In some embodiments, an nHC-HA/PTX3 or rcHC-HA/PTX3 complex is administered four times per day. In some embodiments, an nHC-HA/PTX3 or rcHC-HA/PTX3 complex disclosed herein is administered more than four times per day.

In some embodiments, an nHC-HA/PTX3 or rcHC-HA/PTX3 complex disclosed herein is administered in a single dose. In some embodiments, an nHC-HA/PTX3 or rcHC-HA/PTX3 complex disclosed herein is administered in a single dose in conjunction with a tumor excision, cryoablation, or radiofrequency ablation.

In some embodiments, an nHC-HA/PTX3 or rcHC-HA/PTX3 complex disclosed herein is administered for prophylactic and/or therapeutic treatments. In therapeutic applications, in some embodiments, an nHC-HA/PTX3 or rcHC-HA/PTX3 complex disclosed herein is administered to an individual already suffering from a disease or condition, in an amount sufficient to cure or at least partially arrest the symptoms of the disease or condition. Amounts effective for this use will depend on the severity and course of the disease or condition, previous therapy, the individual's health status, weight, and response to the drugs, and the judgment of the treating physician.

In prophylactic applications, in some embodiments, an nHC-HA/PTX3 or rcHC-HA/PTX3 complex disclosed herein is administered to an individual that is at risk of a particular disorder. Such an amount is defined to be a “prophylactically effective amount or dose.” In such use, the precise amounts also depend on the individual's state of health, weight, and other physical parameters of the individual.

In the case wherein the individual's condition does not improve, upon the doctor's discretion an nHC-HA/PTX3 or rcHC-HA/PTX3 complex disclosed herein is administered chronically, that is, for an extended period of time, including throughout the duration of the individual's life in order to ameliorate or otherwise control or limit the symptoms of the individual's disease or condition.

In some embodiments, in cases where the individual's status does improve, upon the doctor's discretion, an nHC-HA/PTX3 or rcHC-HA/PTX3 complex disclosed herein is administered continuously or the dose of drug being administered is temporarily reduced or temporarily suspended for a certain length of time (i.e., a “drug holiday”). In some embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. In some embodiments the dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the individual's conditions has occurred, a maintenance dose is administered if necessary. In some embodiments, subsequently, the dosage or the frequency of administration, or both, is reduced, as a function of the symptoms, to a level at which the improved disease, disorder or condition is retained. In some embodiments, individuals require intermittent treatment on a long-term basis upon any recurrence of symptoms.

In some embodiments, the pharmaceutical composition described herein is in unit dosage forms suitable for single administration of precise dosages. In unit dosage form, the formulation is divided into unit doses containing appropriate quantities of an nHC-HA/PTX3 or rcHC-HA/PTX3 complex disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. In some embodiments, multiple-dose reclosable containers are used, in which case it is typical to include a preservative in the composition. In some embodiments, formulations for parenteral injection are presented in unit dosage form, which include, but are not limited to ampoules, or in multi dose containers, with an added preservative.

In some embodiments, the area surrounding the tumor is contacted with at least or about 1 microgram (ug), 10 ug, 20 ug, 30 ug, 40 ug, 50 ug, 60 ug, 70 ug, 80 ug, 90 ug, 100 ug, 200 ug, 300 ug, 400 ug, 500 ug, 600 ug, 700 ug, 800 ug, 900 ug, 1000 ug, or more than 1000 ug of the HC-HA/PTX3 complex. In some embodiments, the area surrounding the tumor is contacted with at least or about 1 milligram (mg), 10 mg, 20 mg, 30 mg, 40 mg, 50 mg, 60 mg, 70 mg, 80 mg, 90 mg, 100 mg, 200 mg, 300 mg, 400 mg, 500 mg, or more than 500 mg of the HC-HA/PTX3 complex. In some embodiments, the area surrounding the tumor is contacted with a range of about 1 to 10, 1 to 20, 1 to 40, 1 to 60, 1 to 80, 1 to 100, 1 to 150, 1 to 200, 10 to 20, 10 to 40, 10 to 60, 10 to 80, 10 to 100, 10 to 150, 10 to 200, 20 to 40, 20 to 60, 20 to 80, 20 to 100, 20 to 150, 20 to 200, 40 to 60, 40 to 80, 40 to 100, 40 to 150, 40 to 200, 60 to 80, 60 to 100, 60 to 150, 60 to 200, 80 to 100, 80 to 150, 80 to 200, 100 to 150, 100 to 200, or 150 to 200 microgram (ug) of the HC-HA/PTX3 complex. In some embodiments, the area surrounding the tumor is contacted with a range of about 1 to 10, 1 to 20, 1 to 40, 1 to 60, 1 to 80, 1 to 100, 1 to 150, 1 to 200, 10 to 20, 10 to 40, 10 to 60, 10 to 80, 10 to 100, 10 to 150, 10 to 200, 20 to 40, 20 to 60, 20 to 80, 20 to 100, 20 to 150, 20 to 200, 40 to 60, 40 to 80, 40 to 100, 40 to 150, 40 to 200, 60 to 80, 60 to 100, 60 to 150, 60 to 200, 80 to 100, 80 to 150, 80 to 200, 100 to 150, 100 to 200, or 150 to 200 milligram (mg) of the HC-HA/PTX3 complex.

The daily dosages appropriate for an nHC-HA/PTX3 or rcHC-HA/PTX3 complex disclosed herein are, for example, from about 0.01 to 100 mg. An indicated daily dosage in the larger mammal, including, but not limited to, humans, is in the range from about 10 ug to about 100 mg from about 0.5 mg to about 100 mg, conveniently administered in divided doses, including, but not limited to, up to four times a day or in extended release form. Suitable unit dosage forms for oral administration include from about 1 to 50 mg active ingredient. Suitable doses for injection into a tumor and/or surrounding tissues is in the range from about 0.1 to about 100 mg per injection. The foregoing ranges are merely suggestive, as the number of variables in regard to an individual treatment regime is large, and considerable excursions from these recommended values are not uncommon. In some embodiments, the dosages are altered depending on a number of variables, not limited to the activity of an nHC-HA/PTX3 or rcHC-HA/PTX3 complex used, the disease or condition to be treated, the mode of administration, the requirements of the individual subject, the severity of the disease or condition being treated, and the judgment of the practitioner.

In some embodiments, the toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). In some embodiments, the dose ratio between the toxic and therapeutic effects is the therapeutic index and it is expressed as the ratio between LD₅₀ and ED₅₀. nHC-HA/PTX3 or rcHC-HA/PTX3 complexes exhibiting high therapeutic indices are preferred. In some embodiments, the data obtained from cell culture assays and animal studies is used in formulating a range of dosage for use in human. The dosage of an nHC-HA/PTX3 or rcHC-HA/PTX3 complex disclosed herein lies preferably within a range of circulating concentrations that include the ED₅₀ with minimal toxicity. In some embodiments, the dosage varies within this range depending upon the dosage form employed and the route of administration utilized.

In some embodiments, the pharmaceutical compositions of nHC-HA/PTX3 or rcHC-HA/PTX3 complexes are packaged as articles of manufacture containing packaging material, a pharmaceutical composition which is effective for prophylaxis and/or treating a disease or condition, and a label that indicates that the pharmaceutical composition is to be used for treating the disease or condition. In some embodiments, the pharmaceutical compositions are packaged in unit dosage forms contain an amount of the pharmaceutical composition for a single dose or multiple doses. In some embodiments, the packaged compositions contain a lyophilized powder of the pharmaceutical compositions, which is reconstituted (e.g., with water or saline) prior to administration.

Medical Device

In some embodiments, an nHC-HA/PTX3 or rcHC-HA/PTX3 complex disclosed herein is assembled directly on a surface of or formulated as a coating for an implantable medical device. Methods for covalent attachment of hyaluronan to surfaces such as, but not limited to, metallic, polymeric, ceramic, silica and composite surfaces is well-known in the art and in some embodiments, is employed in conjunction with the methods provided herein for the assembly of nHC-HA/PTX3 or rcHC-HA/PTX3 complexes on such surfaces (see e.g., U.S. Pat. Nos. 5,356,433; 5,336,518, 4,613,665, 4,810,784, 5,037,677, 8,093,365). In some embodiments, an nHC-HA/PTX3 or rcHC-HA/PTX3 complex is assembled directly on a surface of an implantable medical device or a portion thereof. In some embodiments, an nHC-HA/PTX3 or rcHC-HA/PTX3 complex that has been generated according the methods provided herein is purified and then attached directly on a surface of an implantable medical device or a portion thereof. In some embodiments, an nHC-HA/PTX3 or rcHC-HA/PTX3 complex that has been generated according the methods provided herein is purified and then formulated as a coating for attachment to the medical device or a portion thereof. In some embodiments, the coating is applied directly to the surfaces or is applied to a pretreated or coated surface where the pretreatment or coating is designed to aid adhesion of the coating to the substrate. In some embodiments, an nHC-HA/PTX3 or rcHC-HA/PTX3 complex that has been generated according the methods provided herein is purified and then attached to a medical device or a portion thereof that has been coated with a substance that promotes the attachment of the nHC-HA/PTX3 or rcHC-HA/PTX3 complex. For example, in some embodiments, the medical device or a portion thereof is coated with an adhesive polymer that provides functional groups on its surface for the covalent attachment of hyaluronan of the nHC-HA/PTX3 or rcHC-HA/PTX3 complex. In some embodiments, a coupling agent, such as, but not limited to carbodiimide is employed to attach the nHC-HA/PTX3 or rcHC-HA/PTX3 complex to the polymer coating. In some embodiments, photoimmobilization is employed to covalently attach an nHC-HA/PTX3 or rcHC-HA/PTX3 complex that has been generated according the methods provided herein to medical device or a portion thereof. In some embodiments, an nHC-HA/PTX3 or rcHC-HA/PTX3 complex that has been generated according the methods provided herein is attached to a medical device or a portion thereof using a spacer molecule that comprises a photochemically or thermochemically reactive group.

In some embodiments, the coating formulations comprising an nHC-HA/PTX3 or rcHC-HA/PTX3 complex are applied to the substrate by for example dip-coating. Other methods of application include, but are not limited to, spray, wash, vapor deposition, brush, roller, curtain, spin coating and other methods known in the art.

Exemplary implantable medical devices include, but are not limited to a bone implant, wound drain, shunt, urethral insert, metal or plastic implant, stent, stent graft, vascular graft, pellets, wafers, implantable drug pump, drug delivery system, microparticle, nanoparticle, and microcapsule.

In some embodiments, an nHC-HA/PTX3 or rcHC-HA/PTX3 complex disclosed herein is attached to the microcapsule or assembled directly on a microcapsule. In some embodiments, the nHC-HA/PTX3 or rcHC-HA/PTX3 complex is combined with a material used to form the microcapsule and a microcapsule is generated that contains the nHC-HA/PTX3 or rcHC-HA/PTX3 complex. In some embodiments, the nHC-HA/PTX3 or rcHC-HA/PTX3 complex is used to coat the inner surface of the microcapsule. In some embodiments, the nHC-HA/PTX3 or rcHC-HA/PTX3 complex is used to coat the outer surface of the microcapsule. In some embodiments, the nHC-HA/PTX3 or rcHC-HA/PTX3 complex is used to coat the inner and outer surface of the microcapsule.

### Combinations

In some embodiments, the compositions and methods described herein are used in conjunction with a second or further or additional therapeutic agent in addition to the native or reconstituted HC-HA/PTX3 complex. In some embodiments, the compositions and methods described herein are used in conjunction with two or more therapeutic agents. In some embodiments, the compositions and methods described herein are used in conjunction with one or more therapeutic agents. In some embodiments, the compositions and methods described herein are used in conjunction with 2, 3, 4, 5, 6, 7, 8, 9, 10 or more therapeutic agents.

In some embodiments, an nHC-HA/PTX3 or rcHC-HA/PTX3 complex disclosed herein and a second therapeutic agent are administered in the same dosage form. In some embodiments, an nHC-HA/PTX3 or rcHC-HA/PTX3 complex disclosed herein and a second therapeutic agent are administered in separate dosage forms.

In some embodiments, an nHC-HA/PTX3 or rcHC-HA/PTX3 complex disclosed herein and a second therapeutic agent are administered concurrently (e.g., simultaneously, essentially simultaneously or within the same treatment protocol).

In some embodiments, an nHC-HA/PTX3 or rcHC-HA/PTX3 complex disclosed herein and a second therapeutic agent are administered sequentially. In some embodiments, an nHC-HA/PTX3 or rcHC-HA/PTX3 complex disclosed herein is administered before or after the second therapeutic agent. In some embodiments, the time period between administration of an nHC-HA/PTX3 or rcHC-HA/PTX3 complex disclosed herein and a second active agent ranges from a few minutes to several hours, depending upon the properties of each pharmaceutical agent, such as potency, solubility, bioavailability, plasma half-life and kinetic profile of the pharmaceutical agent. In some embodiments, circadian variation of the target molecule concentration determines the optimal dose interval. In some embodiments, the timing between the administration of an nHC-HA/PTX3 or rcHC-HA/PTX3 complex disclosed herein and a second active agent is about an hour, about 2 hours, about 3 hours, about 4 hours, about 5 hours, about 6 hours, about 7 hours, about 8 hours, about 9 hours, about 10 hours, about 11 hours, about a day, about 2 days, about 3 days, about 4 days, about 5 days, about 6 days, about a week, about 2 weeks, about 3 weeks, about a month, or longer.

In some embodiments, the co-administration of an nHC-HA/PTX3 or rcHC-HA/PTX3 complex disclosed herein results in a lower required dosage for the nHC-HA/PTX3 or rcHC-HA/PTX3 complex than the required dosage when administering an nHC-HA/PTX3 or rcHC-HA/PTX3 complex alone. In some embodiments, the co-administration of a second therapeutic agent results in a lower required dosage for the second agent than the required dosage when administering the second agent alone. Methods for experimentally determining therapeutically-effective dosages of drugs and other agents for use in combination treatment regimens are known and described in the art. For example, the use of metronomic dosing, i.e., providing more frequent, lower doses in order to minimize toxic side effects, has been described extensively in the art. Combination treatment further includes periodic treatments that start and stop at various times to assist with the clinical management of the individual.

In some embodiments, the combination treatment nHC-HA/PTX3 or rcHC-HA/PTX3 complex and one or more additional therapeutic agents is modified. In some embodiments, the combination treatment is modified, whereby the amount of the nHC-HA/PTX3 or rcHC-HA/PTX3 complex is increased relative to the amount of a second therapeutic agent. In some embodiments, the combination treatment is modified, whereby the amount of the nHC-HA/PTX3 or rcHC-HA/PTX3 complex is decreased relative to the amount of a second therapeutic agent. In some embodiments, the combination treatment is modified, whereby the amount of a second therapeutic agent increased relative to the amount of the nHC-HA/PTX3 or rcHC-HA/PTX3 complex. In some embodiments, the combination treatment is modified, whereby the amount of a second therapeutic agent decreased relative to the amount of the nHC-HA/PTX3 or rcHC-HA/PTX3 complex.

In some embodiments, the second therapeutic agent is selected from cytotoxic agents, analgesics, anti-inflammatories, antibiotics, antimicrobial agents, anti-angiogenesis agents, chemotherapeutic agents, anti-neoplastic agents, immunotherapy, or radiation therapy. In some embodiments the second therapeutic agent is a chemotherapeutic agent. In some embodiments, the second therapeutic agent is selected from alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, hematopoietic growth factors, aromatase inhibitors, anti-estrogens, anti-androgens, corticosteroids, gonadorelin agonists, microtubule active agents, nitrosoureas, lipid or protein kinase targeting agents, immunomodulatory drugs (IMiDs), protein or lipid phosphatase targeting agents, anti-angiogenic agents, Akt inhibitors, IGF-I inhibitors, FGF3 modulators, mTOR inhibitors, Smac mimetics, HDAC inhibitors, agents that induce cell differentiation, bradykinin 1 receptor antagonists, angiotensin II antagonists, cyclooxygenase inhibitors, heparanase inhibitors, lymphokine inhibitors, cytokine inhibitors, IKK inhibitors, P38MAPK inhibitors, HSP90 inhibitors, multikinase inhibitors, bisphosphonate, rapamycin derivatives, anti-apoptotic pathway inhibitors, apoptotic pathway agonists, PPAR agonists, RAR agonists, inhibitors of Ras isoforms, telomerase inhibitors, protease inhibitors, metalloproteinase inhibitors, aminopeptidase inhibitors, SHIP activators—AQX-MN100, Humax-CD20 (ofatumumab), CD20 antagonists, IL2-diptheria toxin fusions, or combinations thereof. In some embodiments, the antimicrobial agent is an antiviral, antibacterial or antifungal agent. Non-limiting exemplary antibacterial agent(s) include those classified as aminoglycosides, beta lactams, quinolones or fluoroquinolones, macrolides, sulfonamides, sulfamethaxozoles, tetracyclines, streptogramins, oxazolidinones (such as linezolid), clindamycins, lincomycins, rifamycins, glycopeptides, polymxins. lipo-peptide antibiotics, as well as pharmacologically acceptable sodium salts, pharmacologically acceptable calcium salts, pharmacologically acceptable potassium salts, lipid formulations, derivatives and/or analogs of the above. Some exemplary classes of innate peptides or proteins are transferrins, lactoferrins, defensins, phospholipases, lysozyme, cathelicidins, serprocidins, bacteriocidal permeability increasing proteins, amphipathic alpha helical peptides, and other synthetic antimicrobial proteins. In some embodiments, the antimicrobial agent is an antiseptic agent.

In some embodiments, the second therapeutic agent is selected from ARRY-797, dacarbazine (DTIC), actinomycins C₂, C₃, D, and F₁, cyclophosphamide, melphalan, estramustine, maytansinol, rifamycin, streptovaricin, doxorubicin, daunorubicin, epirubicin, idarubicin, detorubicin, carminomycin, esorubicin, mitoxantrone, bleomycins A, A₂, and B, camptothecin, Irinotecan, Topotecan, 9-aminocamptothecin, 10,11-methylenedioxycamptothecin, 9-nitrocamptothecin, bortezomib, temozolomide, TAS103, NPI0052, combretastatin, combretastatin A-2, combretastatin A-4, calicheamicins, neocarcinostatins, epothilones A B, C, and semi-synthetic variants, Herceptin, Rituxan, CD40 antibodies, asparaginase, interleukins, interferons, leuprolide, and pegaspargase, 5-fluorouracil, fluorodeoxyuridine, ptorafur, 5′-deoxyfluorouridine, UFT, MITC, S-1 capecitabine, diethylstilbestrol, tamoxifen, toremefine, tolmudex, thymitaq, flutamide, fluoxymesterone, bicalutamide, finasteride, estradiol, trioxifene, dexamethasone, leuproelin acetate, estramustine, droloxifene, medroxyprogesterone, megesterol acetate, aminoglutethimide, testolactone, testosterone, diethylstilbestrol, hydroxyprogesterone, mitomycins A, B and C, porfiromycin, cisplatin, carboplatin, oxaliplatin, tetraplatin, platinum-DACH, ormaplatin, thalidomide, lenalidomide, CI-973, telomestatin, CHIR258, Rad 001, SAHA, Tubacin, 17-AAG, sorafenib, JM-216, podophyllotoxin, epipodophyllotoxin, etoposide, teniposide, Tarceva, Iressa, Imatinib, Miltefosine, Perifosine, aminopterin, methotrexate, methopterin, dichloro-methotrexate, 6-mercaptopurine, thioguanine, azathioprine, allopurinol, cladribine, fludarabine, pentostatin, 2-chloroadenosine, deoxycytidine, cytosine arabinoside, cytarabine, azacitidine, 5-azacytosine, gencitabine, 5-azacytosine-arabinoside, vincristine, vinblastine, vinorelbine, leurosine, leurosidine and vindesine, paclitaxel, taxotere and/or docetaxel.

In some embodiments, the second therapeutic agent is niacin, a fibrate, a statin, a Apo-A1 mimetic polypeptide (e.g., DF-4, Novartis), an apoA-I transcriptional up-regulator, an ACAT inhibitor, a CETP modulator, Glycoprotein (GP) IIb/IIIa receptor antagonists, P2Y12 receptor antagonists, Lp-PLA2-inhibitors, an anti-tumor necrosis factor (TNF) agent, an interleukin-1 (IL-1) receptor antagonist, an interleukin-2 (IL-2) receptor antagonist, an interleukin-6 (IL-6) receptor antagonist, an interleukin-12 (IL-12) receptor antagonist, an interleukin-17 (IL-17) receptor antagonist, an interleukin-23 (IL-23) receptor antagonist, a cytotoxic agent, an antimicrobial agent, an immunomodulatory agent, an antibiotic, a T-cell co-stimulatory blocker, a B cell depleting agent, an immunosuppressive agent, an anti-lymphocyte antibody, an alkylating agent, an anti-metabolite, a plant alkaloid, a terpenoids, a topoisomerase inhibitor, an anti-tumor antibiotic, a monoclonal antibody, a hormonal therapy (e.g., aromatase inhibitors), or combinations thereof.

In some embodiments, the second active agent is an anti-TGF-β antibody, an anti-TGF-β receptor blocking antibody, an anti-TNF antibody, an anti-TNF receptor blocking antibody, an anti-IL1β antibody, an anti-IL1β receptor blocking antibody, an anti-IL-2 antibody, an anti-IL-2 receptor blocking antibody, an anti-IL-6 antibody, an anti-IL-6 receptor blocking antibody, an anti-IL-12 antibody, an anti-IL-12 receptor blocking antibody, an anti-IL-17 antibody, anti-IL-17 receptor blocking antibody, an anti-IL-23 antibody, or an anti-IL-23 receptor blocking antibody.

In some embodiments, the second active agent is alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, sulfasalazine, etanercept, adalimumab, infliximab, abatacept, rituximab, trastuzumab, anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Betal Monoclonal Antibody, Genzyme), antibody 3F8 (NCI), 177Lu-J591 (Weill Medical College of Cornell University), TB-403 (BioInvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine, AIN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1beta monoclonal antibody, Novartis), CNTO 1275 (Fully Human Anti-IL-12 Monoclonal Antibody, Centocor), (3S)-N-hydroxy-4-({4-[(4-hydroxy-2-butynyl)oxy]phenyl}sulfonyl)-2,2-dimet- hyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca),), [(1R)-3-methyl-1-({2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA (Tocilizumab, an Anti-IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol; cisplatin; carboplatin; oxaliplatin; mechlorethamine; cyclophosphamide; chlorambucil; vincristine; vinblastine; vinorelbine; vindesine; azathioprine; mercaptopurine; fludarabine; pentostatin; cladribine; 5-fluorouracil (5FU); floxuridine (FUDR); cytosine arabinoside; methotrexate; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; trastuzumab; cetuximab; rituximab; bevacizumab; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; or combinations thereof.

In some embodiments, the second therapeutic agent is an antibiotic. In some embodiments, the second therapeutic agent is an anti-bacterial agent. In some embodiments, the second therapeutic agent is amikacin, gentamicin, kanamycin, neomycin, netilmicin, streptomycin, tobramycin, paromomycin, geldanmycin, herbimycin, loracarbef, ertapenem, doripenem, imipenem, cilastatin, meropenem, cefadroxil, cefazolin, cefalotin, cefalexin, cefaclor, cefamandole, cefoxitin, defprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillan, bacitracin, colistin, polymyxin B, ciprofloxacin, enoxacin, gatifloxacin, levofloxacin, lomefloxacin, moxifloxacin, norfloxacin, ofloxacin, trovfloxacin, mafenide, prontosil, sulfacetamide, sulfamethizole, sulfanimilimde, sulfsalazine, sulfsioxazole, trimethoprim, demeclocycline, doxycycline, minocycline, oxtetracycline, tetracycline, arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinuspristin/dalfopristin, rifampin, tinidazole, and combinations thereof.

In some embodiments, the second therapeutic agent is a radiation therapy. In some embodiments, the second therapeutic agent is selected from x-ray therapy or proton beam therapy. In some embodiments, the radiation therapy can be external beam radiation or brachytherapy.

In some embodiments, the second therapeutic agent is a targeted therapy. A targeted therapy targets specific genes, proteins or tissue environment that contributes to cancer growth and survival. In some embodiments, a targeted therapy comprises one or more monoclonal antibodies. In some embodiments, a targeted therapy comprises small molecules, for example, without limitation, angiogenesis inhibitors, as described herein. In some embodiments an targeted therapy comprises one or more monoclonal antibodies and one or more small molecules, as described herein.

Combinations with Cells

In some embodiments, an nHC-HA/PTX3 or rcHC-HA/PTX3 complex disclosed herein is co-administered with a cell, a plurality of cells or a tissue.

In some embodiments, an nHC-HA/PTX3 or rcHC-HA/PTX3 complex disclosed herein is co-administered with a therapeutic cell. In some embodiments, an nHC-HA/PTX3 or rcHC-HA/PTX3 complex disclosed herein is co-administered with a tissue transplant. In some embodiments, an nHC-HA/PTX3 or rcHC-HA/PTX3 complex disclosed herein is co-administered with a stem cell transplant. In some embodiments, an nHC-HA/PTX3 or rcHC-HA/PTX3 complex disclosed herein is co-administered with an organ transplant. In some embodiments, an nHC-HA/PTX3 or rcHC-HA/PTX3 complex disclosed herein is co-administered with immune cells.

In some embodiments, an nHC-HA/PTX3 or rcHC-HA/PTX3 complex disclosed herein is administered concurrently (e.g., simultaneously, essentially simultaneously or within the same treatment protocol) with a tumor excision, cryoablation, or radiofrequency ablation. In some embodiments, an nHC-HA/PTX3 or rcHC-HA/PTX3 complex disclosed herein is administered before or after a tumor excision, cryoablation, or radiofrequency ablation. In some embodiments, the time period between administration of an nHC-HA/PTX3 or rcHC-HA/PTX3 complex disclosed herein and the tumor excision, cryoablation, or radiofrequency ablation ranges from a few minutes to several hours, depending upon the properties of each pharmaceutical agent, such as potency, solubility, bioavailability, plasma half-life and kinetic profile of the pharmaceutical agent. In some embodiments, circadian variation of the target molecule concentration determines the optimal dose interval. In some embodiments, the timing between the administration of an nHC-HA/PTX3 or rcHC-HA/PTX3 complex disclosed herein and a second active agent is about less than an hour, less than a day, less than a week, or less than a month.

In some embodiments, an nHC-HA/PTX3 or rcHC-HA/PTX3 complex disclosed herein is co-administered with a tumor excision, cryoablation, or radiofrequency ablation and an immunosuppressive agent. In some embodiments, an nHC-HA/PTX3 or rcHC-HA/PTX3 complex disclosed herein is co-administered with a tumor excision, cryoablation, or radiofrequency ablation and a calcineurin inhibitor (e.g., cyclosporin or tacrolimus); an mTOR inhibitor (sirolimus; everolimus); an anti-proliferative agent (azathioprine or mycophenolic acid); a corticosteroid (e.g., prednisolone or hydrocortisone); a monoclonal anti-IL-2Rα receptor antibody (e.g., basiliximab or daclizumab); polyclonal anti-T-cell antibodies (e.g., anti-thymocyte globulin (ATG) or anti-lymphocyte globulin (ALG)); chemotherapeutic; an analgesic; an anti-inflammatory; a steroid; and an antibiotic or combinations thereof.

In some embodiments, a tissue is coated with an nHC-HA/PTX3 or rcHC-HA/PTX3 complex disclosed herein. In some embodiments, a plurality of stem cells are coated with an nHC-HA/PTX3 or rcHC-HA/PTX3 complex disclosed herein. In some embodiments, an organ is coated with an nHC-HA/PTX3 or rcHC-HA/PTX3 complex disclosed herein. In some embodiments, coating a tissue with an nHC-HA/PTX3 or rcHC-HA/PTX3 complex disclosed herein prevent a tissue from being acted upon by the host immune system.

In some embodiments, an organ, tissue, or plurality of stem cells is contacted with an nHC-HA/PTX3 or rcHC-HA/PTX3 complex disclosed herein. In some embodiments, an organ, tissue, or plurality of stem cells is contacted with a composition comprising an nHC-HA/PTX3 or rcHC-HA/PTX3 complex disclosed herein. In some embodiments, the composition has a pH of about 7.0 to about 7.5. In some embodiments, the composition has a pH of 7.4. In some embodiments, the composition further comprises potassium, magnesium, and raffinose. In some embodiments, the composition further comprises at least one of adenosine, glutathione, allopurinol, and hydroxyethyl starch. In some embodiments, the composition is UW solution supplemented with an nHC-HA/PTX3 or rcHC-HA/PTX3 complex disclosed herein.

In some embodiments, the organ, tissue, or plurality of stem cells are contacted with an nHC-HA/PTX3 or rcHC-HA/PTX3 complex disclosed herein for about 30 minutes, about 1 hour, about 2 hours, about 3 hours, about 4 hours, about 5 hours, about 6 hours, about 12 hours, about 24 hours, about 36 hours, or about 48 hours. In some embodiments, the contacting occurs at a temperature that protects tissues and vascular conditioning (e.g., less than ambient temperature). In some embodiments, the contacting occurs at 4° C.

In some embodiments, an nHC-HA/PTX3 or rcHC-HA/PTX3 complex disclosed herein is co-administered with immune cells to a subject in need thereof. In some embodiments, the immune cells are allogenic to the subject in need thereof. In some embodiments, the immune cells are autogenic to the subject in need thereof. In some embodiments, the immune cells are genetically modified before administration to a subject in need thereof. In some embodiments, the immune cells are modified to comprise a chimeric antigen receptor (CAR T-cell therapy).

### EXAMPLES

The following examples are included for illustrative purposes only and are not intended to limit the scope of the claimed subject matter.

### Example 1: Determine the Viability and Metabolic Activity of Prostate Cancer Cell-Lines after Exposure to Cryopreserved AM and UC Extracts or Purified HC-HA/PTX3 (AM)

Published data (Alvim, et al., “The potential risk of tumor progression after use of dehydrated human amnion/chorion membrane allograft in a positive margin resection model”, Ther Adv Uro 2019, vol 11: 1-10) indicates faster tumor growth following partial resection in mice treated with a dehydrated human amniotic and chorionic membrane allograft product compared to those with no treatment. Experiments were conducted to determine the viability and metabolic activity of prostate cancer cell-lines after exposure to cryopreserved AM and UC extracts or purified HC-HA/PTX3 (AM).

Extracts or purified HC-HA/PTX3, following two or more runs of ultracentrifugation, of cryopreserved AM and UC were examined to determine if they inhibit proliferation, and reduce the overall cell metabolic activity of human prostate cancer cell-lines, i.e., PC-3 and LNCaP.

For initial feasibility, the effect of purified HC-HA/PTX3 and refined BTGEL (i.e., morselized AM and UC) using the WST-1 assay was tested. The WST-1 assay was used to quantify the total cell metabolic activity.

### Amplification of LNCaP and PC-3

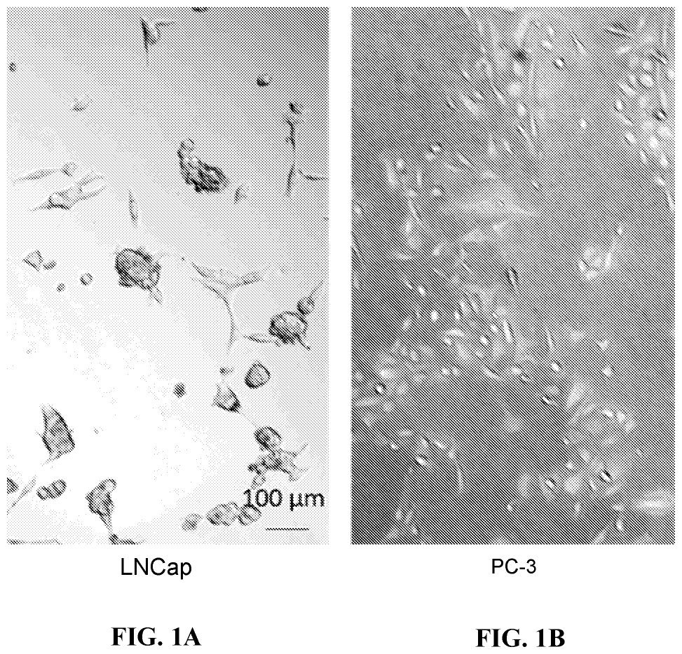

Purchased frozen PC-3 (ATCC® CRL-1435™, lot #70004013) and LNCaP (LNCaP clone FGC ATCC® CRL-1740, lot #64207637) were received and stored in liquid nitrogen. Each vial (1 ml, amount of cells is not provided) of LNCaP and PC-3 was centrifuged at 200×g for 5 minutes. The storage medium was removed, and cells were resuspended into 1 ml RPMI 1640 complete medium. A total of 0.5 ml was transferred to a 100 mm dish (two dishes for each cell line). Cells were incubated at 37° C. with 5% CO₂. The medium was replaced after three days and cells were continued to incubate for four days, when cells were 70% confluency. It was noted that LNCaP tended to form aggregates (FIG. 1A) while PC-3 grew as an evenly distributed single cell layer (FIG. 1B). Cells were harvested by 0.25% Trypsin-EDTA (cat #25200-056, Fisher Scientific) and counted by hemocytometer. The total cells of LNCaP and PC-3 were 5.92×10⁶ and 2.88×10⁶, respectively.

Cells were resuspended into RPMI1640 complete medium with 5% DMSO as follows:

LNCaP: 10 tubes, 5.4×10⁵/tube, 0.5 ml per tube (˜1.08×10⁶/ml), liquid nitrogen tank: S1R4B5, and
PC-3: 10 tubes, 2.6×10⁵/tube, 0.5 ml per tube (˜5.2×10⁵/ml), liquid nitrogen tank: S1R4B5.
Effects of rBTGel and HC-HA/PTX3 on Cell Metabolic Activity of LNCaP and PC-3

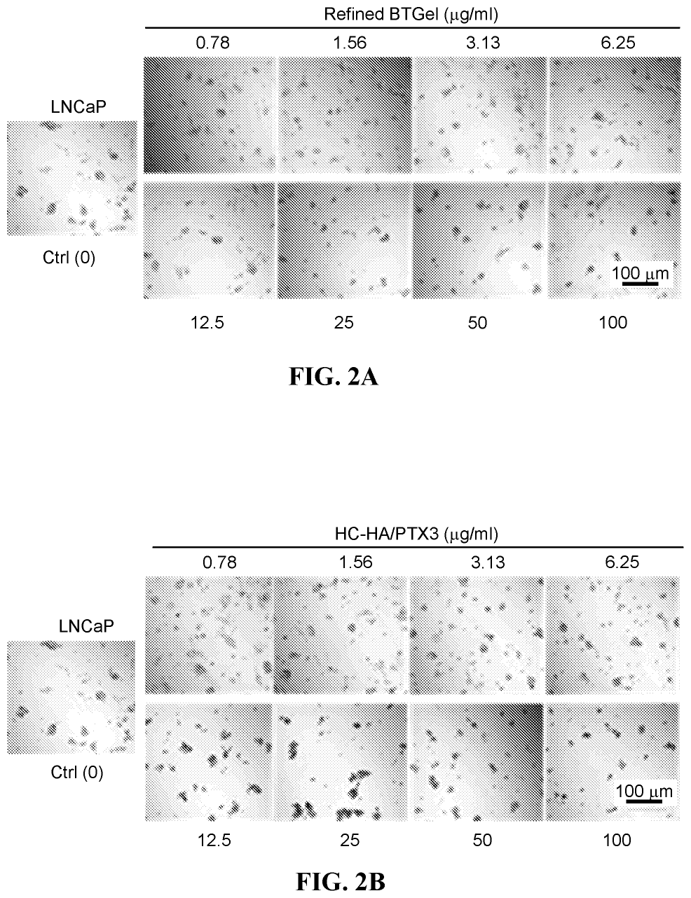

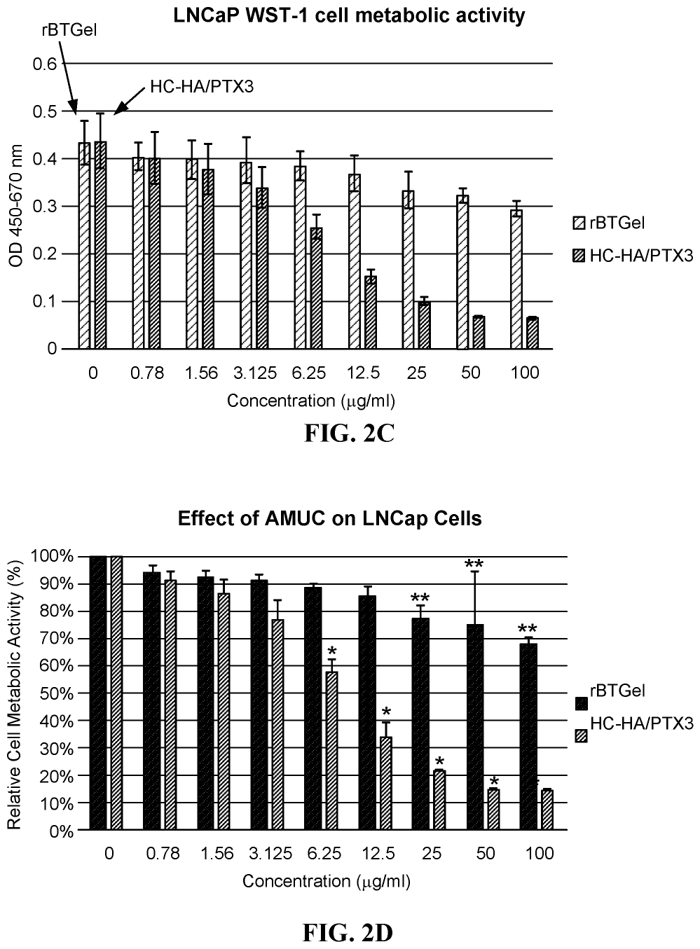

2.9×10⁵ of LNCaP cells or 2.9×10⁵ of PC-3 cells from the amplification (mentioned above) were passaged in one 100 mm dish and cultivated for 4 days. Cells were harvested (LNCaP: total cells were 1.24×10⁶, viability was 91%; PC-3: total cells were 4.08×10⁶, viability was 97%; so PC-3 cells grow faster than LNCaP cells, consistent with the report that the cell doubling times for LNCaP and PC-3 are about 60 hours and 33 hours, respectively) and seeded on a 96-well plate at 1×10⁴/cm² or 3.2×10⁴/ml (one plate for LNCaP and one plate for PC-3). After incubation overnight, both LNCaP and PC-3 cells (n=3) were treated with 0, 0.78, 1.56, 3.125, 6.25, 12.5, 25, 50, or 100 μg/ml of rBTGel (Donor #BTR161857; HA: 175 μg/ml; Protein: 165 μg/ml) or HC-HA/PTX3 (pooled fractions 3-9 of the 2nd ultracentrifugation of AM/PBS, Donor 2 for stability validation, DI: TGLP17E002) (e.g., add 11.1 μl of 10× dose to 100 μl medium to get the exact dose). After treatment for 48 hours, cell morphology (FIG. 2A and 2B, and FIG. 3A and 3B) was recorded by microscope images and then used for WST-1 assay (cat #10008883, Cayman Chemical Company, Ann Arbor, Mich.) for cell metabolic activity according to the manufacturer's instructions (OD450 or OD450-OD670) (FIG. 2C and 2D and FIG. 3C and 3D).

Morphologically, LNCaP grew as small and large cell clusters. After treatment of cells for 48 h, the refined BTgel at higher doses (50 and 100 μg/ml) caused most spindle-like cells to turn into round cells, but most cells were still attached and as clusters. Cell death might have also occurred based on lower cell density. The similar morphological changes occurred at much lower doses of HC-HA/PTX3 (6.25 μg/ml or higher). These data imply that both refined BTGel and HC-HA/PTX3 inhibits cell metabolic activity of LNCaP. The refined BTGel was prepared in saline (˜154 mM NaCl) whereas the HC-HA/PTX3 had been extensively dialyzed with distilled water and contained undetectable salt. Therefore, when lyophilized BTGel was added to the cell culture medium, it may have increased the salt concentration in the medium, and potentially reducing cell proliferation if it increased the salt concentration by ˜30 mM or higher. When the refined BTGel was added as 0.78, 1.56, 3.125, 6.25, 12.5, 25, 50, 100 μg/ml, it increased salt concentration in the culture medium by 6.2, 12.4, 24.7, 49.5, 98.9, 197.9, 395.8, 791.5 mM (use formula dose/175*154/0.111 to get salt concentration; HA concentration of the BTGel is 175 μg/ml, saline contains 154 mM NaCl, the total volume of medium in each well is 0.111 ml), respectively. Thus, the inhibition by the refined BTGel at 6.25 μg/ml or higher may be caused by salt, while there should be no concern of salt effect by HC-HA/PTX3. WST-1 assay data showed the metabolic activity of LNCaP was inhibited (p<0.05) when the dose of the refined BTGel was at 25 μg/ml or higher (FIG. 2C). Because of concern of salt effect, it is inconclusive whether the refined BTGel can inhibit the cell metabolic activity of LNCaP at these doses. In contrast, HC-HA/PTX3 at 6.25 μg/ml or higher inhibits the metabolic activity of LNCaP (by 40-85%) (FIG. 2C and 2D).

Morphologically, PC-3 grew as more evenly distributed cells than the LNCaP. However, after treatment of cells for 48 h, the refined BTGel at higher doses (50 and 100 μg/ml) caused more rounded cells. Cells death might also have occurred based on lower cell density. The similar morphological changes occurred at much lower doses of HC-HA/PTX3 (12.5 μg/ml or higher). These data imply that both refined BTGel and HC-HA/PTX3 inhibits proliferation (viability) of PC-3. However, as mentioned above, the inhibition by the refined BTGel at 6.25 μg/ml or higher may be caused by salt, while there should be no concern of salt effect by HC-HA/PTX3. WST-1 assay data showed the proliferation of PC-3 was inhibited (p<0.05) when the dose of the refined BTGel was at 6.25 μg/ml or higher (FIG. 3C). Because of concern of salt effect, it is inconclusive whether the refined BTGel can inhibit the proliferation of PC-3 at these doses. In contrast, HC-HA/PTX3 at 3.125 μg/ml or higher inhibits the proliferation of PC-3 (by 8-100%) (FIG. 3C and 3D).

Both treatment groups were shown to have a dose-dependent effect to significantly decrease the metabolic effect of PC-3 and LNCaP prostate cancer cells. Based on the morphology, it appears there was cell death associated with the WST-1 readouts for the higher concentrations.

These data showed HC-HA/PTX3 did not promote PC-3 and LNCaP cancer formation, allowing for use post-prostatectomy. Furthermore, rBTGEL was shown to have anti-cancer effects. The following example will address the effect of UC extract without potential salt effects on proliferation of prostate cancer cells.

Conclusion

Refined BTGel at 6.25 μg/ml or higher and HC-HA/PTX3 at 3.125 μg/ml or higher inhibits the proliferation of PC-3. Refined BTGel at 25 μg/ml or higher and HC-HA/PTX3 at 6.25 μg/ml or higher inhibits the proliferation of LNCaP. Because of the concern of salt effect, it is inconclusive whether the amniotic membrane and umbilical cord (AMUC) or salt in the refined BTGel can inhibit the proliferation at these doses.

A lower concentration of rBTGEL and HC-HA/PTX3 may be needed to inhibit PC-3 compared to LNCaP because PC-3 cells are known to have a quicker doubling time. Hence, the difference between the negative control and treatment groups would be greater in PC-3.

Accordingly, even though published data indicates that certain fetal support products are associated with faster tumor relapse and growth, it is demonstrated here that AM and UC products will not promote cancer recurrence after prostatectomy.

### Example 2: Determine the Metabolic Activity of Prostate Cancer Cell-Lines after Exposure to Cryopreserved UC Extracts, HA, and Purified HC-HA/PTX3 (AM)

The results of Example 1 show HC-HA/PTX3 reduces the metabolic activity of both prostate cell lines at concentrations as low as 6.25 μg/ml in both cell types. In addition, rBTGEL was shown to inhibit the activity at 25 μg/ml and above. The following study was performed to rule out the possibility that salt concentration in the higher doses of rBTGel was confounding the data interpretation.

UC extract in water was tested using the same WST-1 assay. In addition, HC-HA/PTX3 was tested to compare to UC results. HA was used as a control group. A series of doses were tested based on the HA μg/ml.

PC3 and LNCaP cells were plated at 1×10⁴/cm² in 96-well plates (100 ul/well) (n=3 for each group). After incubation overnight, cells were treated with a series of doses (defined by HA concentration, 0, 1.56, 3.125, 6.25, 12.5, 25, 50, 100, 200 and 400 μg/ml, n=3) of UC extracted with water, purified HC-HA/PTX3 (pooled fractions 3-9 of the 2nd ultracentrifugation of AM/PB S, Donor 2 for stability validation, DI: TGLP17E002, prepared on Sep. 19, 2017) and HA. After treatment for 48 h, the cell morphology was recorded, and then cells were measured by WST-1 (cat #10008883, Cayman Chemical Company, Ann Arbor, Mich.) for cell metabolic activity according to the manufacturer's instructions (OD450 or OD450-OD670).

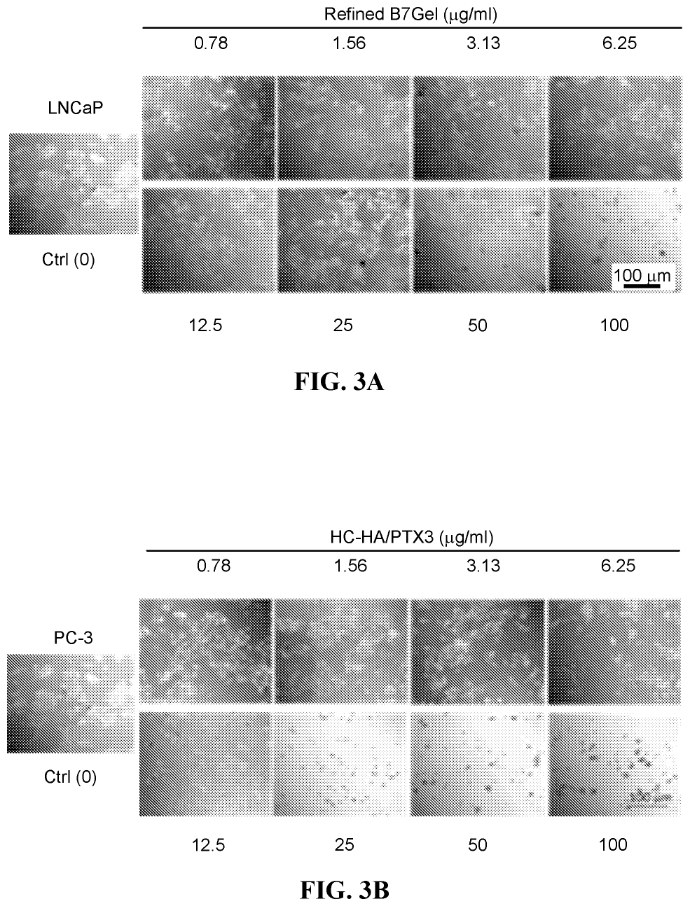

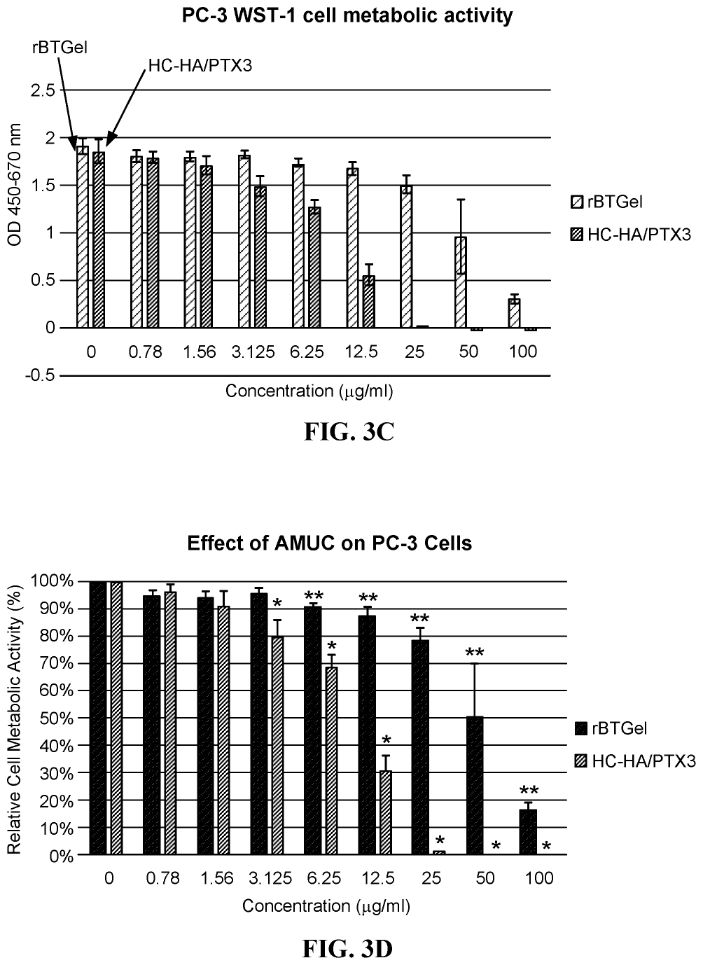

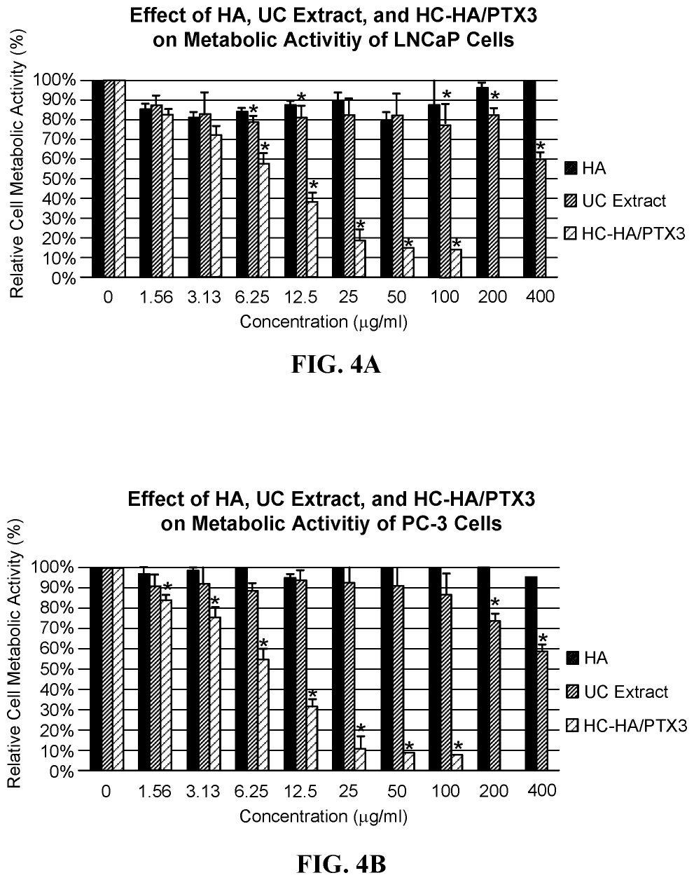

WST-1 assay data showed the metabolic activity of LNCaP cells was significantly inhibited (p<0.05) when treated with UC extract (≥100 μg/ml) and HC-HA/PTX3 (≥6.25 μg/ml) but not HA (See FIG. 4A). Similarly, the metabolic activity in PC-3 cells was significantly inhibited by UC extract (≥200 μg/ml) and HC-HA/PTX3 (≥1.56 μg/ml) but not HA (See FIG. 4B). Cell death was markedly noted with microscopy in both LNCaP (FIGS. 5A-5C) and PC3 (FIGS. 6A-6C) cells with HC-HA/PTX3 at concentrations ≥25 μg/ml.

There was a clear inhibition of metabolic activity by HC-HA/PTX3 as determined by WST-1 and morphological evaluation. Based on morphology, PC3 and LNCaP cells both become rounded, less adherent to the matrix, and there are fewer cells when exposed to higher concentrations of HC-HA/PTX3. A difference is the LNCaP cells aggregate as clusters with small diameter, rounded cells at higher HC-HA/PTX3 concentrations, however the PC3 cells do not aggregate.

WST-1 assay data showed the metabolic activity of LNCaP cells was significantly inhibited (p<0.05) when treated with UC extract (≥100 μg/ml) and HC-HA/PTX3 (≥6.25 μg/ml) but not HA. Similarly, the metabolic activity in PC-3 cells was significantly inhibited by UC extract (≥200 μg/ml) and HC-HA/PTX3 (≥1.56 μg/ml) but not HA. Cell death was markedly noted with microscopy in both LNCaP and PC3 cells with HC-HA/PTX3 at concentrations ≥25 μg/ml.

Discussion

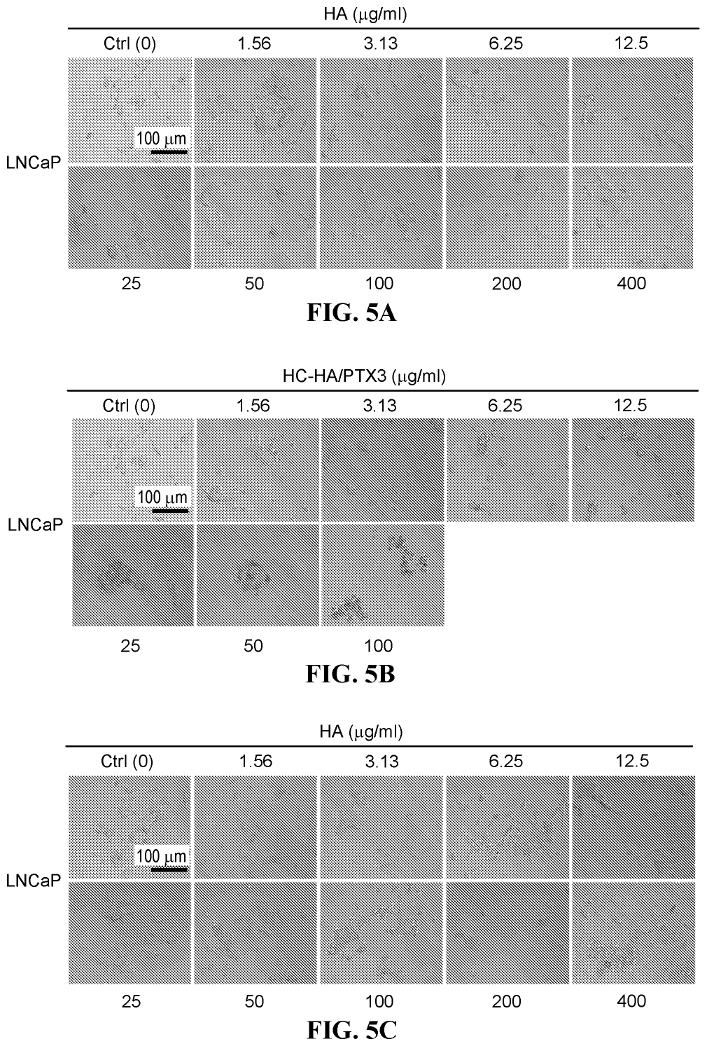

There was a clear inhibition of metabolic activity by HC-HA/PTX3 as determined by WST-1 and morphological evaluation. Based on morphology, PC3 and LNCaP cells both became rounded, less adherent to the matrix, and there were fewer cells when exposed to higher concentrations of HC-HA/PTX3. A difference was that the LNCaP cells aggregate as clusters with small diameter, rounded cells at higher HC-HA/PTX3 concentrations, however the PC3 cells did not aggregate. It remains unknown why LNCaP aggregated whereas PC3 did not when exposed to HC-HA/PTX3, however LNCaP cells have been shown to have faster aggregation rates than PC3. This may be due to their attachment dependence, with LNCaP and PC3 being anchorage-dependent and independent, respectively. For LNCaP, attachment to a surface is needed for viability and proliferation, and detachment induces cell death through the process of anoikis. The literature has compared the effect of LNCaP grown on different coating reagents (poly-1-lysine, poly-1-ornithine, collagen from human placenta type IV, fibronectin, and laminin) and showed laminin and collagen type IV promoted cell aggregation (FIG. 7, taken at 24 hours). This aggregation is similar to the morphology seen in the experiments provided herein and may suggest HC-HA/PTX3 reduces the LNCaP cell-surface attachment. It is well known that different substrate characteristics, including surface charge, topography, hydrophobicity/hydrophilicity, surface chemistry and surface energy may influence cell behavior and the modified cell-substrate interaction could affect the generation of intracellular signals. In fact, the liquid overlay technique is commonly used in this field to induce aggregation/spheroids by culturing cells on surfaces with non-adherent properties and thus cell-cell interactions are more prominent than those established between the cells and the surface. Consequently, cells aggregate leading to the formation of spheroids in 1-3 days, for the majority of the cell lines.

Conclusion

WST-1 assay data showed the metabolic activity of LNCaP cells was significantly inhibited (p<0.05) when treated with UC extract (≥100 μg/ml) and HC-HA/PTX3 (≥6.25 μg/ml) but not HA. Similarly, the metabolic activity in PC-3 cells was significantly inhibited by UC extract (≥200 μg/ml) and HC-HA/PTX3 (≥1.56 μg/ml) but not HA. Cell death was markedly noted with microscopy in both LNCaP and PC3 cells with HC-HA/PTX3 at concentrations ≥25 μg/ml.

These data showed the inhibition of metabolic activity in prostate cells by UC extract was independent of salt effects. Further, it was demonstrated here that AM and UC products will not promote cancer recurrence after prostatectomy.

### Example 3: Determine the Effect of HC-HA/PTX3 and HA on Morphology and Metabolic Activity of Normal Prostate Cells/Cell-Lines and Compare with Prostate Cancer Cell-Lines

The prostate epithelium is composed of two histologically distinct layers: the secretory luminal layer and basal cell layer. Human normal prostate epithelial basal and luminal cells were used. Human normal prostate epithelial basal cells (PrEC) were obtained from Clonetics-BioWhittaker, Inc. (Walkersville, Md., USA), and were cultured in prostate epithelial basal medium with PrEgM BulletKit (both from Clonetics) containing supplements and growth factors (BPE, hydrocortisone, hEGF, epinephrine, insulin, triiodothyronine, transferrin, gentamicin/amphotericin B, and retinoic acid).

Human normal prostate luminal PNT2 Cell Line was purchased from Sigma (cat 95012613). The cell line was established by immortalization of normal adult prostatic epithelial cells by transfection with a plasmid containing SV40 genome with a defective replication origin. The PNT2 cells were cultured in in RPMI 1640 medium supplemented with 10% fetal bovine serum (FBS), L-glutamine (2 mM), penicillin (100 U/ml), and streptomycin (100 μg/ml) (RPMI 1640 complete medium) at 37° C. in a humidified incubator in an atmosphere of 5% CO2 in air. Upon 70-80% confluency, cells were harvested by 0.25% (w/v) Trypsin-0.53 mM EDTA solution and were stored in 95% complete medium and 5% DMSO at liquid nitrogen vapor phase in aliquots.

TABLE 1
Cell metabolism analysis by WST-1 assay:

| | |
|---|---|
| | No. of |
| Sample | wells |

Unstimulated cells

| | |
|---|---|
| No treatment | 3 |
| No treatment + WST-1 solution [WST-1 control] | 3 |

Stimulated cells

| | | |
|---|---|---|
| HC-HA/PTX3 | 1.56 μg/ml for 48 h | 3 |
| | 3.13 μg/ml for 48 h | 3 |
| | 6.25 μg/ml for 48 h | 3 |
| | 12.5 μg/ml for 48 h | 3 |
| | 25 μg/ml for 48 h | 3 |
| | 50 μg/ml for 48 h | 3 |
| | 100 μg/ml for 48 h | 3 |
| HA | 1.56 μg/ml for 48 h | 3 |
| | 3.13 μg/ml for 48 h | 3 |
| | 6.25 μg/ml for 48 h | 3 |
| | 12.5 μg/ml for 48 h | 3 |
| | 25 μg/ml for 48 h | 3 |
| | 50 μg/ml for 48 h | 3 |
| | 100 μg/ml for 48 h | 3 |

Cells were revived in their respective growth medium. Cells were then plated at 3.2×10⁴/ml in 96-well plate (i.e. 3200 cells+100 ul/well) (n=3 for each assay) overnight for WST-1 assay. Purified HC-HA/PTX3 [AM2P (F 3-9) [TGAM17H008]; stock amount=67 μg] and HMW-HA (Healon; Cat #CE0344; lot #UP30583; stock concentration=10 mg/ml) were serially diluted with respective culture medium to get the aforementioned concentrations. Cells were treated with a series of doses of purified HC-HA/PTX3 or HA and were kept at 37° C. for 48 h. The cell morphology was recorded under microscope (bright-field in 10× and 20× magnifications) before treatment and after 24 h and 48 h of treatment. The metabolic activity of each cell-line was measured by WST-1 (cat #10008883, Cayman Chemical Company, Ann Arbor, Mich.) following the manufacturer's instructions (OD450 or OD450-OD670) at 48 h.

### A. Amplification of Normal Human Prostate Cells/Cell Lines

Human normal prostate epithelial basal cells (PrEC) grow faster than human normal prostate luminal cells (PNT2). For PrEC doubling time is 18-24 h, while for PNT2, it is longer about 36-48 h.

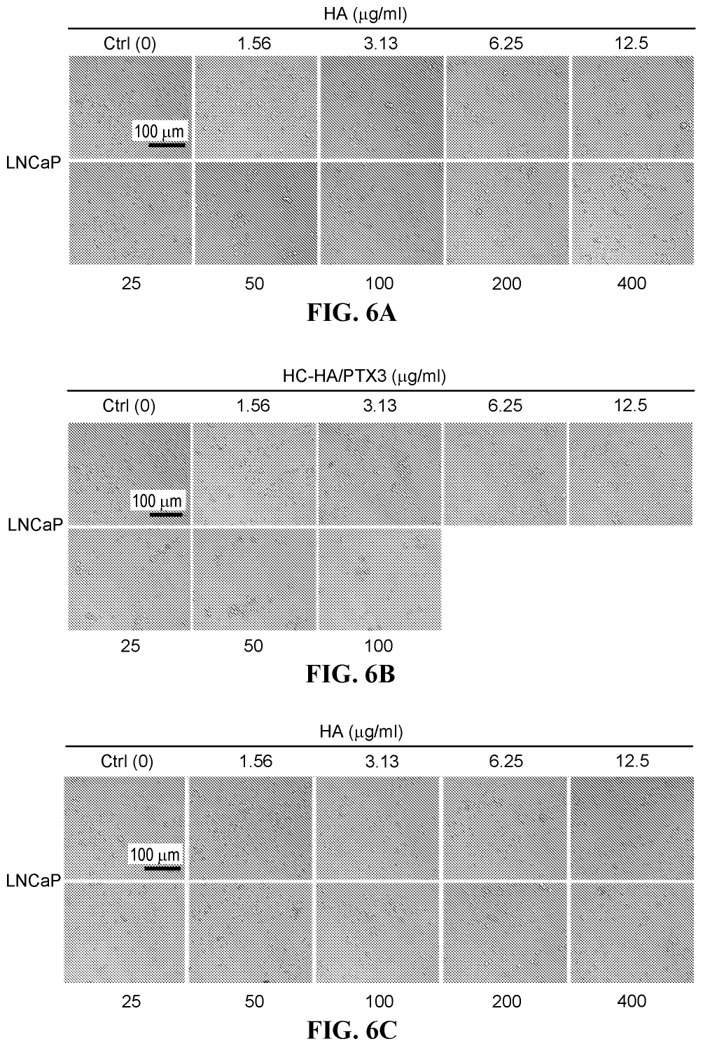

Bright-field images of PrEC (FIG. 8A) and PNT2 (FIG. 8B) prostate cell lines taken in 10× and 20× magnifications show morphology of normal prostate cells.

### B. Effect of HC-HA/PTX3 and HMW-HA on Human Normal Prostate Cell Morphology

PrEC cells typically proliferated fast and were well attached on the surface. Usually cells were well adhered to each other.

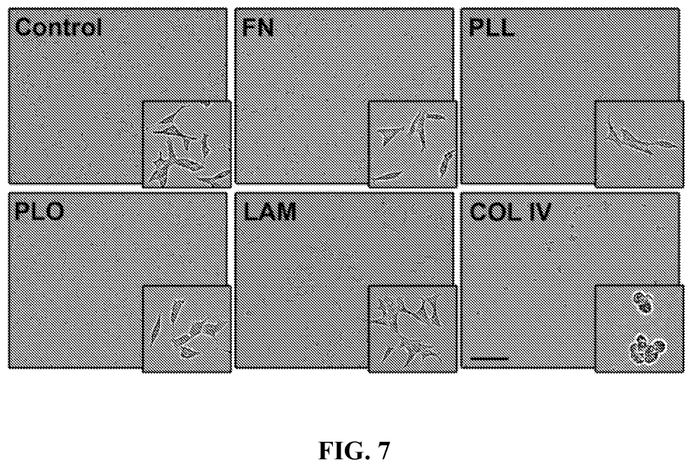

Following 24 hours HC-HA/PTX3 treatment: at 6.25 μg/ml, cells lost inter-cellular adhesion; at 12.5 μg/ml, loss of intercellular adhesion was increased; at 25 μg/ml, cells gradually started changing their morphology; at 50 μg/ml, cells gradually turned round and lost cell-cell adhesion; at 100 μg/ml, cells became round and diminished in sized, however, they were not dead. Following 48 hours HC-HA/PTX3 treatment: at 3.13 μg/ml, cells started dissociating; at 25 μg/ml, cells were completely dissociated from each other and did not grow in aggregate; at 50 μg/ml, cells were stressed, diminished in size, and became smaller and round; at 100 μg/ml cells died off and cell counts fell drastically (FIG. 9).

No significant effect of HA treatment was observed.

### C. Effect of HC-HA/PTX3 and HA on Human Normal Prostate Cell Metabolism

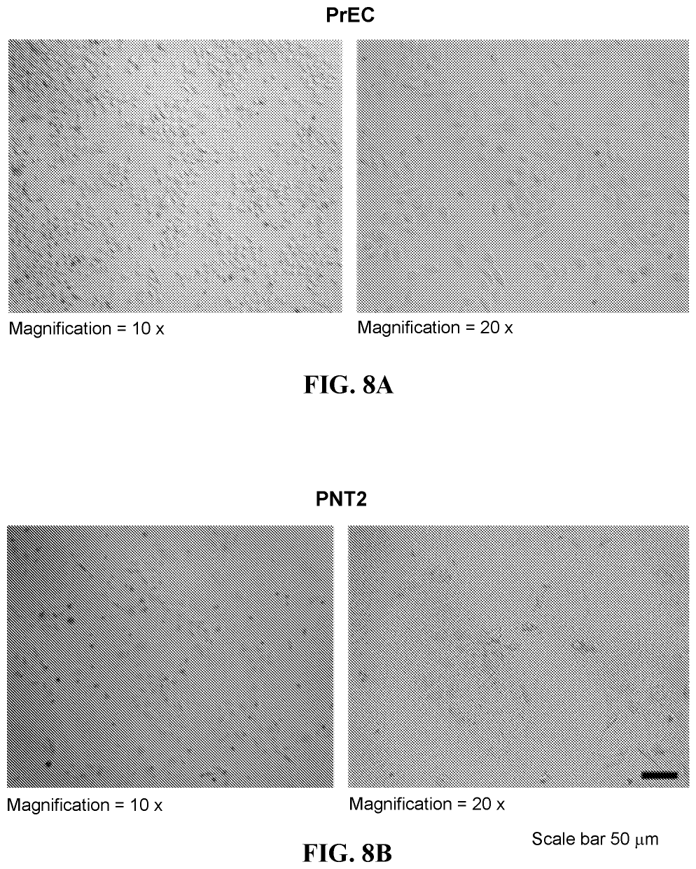

Metabolic activity of PrEC and PNT2 decreased significantly in a dose-dependent manner following treatment with HC-HA/PTX3. In contrast, HA did not have a significant effect on cell metabolism (FIG. 10A and 10B). Between these two types of normal prostate cells, PrEC (FIG. 10A) was more sensitive to very low concentrations of HC-HA/PTX3 (1.56 and 3.125 μg/ml). The same low concentration of HC-HA/PTX3 (1.56 and 3.13 μg/ml) did not affect PNT2 (FIG. 10B) cell metabolic activity.

Metabolic activity (%) was evaluated in the normal human primary prostate epithelial cells PrEC and normal human prostate cell line PNT2 by WST-1 assay after 48 hr. incubation with different concentrations (0.78, 3.125, 6.25, 12.5, 25, 50, 100 μg/ml) of HC-HA/PTX3 or HA. P-value calculated by 2-tailed t-test with respect to the untreated samples.

D. Comparative Analysis of the Cell Metabolic Activity Among Normal & Cancer Prostate Cells/Cell Lines Under Treatment with HC-HA/PTX3 and HA

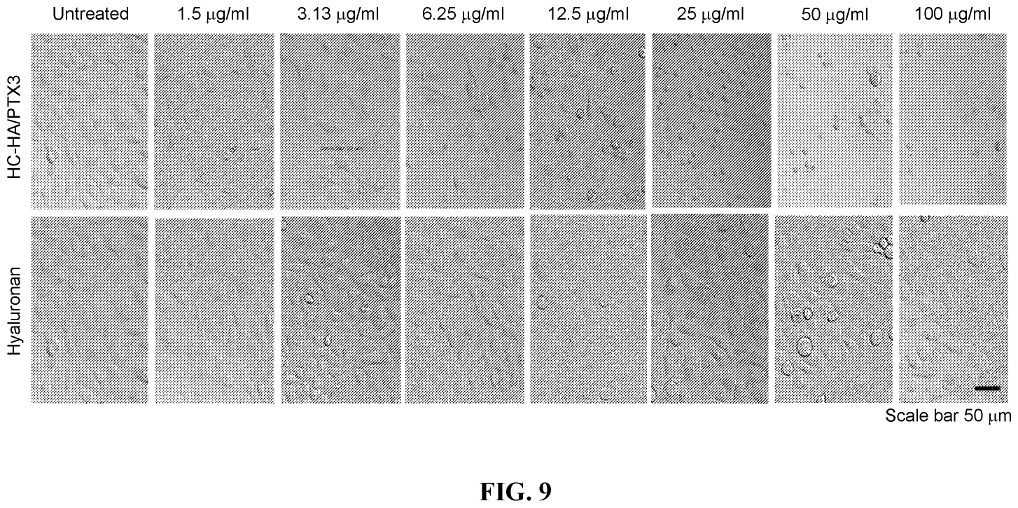

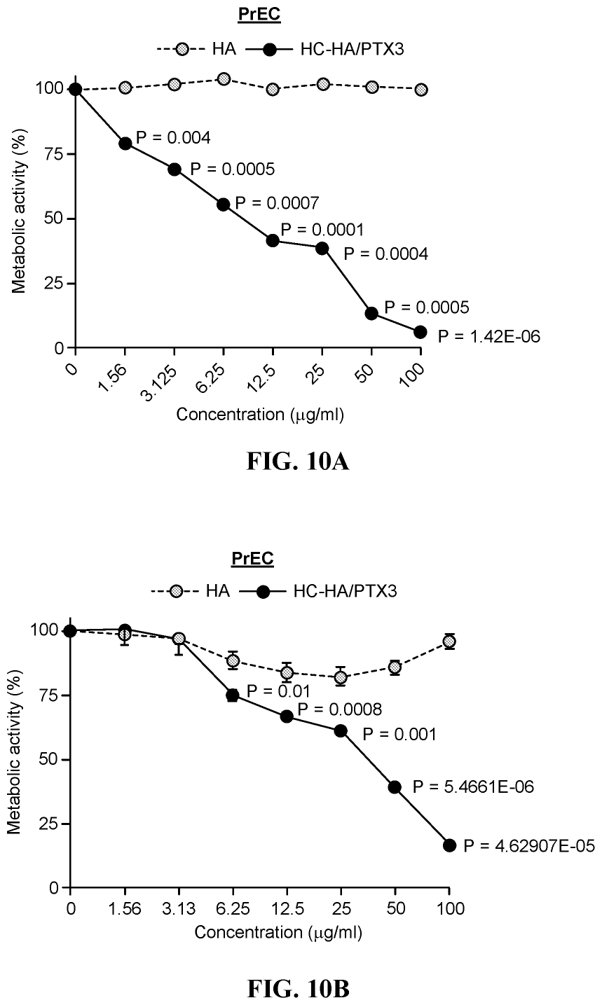

The effect of different concentrations of HC-HA/PTX3 (FIG. 11A and 11B) and HA (FIG. 12A and 12B) on the metabolic activity of both normal prostate cells (PrEC & PNT2) and prostate cancer cell lines (LNCaP & PC3) was compared. Both normal and cancer cell types reacted to HC-HA/PTX3 and HMW-HA in a similar manner. At 25 μg/ml HC-HA/PTX3, both PrEC cells and PNT2 cell line showed less sensitivity compared to the cancer cell lines (LNCaP and PC3). There was not a significant difference in metabolic activity of PrEC, PNT2, PC3, and LNCaP cells following treatment with HMW-HA.

FIGS. 11A-11B and FIGS. 12A-12B show comparative analysis of the metabolic activity (%) evaluated in the normal primary prostate epithelial cells (PrEC) and cell lines (PNT2) and prostate cancer cell lines: PC3 and LNCaP by WST-1 assay after 48 hr incubation with different concentrations (0.78, 3.125, 6.25, 12.5, 25, 50, 100 μg/ml) of HC-HA/PTX3 and HA. Semi-logarithmic regression analysis demonstrated, HC-HA/PTX3 inhibited metabolic activity of all types of prostate cells linearly in a dose-dependent manner while HA did not have any significant effect * (p<0.05); ** (p<0.01); *** (p<0.001).

Overall, HC-HA/PTX3 inhibited cell metabolic activity of all types of human prostate cells/cell lines, including normal and cancer in dose dependent manner while HMW-HA had no significant effect. At concentrations up to 25 μg/ml PNT2 cell line showed less sensitivity towards HC-HA/PTX3 (cell metabolic activity: 60%; p<0.001) while the metabolic activity of the cancer cell lines (both PC3 and LNCaP) were reduced below 25% (p<0.001).

### Example 4: Determine the Effects of HC-HA/PTX3 and HA on the Morphology and Metabolic Activity of the Selected Cancer Epithelial Cell Lines

The above examples showed that HC-HA/PTX3 regulated metabolic activity of two prostate cancer cell-lines (PC-3 and LNCaP). In this study, the effect of HC-HA/PTX3 and HMW-HA (as control) on the morphology and metabolic activity of 4 different human cancer cell lines: A-375 (melanoma), A549 (lung cancer), MCF-7 (breast cancer) and HT-29 (colon adenocarcinoma) was tested by WST-1 assay. The concentrations of HC-HA/PTX3 and HA were selected based on the results shown in Example 1.

It was noted in Example 1, that the HC-HA/PTX3-response curve plateaus at concentrations of 50 μg/ml and above. There was no significant variation in metabolic activity between 50 μg/ml and 100 μg/ml concentration of HC-HA/PTX3. As such, the maximum concentration of HC-HA/PTX3 was maintained at 100 μg/ml.

Cells were thawed and resuspended in 5 ml pre-warmed fresh complete culture medium followed by centrifugation at 250 g for 5 min. The supernatant was carefully removed and the cell pellet was resuspended in 10 ml pre-warmed fresh complete culture medium and seeded in 100 mm culture plates and was kept at 37° C. After 5 days culture, A-549, HT-29 & A357 cells were almost 70-80% confluent, while MCF-7 cells grew slowly. Cell plates were taken out of incubator and medium was carefully removed followed by washing twice with 3-5 ml D-PBS. Cells from each 100 mm plate were then harvested using 5 ml 0.25% Trypsin-0.53 mM EDTA. After 5-10 min, 5 ml fresh culture medium was added to neutralize the effect of trypsin-EDTA and the cells were centrifuged at 250 g for 5 min. After centrifugation, supernatant was carefully removed, and the cell pellet was resuspended with 2 ml fresh culture medium. For each cell-line, 10 μl of cell suspension was mixed with 10 μl trypan blue to stain dead cells, and the viable cell number was counted by hemocytometer under microscope. In this study four 96-well plates, one for each cell line, were used. Cell number to be seeded was determined based on previous Example (seeding cell count: 1×10⁴/cm² in Example 1 and 2). Given the area of each well of a 96-well plate is 0.32 cm², 3.2×10⁴/ml cells were needed. Each well was seeded with 3200 cells in 100 μl culture medium. Cells were treated with the following concentrations of HC-HA/PTX3 and HA (as control): 0.78, 1.56, 3.125, 6.25, 12.5, 25, 50, 100 (μg/ml) in triplicate. Two untreated samples were used: untreated without WST-1 reagent and untreated with WST-1 reagent (as control for WST-1 assay). To avoid pipetting error, cells were diluted to a final volume of 8 ml in culture medium as shown in Table 2.

TABLE 2
Dilution scheme to yield 3200 cells in 100 μl culture medium

| | | | | Culture medium added |
|---|---|---|---|---|
| Cell lines | Cell count | Cells added | | to make total volume 8 ml. |
| MCF-7 | 25.2 × 10⁴/ml | 1 | ml | 7 ml |
| HT-29 | 261.6 × 10⁴/ml | 98 | μl | 7 ml 902 μl |
| A-549 | 105.8 × 10⁴/ml | 242 | μl | 7 ml 758 μl |
| A-375 | 160.6 × 10⁴/ml | 160 | μl | 7 ml 840 μl |

After incubation at 37° C. for overnight, cell morphology was captured under microscope at bright-field at magnification: 10× and 20×, respectively. Purified HC-HA/PTX3 [AM2P (F 3-9); purified on Feb. 2, 2018; stock amount=25.78 μg] and HMW-HA (Healon; Cat #CE0344; lot #UP30583; Exp. Date: December 2018; stock concentration=10 mg/ml) were serially diluted with respective culture medium to get the required concentrations (i.e., 0.78, 1.56, 3.125, 6.25, 12.5, 25, 50, 100 μg/ml). Cells were treated with a series of doses of purified HC-HA/PTX3 or HA and were kept at 37° C. incubator for 48 h. The cell morphology was recorded under microscope (bright-field in 10× and 20× magnifications) before treatment and after 24 h and 48 h of treatment. The metabolic activity of each cell-line was measured by WST-1 (cat #10008883, Cayman Chemical Company, Ann Arbor, Mich.) following the manufacturer's instructions (OD450 or OD450-OD670) at 48 h.

Effect of HC-HA/PTX3 or HMW-HA on human cancer epithelial cell morphology:

a. A-375 (melanoma): Normally, cells proliferate fast and are well attached on the surface.

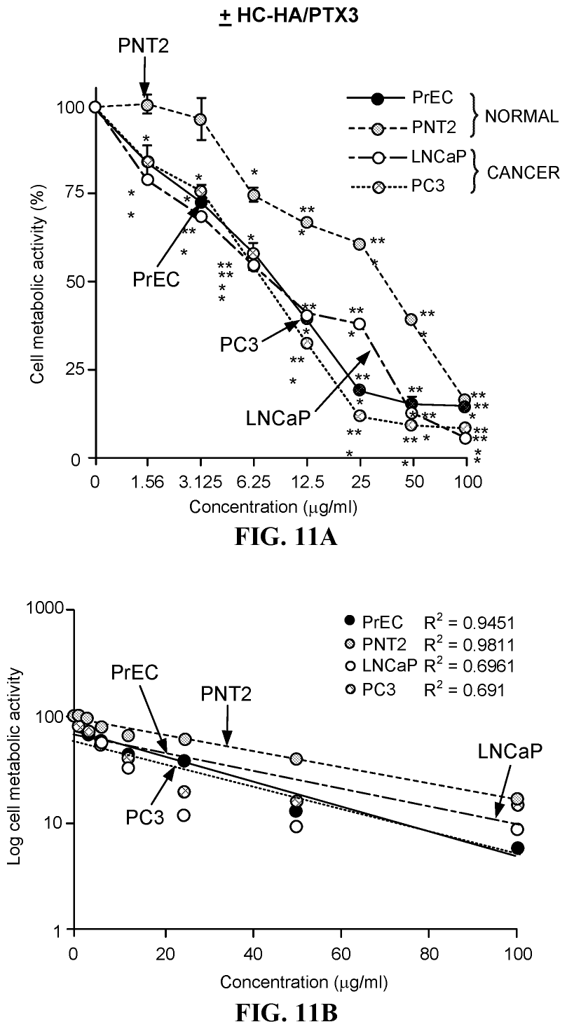

i. HC-HA/PTX3 treatment: After 24 h: at 25 (μg/ml): cells tended to be spindle shaped. At 50 (μg/ml): cells turned spindle shaped. At 100 (μg/ml): cells were round and dead. After 48 h: at 6.25 (μg/ml): cells showed slightly lose intercellular adherence. At 12.5 (μg/ml): Cells lost more intercellular adherence. Cells tended to grow individually rather than in clump. At 25 (μg/ml): >80% cells were shrinking, spindle shaped, remaining cells were round. At 50 (μg/ml): >60% cells were dead and appeared round, remaining cells were spindle-shaped. At 100 (μg/ml): all cells appeared as round and were dead. See, FIG. 13.

ii. HA treatment: No significant effect of HA observed.

iii. Results showed that as the concentration of HC-HA/PTX3 increased, A-375 cells broadly changed morphology in a dose-dependent manner according to the following steps:

Normal epithelial cells adhered to each other>Cells lose inter-cellular attachment and turn spindle shaped. Cell shrinkage>Cells turn round and die.

a. HT-29 (colon cancer): cells are normally round and grow in aggregate. Each aggregate appears like a ball.

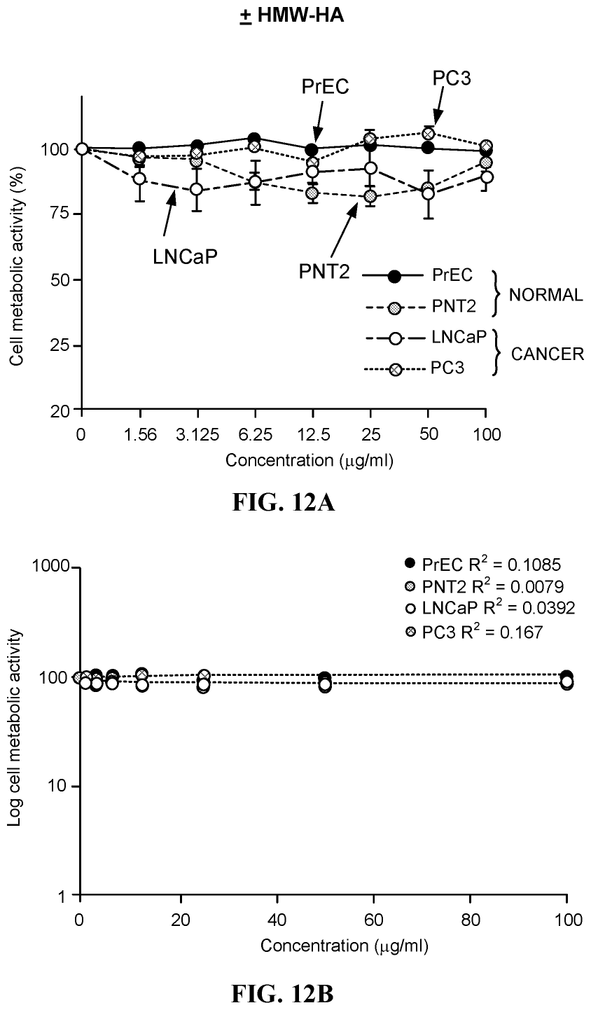

i. HC-HA/PTX3 treatment: After 24 h: at 50 (μg/ml): cells lost adhesion and were isolated. At 100 (μg/ml): cells appeared as round bead-like shape and survived less. After 48 h: at 1.56 (μg/ml): intercellular adherence was gradually lost. At 6.25 (μg/ml): <10 cells adhered to each other in each clump. Cells lost intercellular adherence more. At 25 (μg/ml): cells tended to grow in single rather than in aggregate. At 50 (μg/ml): cells grew in single. At 100 (μg/ml): single cells round shaped and survived less. See, FIG. 14.

ii. HA treatment: No significant effect of HA observed.

iii. As the concentration of HC-HA/PTX3 increased in a dose-dependent manner, broadly HT-29 cells changed morphology in the following steps:

>20 cells adhered to each other. Appears like a grape. >Intercellular adherence gradually loosens><10 cells adhere to each other in each clump>cells gradually come out of the clump. Intercellular and cell-matrix adhesiveness decreases, rather than in aggregate.>Single round cells survive less.

a. A549 (lung cancer): Normally cells are epithelial and proliferate quickly.

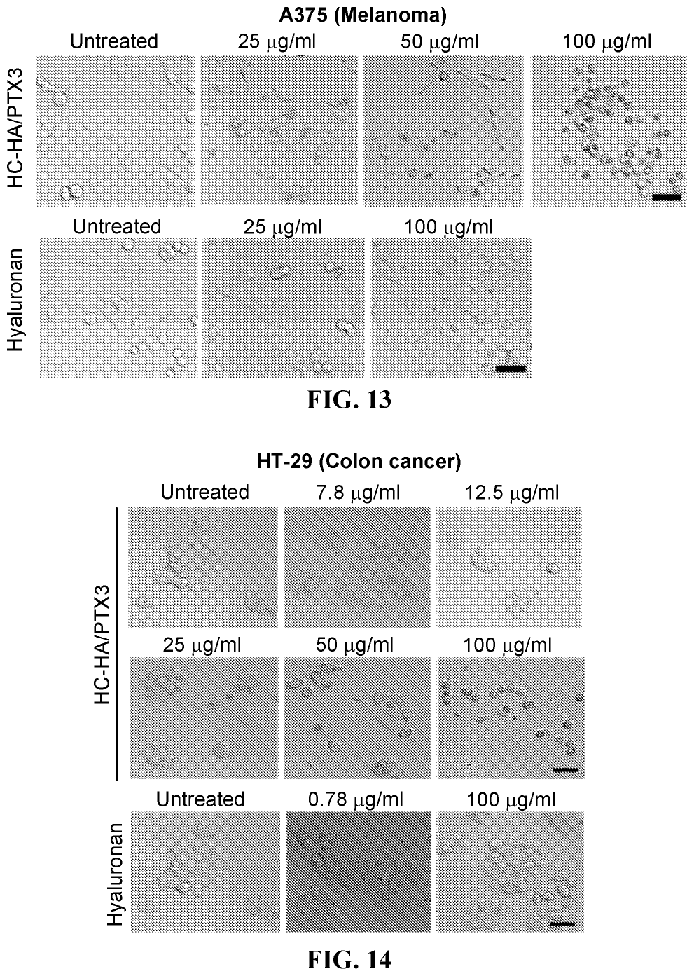

i. HC-HA/PTX3 treatment: After 24 h: at 25 (μg/ml): cells morphology slightly changed to spindle shaped. At 50 (μg/ml) cells tend to be more spindle shape. At 100 (μg/ml): All cells are not dead. Dead cells are round. After 48 h: at 12.5 (μg/ml): cell morphology slightly changed to spindle shape. At 50 (μg/ml): cells tend to be more spindle shape. At 100 (μg/ml): All cells are not dead. Dead cells are round. See, FIG. 15.

ii. HA treatment: No significant effect of HA observed.

iii. Under HC-HA/PTX3 treatment, broadly A-549 cells changed their morphology in the following steps:

Cells proliferate. Intercellular adherence. >Cells round & dot-like. Dead

a. MCF-7 (breast cancer): Normally cells are epithelial. Slow growing. Adhered to each other. Grow in aggregate.

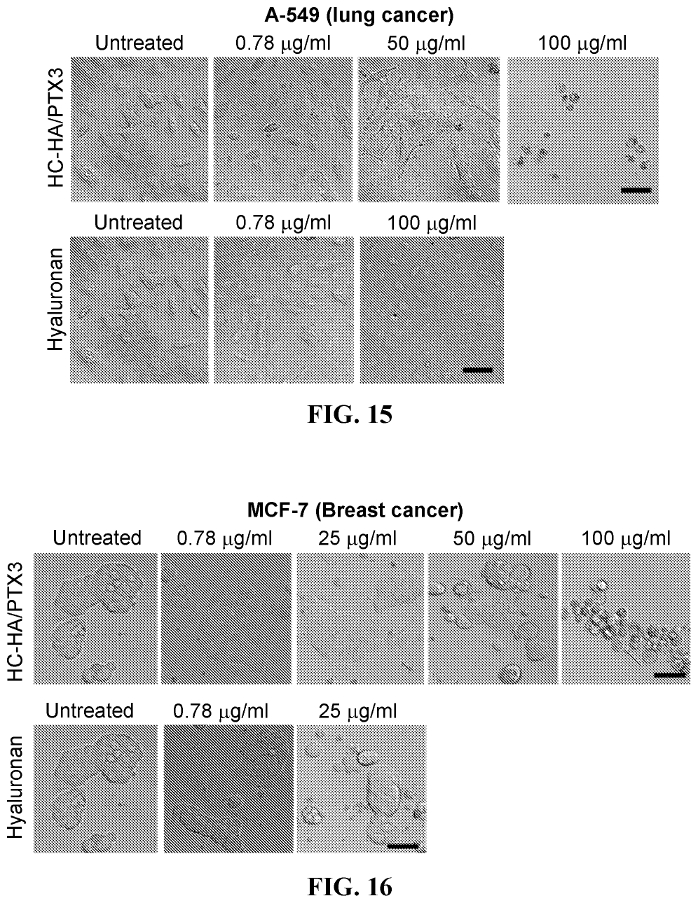

i. HC-HA/PTX3 treatment: After 24 h: No effect until 50 μg/ml. At 100 (μg/ml): Round bead like cells dead. After 48 h, the effect was same. See, FIG. 16.

ii. HA treatment: No significant effect of HA observed.

iii. Under HC-HA/PTX3 treatment, broadly MCF-7 cells changed their morphology in the following steps:

Cells grow in aggregate>Cells dissociated. Dead.

Effect of HC-HA/PTX3 or HMW-HA on Metabolic Activity of the Cancer Cell-Lines:

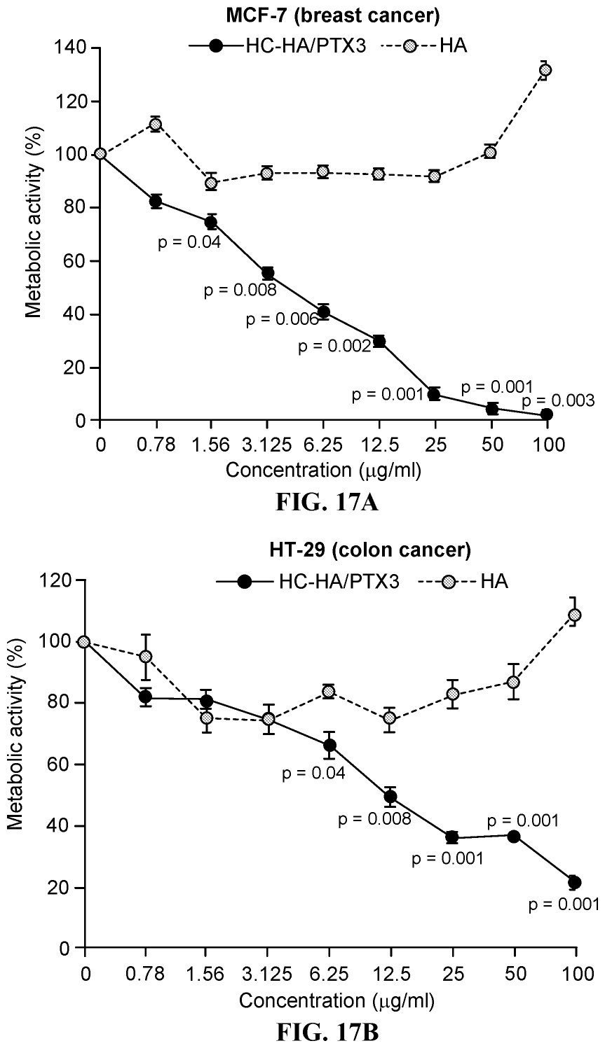

A375: A-375 cell metabolism decreased significantly in a dose-dependent manner. After 48 h treatment with HC-HA/PTX3, cell metabolism changed significantly from 1.56 μg/ml onwards. No significant effect of HA was observed. (FIG. 17A)

HT-29: HT-29 cell metabolism decreased significantly in a dose-dependent manner. After 48 h treatment with HC-HA/PTX3, cell metabolic activity changed significantly from 6.25 μg/ml onwards. No significant effect of HA was observed. (FIG. 17B)

A549: A549 cell metabolism decreased significantly in a dose-dependent manner. After 48 h treatment with HC-HA/PTX3, cell metabolic activity changed from 0.78 μg/ml onwards. No significant effect of HA was observed. (FIG. 17D)

MCF-7: MCF-7 showed the least sensitivity to HC-HA/PTX3. Significant effect of HC-HA/PTX3 could be observed at a high concentration (50 μg/ml) but the effect was not as strong as observed for other cell lines like A375 and HT-29. (FIG. 17C)

HC-HA/PTX3 inhibited cellular aggregation, intercellular junction, cell shape and cell adhesion, thus cell metabolic activity of all the 4 types of cancer cells tested in a dose-dependent manner. In contrast, Hyaluronan had no significant effect on the cellular morphology and metabolic activity of all four cell types. These effects occurred rather soon, e.g., in 24 h, strongly suggesting that HC-HA/PTX3, upon binding with CD44, results in a change in cytoskeletons/membrane interaction, which is specific and different from HMW HA.

Out of the 4 cell-lines, A375 and HT-29 were more sensitive to HC-HA/PTX3 than A549 and MCF-7. Because MCF-7 does not express CD44 significantly, the difference among these four tumor cells might be linked to the degree of CD44 expression.

The change in intercellular and substrate adhesion is different between A375 and HT-29 upon contact with HC-HA/PTX3. For A375, a morphological change was seen after detachment: cells first turn spindle-shaped and then become round. In contrast, HT-29 did not show any morphological change. Cells remained round-shaped from the beginning. Under high concentration of HC-HA/PTX3, they gradually lost the intercellular attachment. These results suggest that in different types of cancer, HC-HA/PTX3 may regulate cell adhesion signaling by different pathways.

The major events observed in these cancer cell-lines were: inhibition of cell metabolic activity, leading to cell shape change, leading to cell death.

### Example 5: Determine the Effect of HC-HA/PTX3 and HA on Mesenchymal Cells

In examples 2 and 4, it was identified that HC-HA/PTX3 affects the morphology and metabolic activity of normal prostate epithelial cells similar to those of tumor cells. To address if such an effect also applies to normal mesenchymal cells, it was examined whether HC-HA/PTX3 also exerts similar effects on the morphology and the metabolic activity of a series of mesenchymal cells.

Three normal mesenchymal primary cells were used: (a) limbal niche cells (LNC), (b) human trabecular meshwork (HTM), and (c) human corneal fibroblast (HCF) respectively. Cells were cultured in their respective growth medium. LNC was cultured on 5% matrigel (without serum) in HSCM medium. HTM cells were cultured on 5% matrigel with 5% FBS in ESCM medium. HCF cells were cultured only on plastic with 10% FBS in DMEM. Cells were cultured on each well of a 6-well plate. For LNC and HTM cells, wells were first coated with 1000 μl 5% matrigel and incubated at 37° C. for 1 h. Subsequently, cells were seeded in each well in 1000 μl culture medium. After cells achieved sufficient confluency, they were trypsinized followed by brief centrifugation at 200 g for 5 mins. Medium was removed and the cell pellet was supplemented with fresh culture medium. Selected wells of a 96-well plate were coated with 50 μl 5% matrigel and incubated at 37° C. for 1 hr. Afterwards, LNC and HTM cells were seeded in matrigel-coated 96-well plates and kept at 37° C. for overnight incubation. In each well, 3200 cells were seeded in 100 μl culture medium. On the following day, cells were treated with a series of doses (defined by HA concentration, e.g., 0, 1.56, 3.125, 6.25, 12.5, 25, 50, 100 μg/ml, n=3) of purified HC-HA/PTX3 and HA. The concentration of HC-HA/PTX3 and HA was calculated by serial dilution as done in Example 3. The cell morphology was recorded after 15-30 mins, 1 h, 5 h, 24 h and 48 h of treatments respectively. After 48 h incubation, the cell metabolic activity was measured by WST-1 (cat #10008883, Cayman Chemical Company, Ann Arbor, Mich.) according to the manufacturer's instructions (OD450 or OD450-OD670).

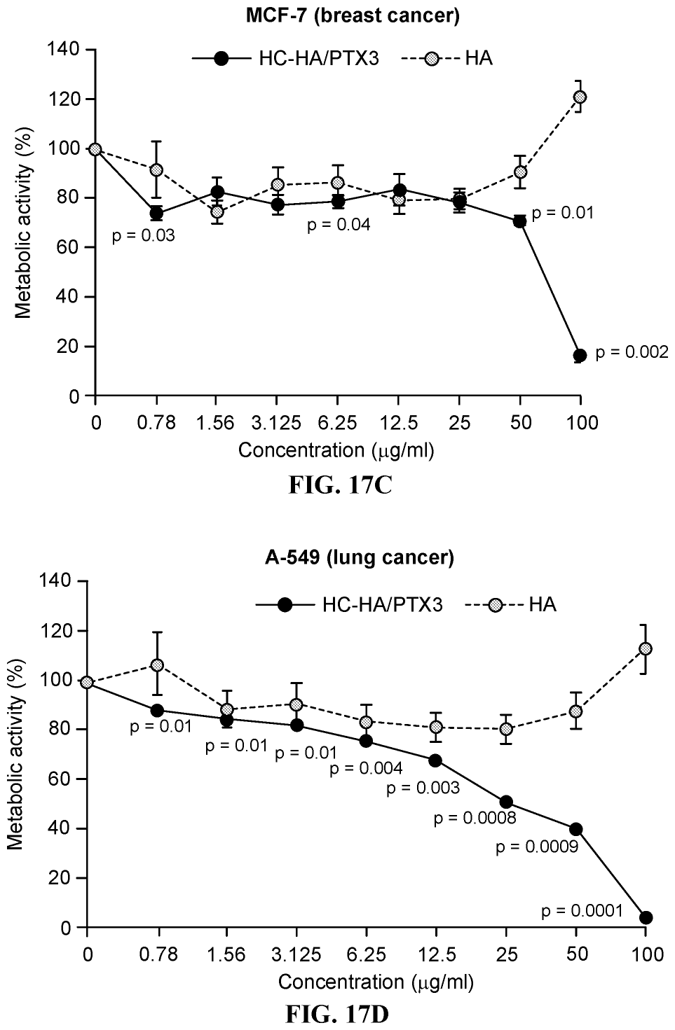

A change in Limbal Niche cell (LNC) morphology was observed within 15 mins with 25 to 100 μg/ml HC-HA/PTX3. However, after longer incubation (1 h) with HC-HA/PTX3 the cells reverted to the original shape again. The cell morphology did not change further (See, FIG. 18A).

Cells treated with high dose of HMW-HA (100 μg/ml) shrank more and became thread-like (FIG. 18B). LNC became flattened but comparatively shorter when treated with 100 μg/ml HC-HA/PTX3 for 48 h. FIG. 18C shows representative bright-field microscopic image (scale bar 50 μm) of LNC (limbal niche cells) after 48 h incubation with 100 μg/ml of HC-HA/PTX3 or HMW-HA.

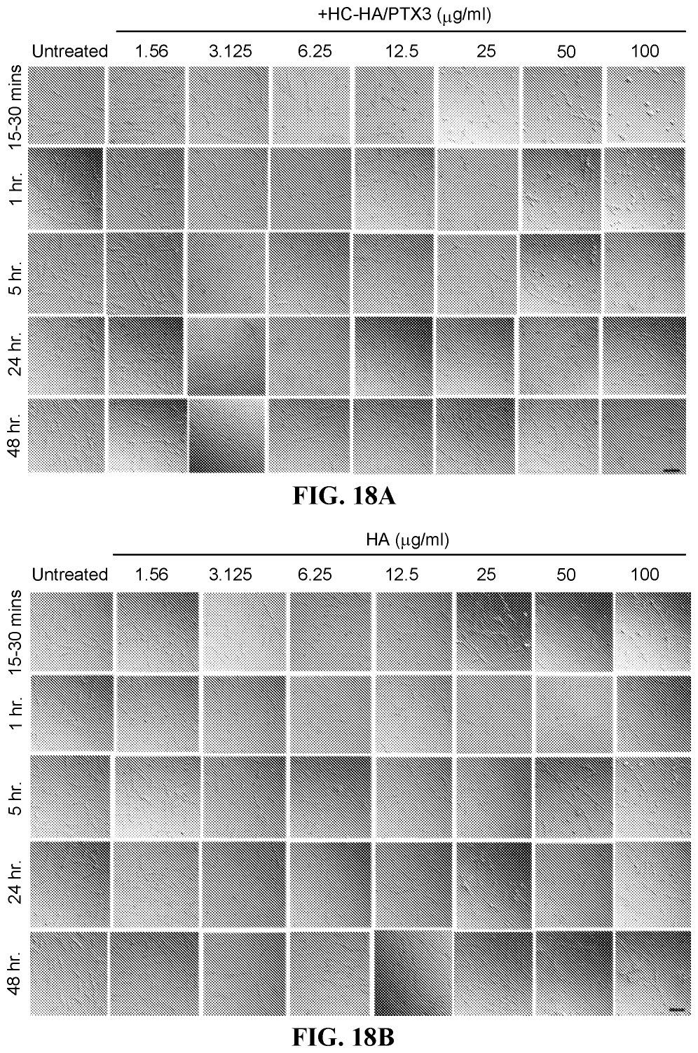

LNC were metabolically quite resistant to HC-HA/PTX3. After 48 h incubation, overall cell metabolic activity remained around 75%, implicating little effect of HC-HA/PTX3 on LNC, although with gradual increase in HC-HA/PTX3 concentration, cell metabolic activity decreases rapidly. Significant effect of HC-HA/PTX3 began at concentration 12.5 μg/ml. No significant change in metabolic activity of LNC was detected while treated with HMW-HA. FIG. 19 shows metabolic activity (%) evaluated in limbal niche cells by WST-1 assay after 48 h incubation with different concentrations (1.56, 3.125, 6.25, 12.5, 25, 50, 100 μg/ml) of HC-HA/PTX3 and HA. p-value calculated by 2-tailed Student's t-test with respect to the untreated samples.

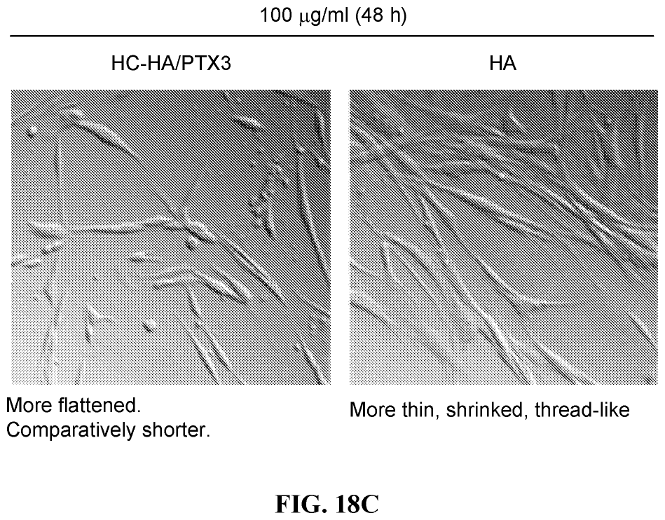

Human trabecular meshwork (HTM) cells were more resistant to HC-HA/PTX3 compared to LNC. No morphological change within a short span of time was observed in HTM cells. The cell shape did not change during 48 h incubation with different concentrations of HC-HA/PTX3.5% FBS in the culture medium may make the cells more resistant to HC-HA/PTX3. Cell morphology did not change while treated with HMW-HA. FIG. 20A and 20B show representative bright-field microscopic images (scale bar 50 μm) of HTM (human trabecular meshwork) cells after treatment with different concentrations of HC-HA/PTX3 (FIG. 20A) and HMW-HA (FIG. 20B) for different time points: 15-30 mins, 1 hr, 5 hr, 24 hr and 48 hr. respectively.

HTM cells were very resistant to HC-HA/PTX3 as observed in cell morphology (FIG. 20A). After 48 hr. incubation, overall cell metabolic activity remained more than 75%, implicating little effect of HC-HA/PTX3 on HTM, although significant decrease in cell metabolic activity (p=0.04) was evident with highest concentration of HC-HA/PTX3 (100 μg/ml). No significant change in metabolic activity of HTM while treated with HMW-HA was detected (FIG. 20B).

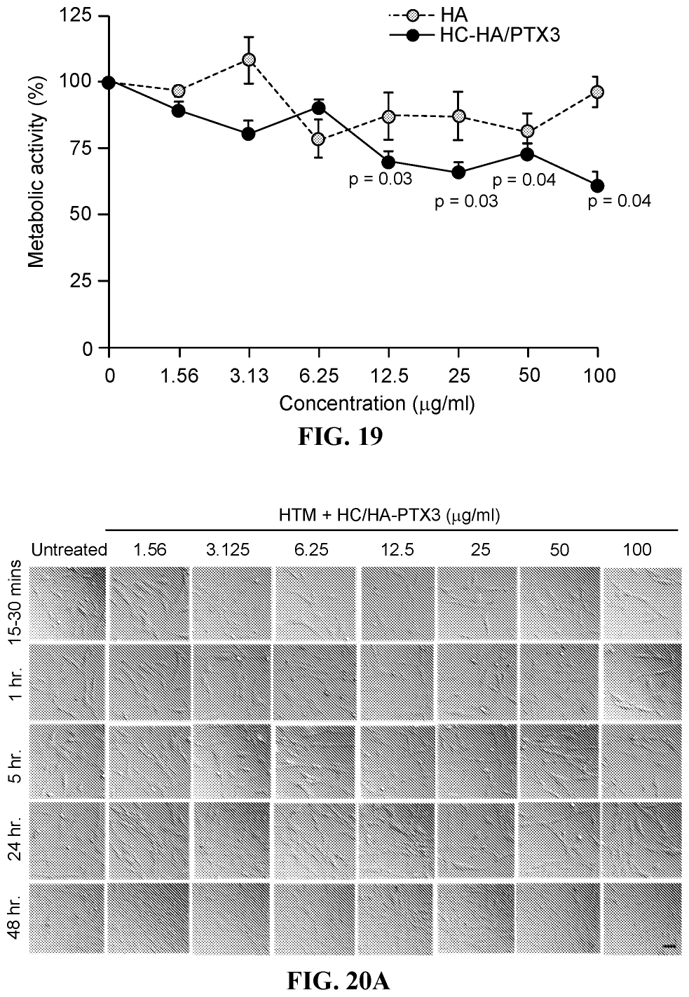

FIG. 21 shows metabolic activity (%) evaluated in human trabecular meshwork cells by WST-1 assay after 48 hr. incubation with different concentrations (1.56, 3.125, 6.25, 12.5, 25, 50, 100 μg/ml) of HC-HA/PTX3 and HA. p-value calculated by 2-tailed Student's t-test with respect to the untreated samples. As shown, HC-HA/PTX3 and HMW-HA had little effect on HTM.

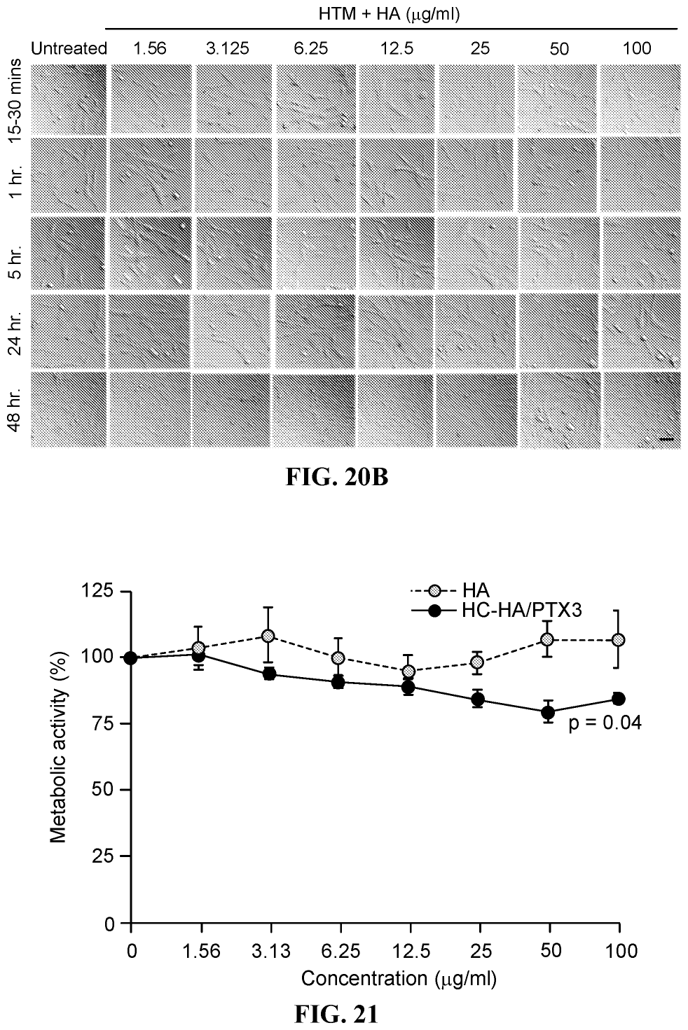

The effect of HC-HA/PTX3 on the morphology of human corneal fibroblasts (HCF) was almost similar to that on LNC. Within 15-30 minutes after treatment with 100 μg/ml of HC-HA/PTX3 cells became round, but they recovered back to their original morphology by an hour. Afterwards cell shape did not change anymore even at longer incubation (48 h) with high concentration of HC-HA/PTX3. HMW-HA had no significant effect on HCF cell morphology. FIG. 22A and 22B shows representative brightfield microscopic images (scale bar 50 μm) of human corneal fibroblast (HCF) cells after treated with different concentrations of HC-HA/PTX3 (FIG. 22A) and HMW-HA (FIG. 22B) for different time points: 15-30 mins, 1 hr, 5 hr, 24 hr and 48 hr. respectively.

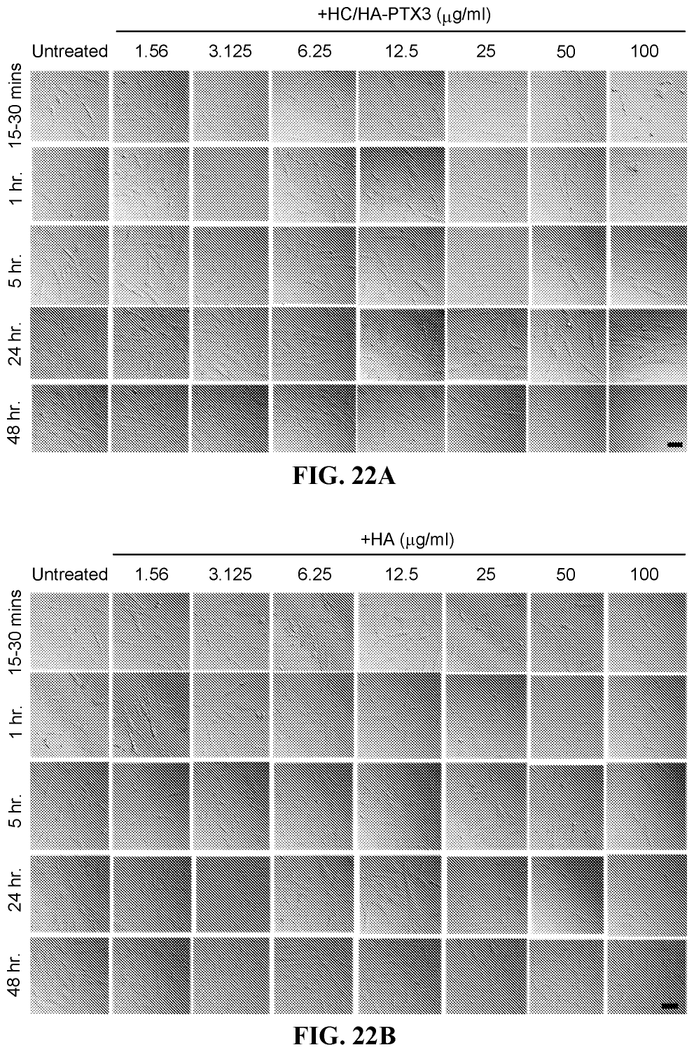

HC-HA/PTX3 inhibited cell metabolic activity of HCF cells in a dose-dependent manner while HMW-HA had no significant effect. Significant inhibition was observed at concentration 12.5 μg/ml onwards. FIG. 23 shows metabolic activity (%) evaluated in human corneal fibroblast cells by WST-1 assay after 48 hr. incubation with different concentrations (1.56, 3.125, 6.25, 12.5, 25, 50, 100 μg/ml) of HC-HA/PTX3 and HA. p-value calculated by 2-tailed Student's t-test with respect to the untreated samples.

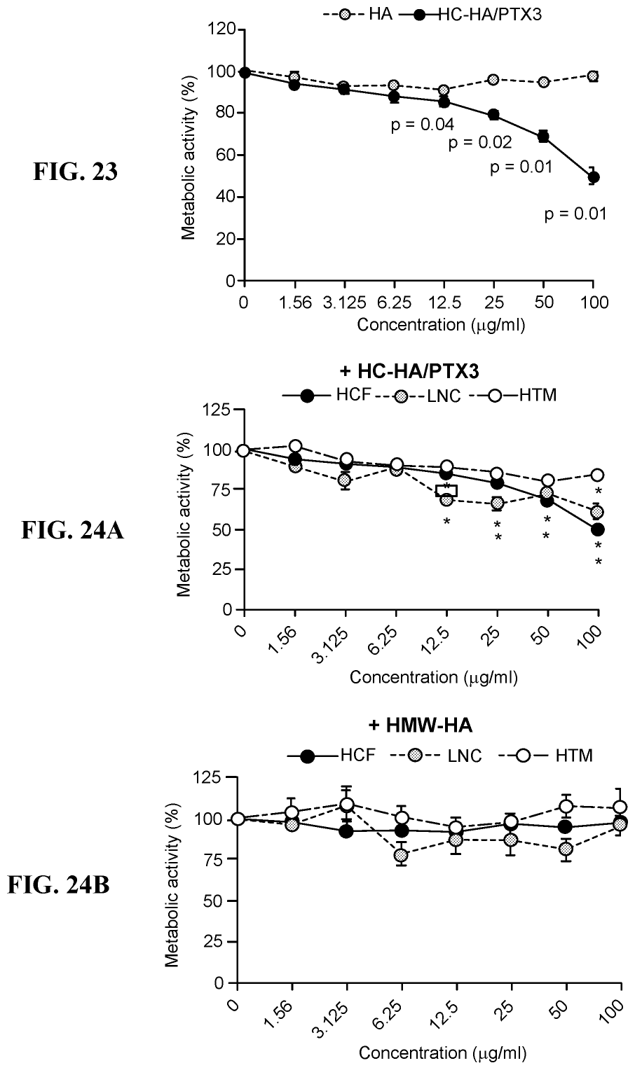

Overall, the human normal mesenchymal primary cells showed more resistance towards HC-HA/PTX3 compared to the epithelial cells (normal and cancer) (Table 3) albeit the cell metabolic activity was significantly inhibited by HC-HA/PTX3 in a dose-dependent manner. HMW-HA had no striking effect on cell morphology and metabolic activity of primary mesenchymal cells. Out of the three types of mesenchymal cells, HTM was more rigid towards high concentration (100 μg/ml) of HC-HA/PTX3 (metabolic activity: 84.8%; p=0.04) while the same concentration of HC-HA/PTX3 could suppress metabolic activity of HCF (50%; p=0.01) and LNC (61.4%; p=0.04) more. FIGS. 24A and 24B show comparative analysis of the metabolic activity (%) evaluated in three types of human normal primary mesenchymal cells: HCF, HTM & LNC as evaluated by WST-1 assay after 48 hr. incubation with different concentrations (1.56, 3.125, 6.25, 12.5, 25, 50, 100 μg/ml) of HC-HA/PTX3 (FIG. 24A) and HA (FIG. 24B). p-value calculated by 2-tailed Student's t-test with respect to the untreated samples. *denotes p<0.05.

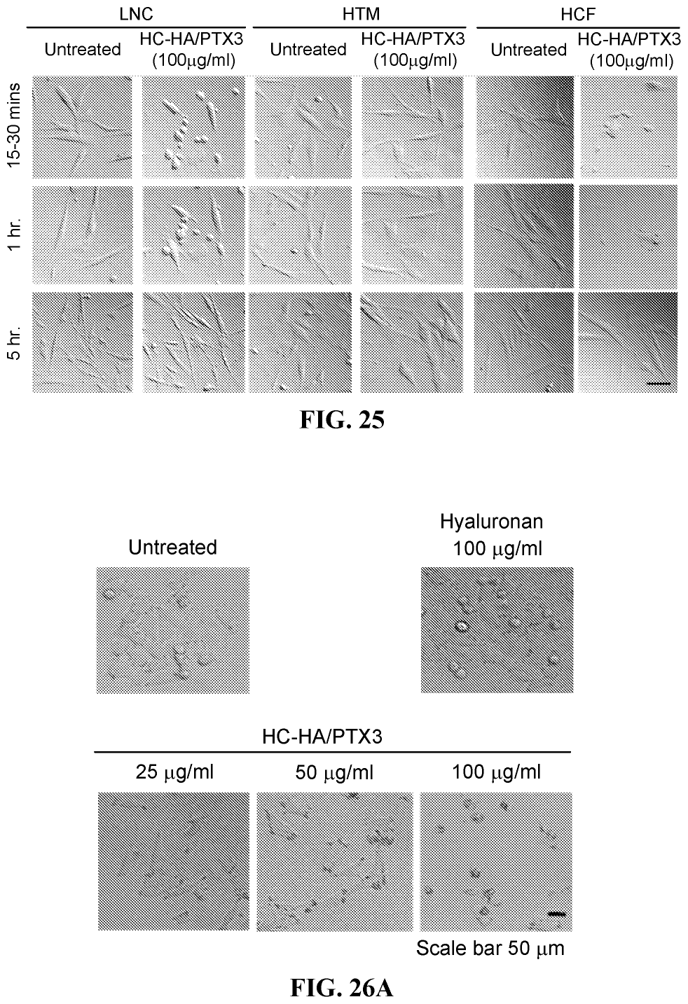

LNC and HCF were more sensitive to HC-HA/PTX3, the cell shape changed with high dose of HC-HA/PTX3 very quickly (within 15-30 mins), although it did not persist longer. Within 1 h, the cells recovered back to their normal shape, and afterwards cell morphology was not affected any more even with long incubation (48 h) with high concentration of HC-HA/PTX3. On contrary, HC-HA/PTX3 could not modulate HTM cell shape at high concentration both for shorter as well as longer incubation period. Representative bright-field microscopic images (scale bar 50 μm) (FIG. 25) show morphological aberration and quick recovery of mesenchymal cells (LNC & HCF) while challenged with HC-HA/PTX3 (100 μg/ml) within short span of time. HTM cells do not react to HC-HA/PTX3 like the other normal mesenchymal cells.

TABLE 3
Difference between mesenchymal and epithelial cells while treated
with high concentration of HC-HA/PTX3 (e.g., 100 μg/ml) for 48 h.

| Characteristic | Mesenchymal cells | Epithelial cells |
|---|---|---|
| Time of effect | Very quick | Slow, takes long time |
| Cell shape | Any changes occur | Cell shape change |
| | within the first 15 | occurs later and is |
| | mins. and revert to | persistent. |
| | original morphology | |
| | within one hour. | |
| Cell survival rate | High | low |
| Overall cell metabolic | High | low |
| activity | | |
| Cell proliferation rate | High | low |

### Example 6: Determine the Role of HC-HA/PTX3 in Suppressing Cell Proliferation in Human Normal and Cancer Cells

We observed in Example 4, the metabolic activity (measured by WST-1) and morphology of A375 (melanoma) cells are sensitive to HC-HA/PTX3. As the concentration of HC-HA/PTX3 increased, metabolic activity was inhibited, and the cell morphology was changed from epithelial to spindle shape and then became round losing intercellular and cell-matrix adhesion. Since cell metabolic activity is directly proportional to the cell proliferation rate, the role of HC-HA/PTX3 in suppressing cell proliferation by quantifying DNA content of the proliferating cells will be determined. 5-bromo-2′-deoxyuridine (BrdU) incorporated into cellular DNA during cell proliferation will be detected using an anti-BrdU antibody by the BrdU Cell Proliferation Assay Kit (Cat #6813; Cell Signaling Technology, USA).

Considering the results in Example 4, wherein A375 cells showed significant morphological change starting from 25 μg/ml onwards of HC-HA/PTX3 after 24 hr treatment, cells were treated with HC-HA/PTX3 at following concentrations: 0, 25, 50 and 100 μg/ml for 24 hrs. 100 μg/ml of HA was used as control due to the lack of significant effect on cell morphology and metabolic activity as shown in Example 5. Based on this pilot study, the proliferation assay protocol was optimized and applied to assess the effect of HC-HA/PTX3 on human prostate cells (normal & cancer). Test groups are shown in Table 4.

A375 cells were seeded in 96-well plate (3200 cells in 100 μl culture medium/well) and were incubated for overnight. Cells were treated with HC-HA/PTX3 and HA at aforementioned concentrations for 48 hrs. 10 μl of 10×BrdU solution in each well and the cells were placed in incubator for 4 h. Removing medium, fixing/denaturing solution was added 100 μl/well for 30 min. Removing the solution, 1× detection antibody solution was added 100 μl/well for 1 h. The solution was removed and washed properly with wash buffer for three times and 1× HRP-conjugated secondary antibody solution was added 100 μl/well for 30 min at RT. Solution was removed and washed properly with wash buffer for three times and TMB substrate was added 100 μl/well for 30 min at RT. STOP solution was added 100 μL/well and absorbance was read at 450 nm.

TABLE 4
Experimental design - A375 cells

| | |
|---|---|
| | No. of |
| Sample | wells |

Cell line = A375
Unstimulated cells

| | |
|---|---|
| No treatment | 3 |
| No treatment + 1 × BrdU solution [BrdU control] | 3 |

Stimulated cells

| | |
|---|---|
| HA (100 μg/ml) for 24 h | 3 |
| HC-HA/PTX3 (25 μg/ml) for 24 h | 3 |
| HC-HA/PTX3 (50 μg/ml) for 24 h | 3 |
| HC-HA/PTX3 (100 μg/ml) for 24 h | 3 |

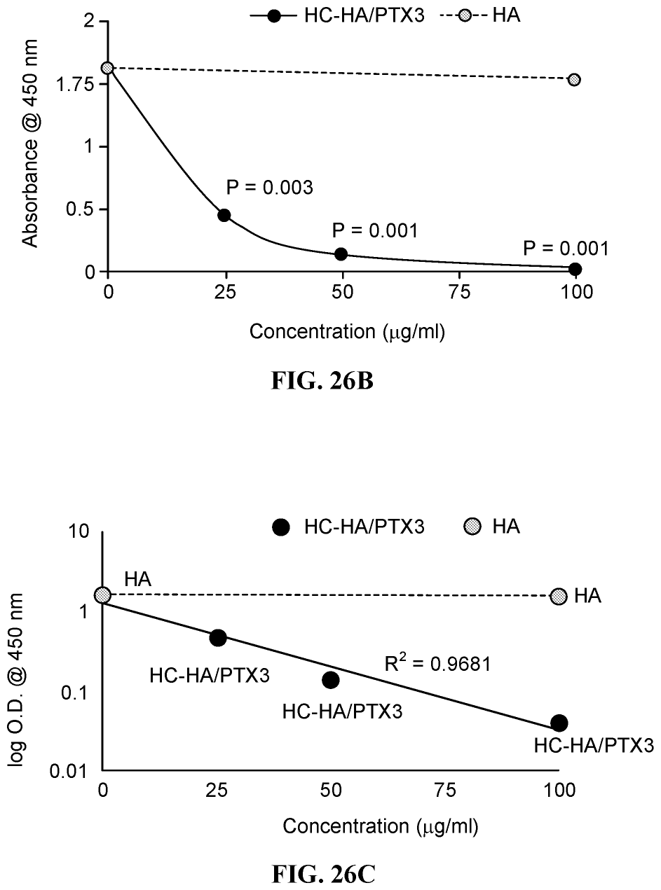

The anti-proliferative activity of HC-HA/PTX3 and HA was studied in A375 (melanoma) cells. 48 h incubation with different concentrations of HC-HA/PTX3 (25, 50 & 100 μg/ml) and 100 μg/ml HA showed same effect on A375 cell morphology as observed in Example 4 (FIG. 26A). BrdU incorporation assay reveals HC-HA/PTX3 inhibits A375 cell growth significantly in dose-dependent manner (p<0.005) while HA has no significant contribution (FIG. 26B). Statistical significance (p-value) calculated from Student's t-test. Semilog linear regression analysis determine that HC-HA/PTX3 has inhibitory effect on A375 cell growth in linear fashion (R²=0.9681). (FIG. 26C)

### Cell Proliferation Analysis of Prostate Cell Lines by BrdU Cell Proliferation Assay Kit (Cat #6813; Cell Signaling Technology, USA)

TABLE 5
Experimental Design - PrEC, PNT2, PC-3 & LNCaP cells

| | |
|---|---|
| | No. of |
| Sample | wells |

Cells or Cell-line = PrEC, PNT2, PC-3 & LNCaP
Unstimulated cells

| | |
|---|---|
| No treatment | 3 |
| No treatment + 1 × BrdU solution [BrdU control] | 3 |

Stimulated cells

| | |
|---|---|
| HA (100 μg/ml) for 48 h | 3 |
| HC-HA/PTX3 (1.56 μg/ml) for 48 h | 3 |
| HC-HA/PTX3 (3.13 μg/ml) for 48 h | 3 |
| HC-HA/PTX3 (6.25 μg/ml) for 48 h | 3 |
| HC-HA/PTX3 (12.5 μg/ml) for 48 h | 3 |
| HC-HA/PTX3 (25 μg/ml) for 48 h | 3 |

PrEC, PNT2, PC-3 and LNCaP cells were seeded at 3.2×10³ cells/well in a 96-well plate and incubated overnight. Cells were then treated with five concentrations of HC-HA/PTX3 (1.56, 3.13, 6.25, 12.5 & 25 μg/ml) and 100 μg/ml HMW-HA for 48 hrs in triplicate, as shown in Table 5. Finally, 10 μM BrdU was added to the well and cells were incubated for 4 hr. Medium was removed and fixing/ denaturing solution added 100 μl/well for 30 min. Solution was removed and 1× detection antibody solution was added and 1× HRP-conjugated secondary antibody solution was added 100 μl/well for 30 min at RT. Solution was removed and washed properly with wash buffer three times and TMB substrate was added 100 μl/well for 30 min at RT. STOP solution was added 100 μL/well and absorbance read at 450 nm.

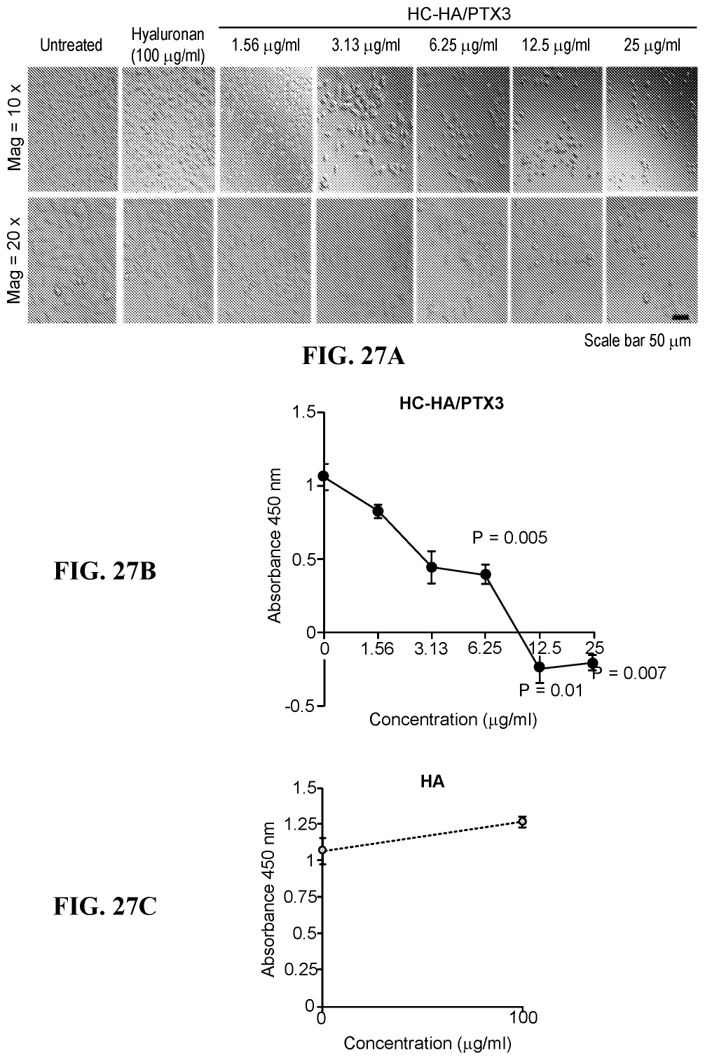

Cell morphology analysis of PrEC cells by bright-field phase contrast microscopy (FIG. 27A) and BrdU incorporation assay demonstrate an anti-proliferative effect of HC-HA/PTX3 on PrEC cells. HA does not inhibit cell proliferation. In this experiment, maximum concentration of HC-HA/PTX3 used was 25 μg/ml. At 12.5 and 25 μg/ml HC-HA/PTX3, the O.D. values became negative, indicating there were no cells in the wells. Due to a deviation in the analysis procedure these data may not be indicative of effects from HC-HA/PTX3 exposure.

FIG. 27A and 27B show HC-HA/PTX3 inhibited PrEC cell proliferation in a dose-dependent manner while HMW-HA (FIG. 27C) had no significant effect on cell proliferation as detected by BrdU Cell Proliferation Assay Kit #6813 (Cell Signaling, USA). FIG. 27A shows bright-field images of PrEC cell morphology in two magnifications (10× & 20×); FIGS. 27B and 27C show BrdU cell proliferation assay curve. Statistical significance (p value) calculated from Student's t-test.

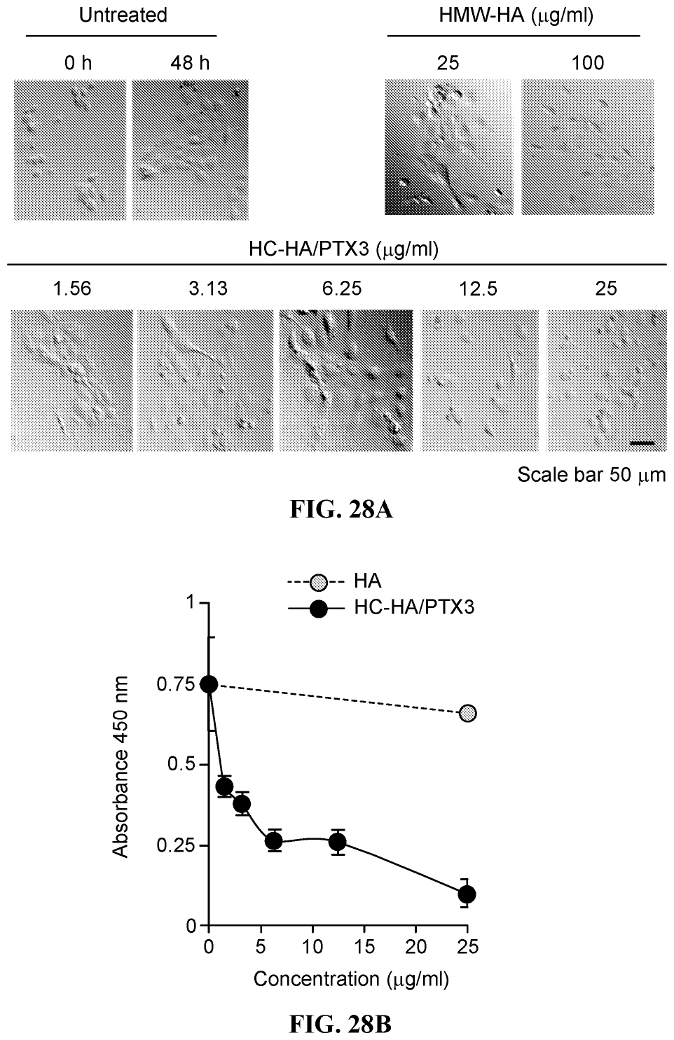

Cell morphology analysis by bright-field phase contrast microscopy and BrdU incorporation assay of PNT2 cells demonstrated anti-proliferative effect of HC-HA/PTX3 on PNT2 cells. HA did not inhibit cell proliferation. Normally cells grow in aggregate. As the concentration of HC-HA/PTX3 increased, it inhibited cell-cell adhesion (FIG. 28A). In BrdU assay, due to high standard error, a significant effect of HC-HA/PTX3 was not observed (FIG. 28B). P-value was more than 0.05. BrdU data showed similarity with earlier WST-1 data.

HC-HA/PTX3 inhibited PNT2 cell proliferation in a dose-dependent manner while HMW-HA had no significant effect on cell proliferation as detected by BrdU Cell Proliferation Assay Kit #6813 (Cell Signaling, USA). PNT2 cells were seeded at 3.2×10³ cells/well in a 96-well plate and incubated overnight. Cells were then treated with five concentrations of HC-HA/PTX3 (1.56, 3.13, 6.25, 12.5 & 25 μg/ml) and two concentrations of HMW-HA (25 & 100 μg/ml) for 48 hrs in triplicate. Finally, 10 μM BrdU was added to the well and cells were incubated for 4 hr.

HC-HA/PTX3, but not HA had anti-proliferative effect on PC-3 prostate cancer cell line as observed in Example 7. BrdU assay shows at 25 μg/ml concentration, HC-HA/PTX3 significantly inhibited proliferation of PC-3 (p=0.04). BrdU data showed similarity with the WST-1 data.

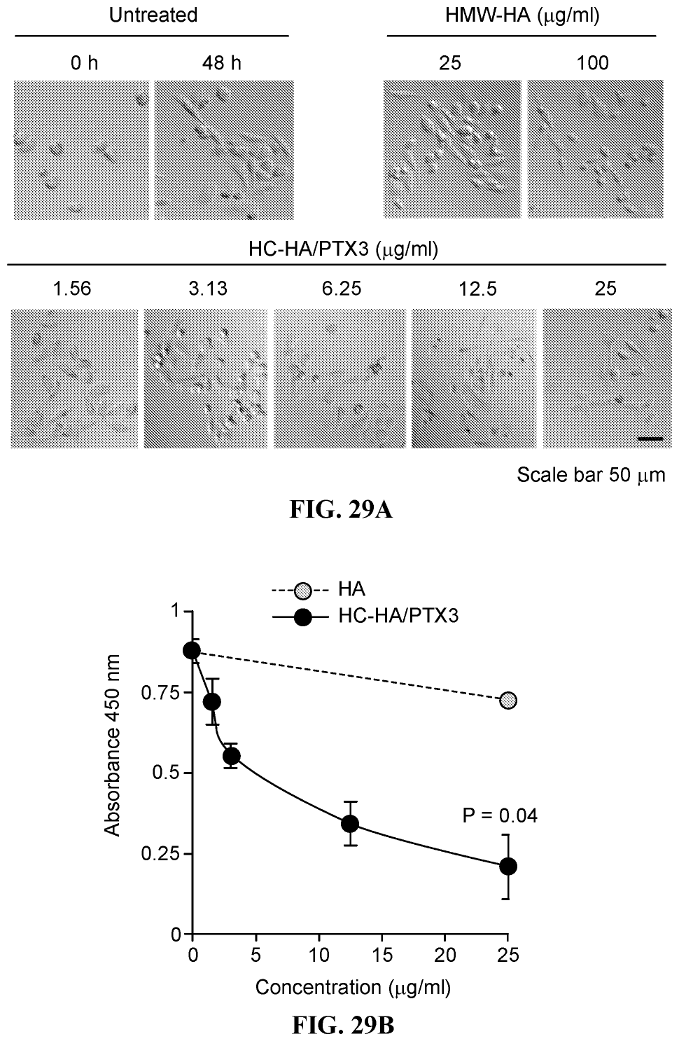

HC-HA/PTX3 inhibited PC3 cell proliferation in dose-dependent manner while HMW-HA had no significant effect on cell proliferation as detected by BrdU Cell Proliferation Assay Kit #6813 (Cell Signaling, USA). PC3 cells were seeded at 3.2×10³ cells/well in a 96-well plate and incubated overnight. Cells were then treated with five concentrations of HC-HA/PTX3 (1.56, 3.13, 6.25, 12.5 & 25 μg/ml) and two concentrations of HMW-HA (25 & 100 μg/ml) for 48 hrs in triplicate. Finally, 10 μM BrdU was added to the well and cells were incubated for 4 hr. FIG. 29A shows bright-field images of PC3 cell morphology. FIG. 29B shows BrdU cell proliferation assay curve.

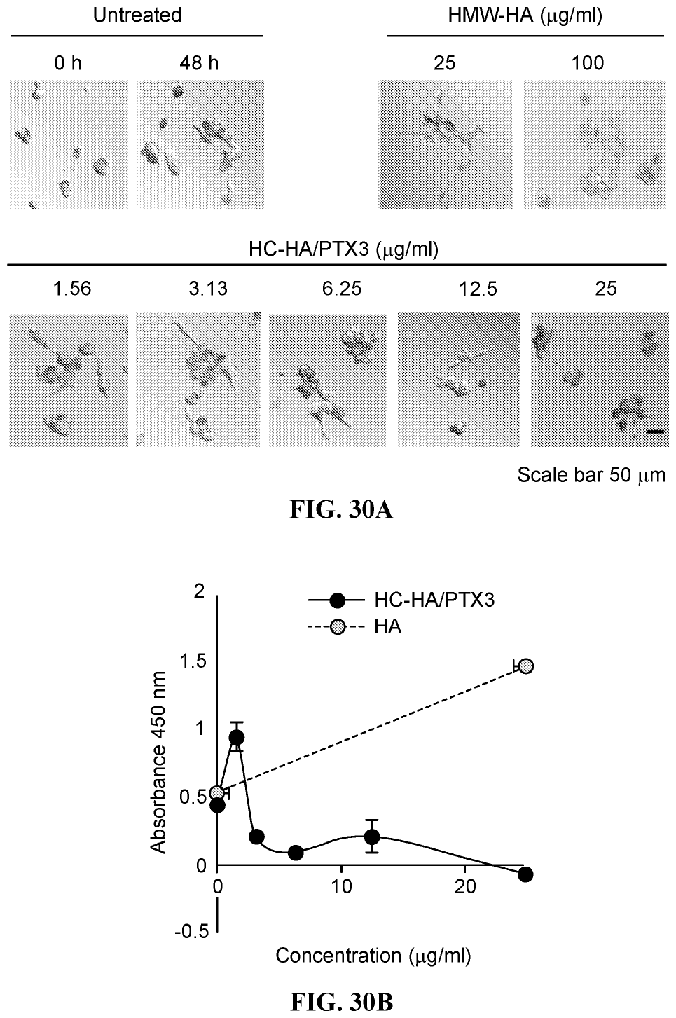

Under treatment with HC-HA/PTX3, the cell morphology of LNCaP was changed in a dose-dependent manner. Surprisingly, despite being a cancer cell, LNCaP did not proliferate as fast as PC-3 or PNT2 or PrEC cells. This observation was well corroborated in the BrdU data of all the cell types. Under untreated condition, O.D. values of LNCaP cells came 0.5 while for rest other cells it was >0.75. Interestingly, for LNCaP, cells kept on growing in aggregate even at high concentration of HC-HA/PTX3, but their morphology changed, thus implicating, the effect of HC-HA/PTX3 more on cell-matrix attachment rather than cell-cell adhesion. High standard error at concentration 3.13 and 12.5 μg/ml of HC-HA/PTX3 and at 100 μg/ml HA, represented wrong graph. O.D. value was negative at 25 μg/ml HC-HA/PTX3 due to a pipetting error in the blank sample (See, FIG. 30A). Sufficient cells could be visualized in the subject well (See, FIG. 30B).

HC-HA/PTX3 inhibited LNCaP cell proliferation in dose-dependent manner while HMW-HA had no significant effect on cell proliferation as detected by BrdU Cell Proliferation Assay Kit #6813 (Cell Signaling, USA). LNCaP cells were seeded at 3.2×10³ cells/well in a 96-well plate and incubated overnight. Cells were then treated with five concentrations of HC-HA/PTX3 (1.56, 3.13, 6.25, 12.5 & 25 μg/ml) and two concentrations of HMW-HA (25 & 100 μg/ml) for 48 hrs in triplicate. Finally, 10 μM BrdU was added to the well and cells were incubated for 4 hr. FIG. 30A shows bright-field images of LNCaP cell morphology. FIG. 30B shows BrdU cell proliferation assay curve.

BrdU assay on four types of prostate epithelial cells/cell lines (both normal and cancer) suggested that HC-HA/PTX3, but not HMW-HA, inhibited cell proliferation in a dose-dependent manner. Because of technical errors, results of PrEC and LNCaP do not reflect same impression as observed in WST-1 assay data. However, the corresponding cell morphology images supported that the effect of HC-HA/PTX3 and HA on the prostate cells were similar as observed in previous experiments.

While preferred embodiments have been shown and described herein, it will be obvious to those skilled in the art that such embodiments are provided by way of example only. Numerous variations, changes, and substitutions may now occur. It should be understood that various alternatives to the embodiments described herein can be employed in practicing the described methods. It is intended that the following claims define the scope of the embodiments and that methods and structures within the scope of these claims and their equivalents be covered thereby.

What is claimed is:

1. A method of inhibiting cancer cell regrowth of a tumor in an individual in need thereof, comprising contacting an area surrounding the tumor after a surgical procedure with an isolated heavy chain-hyaluronan/pentraxin 3 (HC-HA/PTX3) complex, thereby inhibiting cancer cell regrowth at the area surrounding the tumor.

2. The method of claim 1, wherein the surgical procedure comprises surgical excision, cryoablation, or radiofrequency ablation of the tumor.

3. The method of claim 1, wherein the surgical procedure comprises chemotherapy, immunotherapy, or targeted therapy.

4. The method of claim 1, wherein the area surrounding the tumor comprises a surgical margin.

5. The method of claim 1, wherein the area surrounding the tumor is a peritumor region.

6. The method of claim 1, wherein the tumor is a solid tumor.

7. The method of claim 1, wherein the tumor is a cancer selected from the group consisting of liver cancer, pancreatic cancer, bladder cancer, prostate cancer, lung cancer, non-small cell lung carcinoma, ovarian cancer, breast cancer, melanoma, gastric cancer, colon cancer, colorectal cancer, central nervous system (CNS) cancer, bone cancer, a lymphoma, skin cancer, head and neck cancer, kidney cancer, testicular cancer, uterine cancer, cervical cancer, esophageal cancer, thyroid cancer, salivary gland cancer, adrenal cancer, and gastrointestinal cancer.

8. The method of claim 7, wherein the cancer is an inoperable cancer.

9. The method of claim 7, wherein the cancer is pancreatic cancer.

10. The method of claim 7, wherein the cancer is prostate cancer.

11. The method of claim 7, wherein the cancer is glioblastoma multiforme.

12. The method of claim 7, wherein the cancer is skin cancer.

13. The method of claim 7, wherein the cancer is colon cancer.

14. The method of claim 7, wherein the cancer is lung cancer.

15. The method of claim 7, wherein the cancer is breast cancer.

16. The method of claim 1, wherein the area surrounding the tumor is contacted with about 10 microgram to 100 milligrams.

17. The method of any one of claims 1 to 16, wherein the HC-HA/PTX3 complex is native HC-HA/PTX3 complex, reconstituted HC-HA/PTX3 complex, or a combination thereof.

18. The method of claim 17, wherein the native HC-HA/PTX3 complex is isolated from a fetal support tissue.

19. The method of claim 17, wherein the reconstituted HC-HA/PTX3 complex comprises heavy chain 1 (HC1) and heavy chain 2 (HC2) of inter-α-inhibitor (IαI), hyaluronic acid (HA), and PTX3.

20. The method of claim 17, wherein the reconstituted HC-HA/PTX3 complex comprises HC1, HC2, HA, PTX3 and tumor necrosis factor α-stimulated gene 6 (TSG-6).

21. The method of claim 1, wherein the hyaluronan (HA) is high molecular weight hyaluronan (HMW HA).

22. The method of claim 1, wherein the hyaluronan (HA) is low molecular weight hyaluronan (LMW HA).

23. The method of claim 1, wherein the HC-HA/PTX3 complex is cryopreserved.

24. The method of claim 1, wherein the HC-HA/PTX3 complex comprises viable cells.

25. The method of claim 1, further comprising administering a therapeutic agent.

26. The method of claim 25, wherein the therapeutic agent is selected from the group consisting of a chemotherapeutic, an analgesic, an anti-inflammatory, a steroid, an immunotherapy, cellular therapy, a radiotherapy, a targeted drug therapeutic, and an antibiotic.

27. The method of claim 25, wherein administering the therapeutic agent occurs before contacting the area surrounding the tumor with the HC-HA/PTX3 complex.

28. The method of claim 25, wherein administering the therapeutic agent occurs after contacting the area surrounding the tumor with the HC-HA/PTX3 complex.

29. The method of claim 25, wherein administering the therapeutic agent occurs concurrently with contacting the area surrounding the tumor with the HC-HA/PTX3 complex.

30. The method of any one of claims 1 to 29, wherein the method inhibits tumor cell regrowth by killing cancer cells.

31. The method of claim 30, wherein the killing of the cancer cells is by apoptosis or necrosis.

32. The method of any one of claims 1 to 29, wherein the method inhibits tumor cell regrowth by inhibiting proliferation of cancer cells.

33. The method of any one of claims 1 to 29, wherein the method inhibits tumor cell regrowth by inhibiting metabolic activity of cancer cells.

34. A method of killing cancer cells of a tumor in an individual in need thereof, comprising contacting a tumor or an area surrounding the tumor prior to, during or after a surgical procedure with an isolated heavy chain-hyaluronan/pentraxin 3 (HC-HA/PTX3) complex, thereby killing the cancer cells.

35. The method of claim 34, wherein the surgical procedure comprises surgical excision, cryoablation, or radiofrequency ablation of the tumor.

36. The method of claim 34, wherein the surgical procedure comprises chemotherapy, immunotherapy, or targeted therapy.

37. The method of claim 34, wherein the area surrounding the tumor comprises a surgical margin.

38. The method of claim 34, wherein the area surrounding the tumor is a peritumor region.

39. The method of claim 34, wherein the tumor is a solid tumor.

40. The method of claim 34, wherein the tumor is a cancer selected from the group consisting of liver cancer, pancreatic cancer, bladder cancer, prostate cancer, lung cancer, non-small cell lung carcinoma, ovarian cancer, breast cancer, melanoma, gastric cancer, colon cancer, colorectal cancer, central nervous system (CNS) cancer, bone cancer, a lymphoma, skin cancer, head and neck cancer, kidney cancer, testicular cancer, uterine cancer, cervical cancer, esophageal cancer, thyroid cancer, salivary gland cancer, adrenal cancer, and gastrointestinal cancer.

41. The method of claim 34, wherein the cancer is an inoperable cancer.

42. The method of claim 34, wherein the cancer is pancreatic cancer.

43. The method of claim 34, wherein the cancer is prostate cancer.

44. The method of claim 34, wherein the cancer is glioblastoma multiforme.

45. The method of claim 34, wherein the cancer is skin cancer.

46. The method of claim 34, wherein the cancer is colon cancer.

47. The method of claim 34, wherein the cancer is lung cancer.

48. The method of claim 34, wherein the cancer is breast cancer.

49. The method of claim 34, wherein the area surrounding the tumor is contacted with about 10 microgram to 100 milligrams.

50. The method of any one of claims 34 to 49, wherein the HC-HA/PTX3 complex is native HC-HA/PTX3 complex, reconstituted HC-HA/PTX3 complex, or a combination thereof.

51. The method of claim 50, wherein the native HC-HA/PTX3 complex is isolated from a fetal support tissue.

52. The method of claim 50, wherein the reconstituted HC-HA/PTX3 complex comprises heavy chain 1 (HC1) and heavy chain 2 (HC2) of inter-α-inhibitor (IαI), hyaluronic acid (HA), and PTX3.

53. The method of claim 50, wherein the reconstituted HC-HA/PTX3 complex comprises HC1, HC2, HA, PTX3 and tumor necrosis factor α-stimulated gene 6 (TSG-6).

54. The method of claim 34, wherein the HC-HA/PTX3 complex is cryopreserved.

55. The method of claim 34, wherein the HC-HA/PTX3 complex comprises viable cells.

56. The method of claim 34, wherein the hyaluronan (HA) is high molecular weight hyaluronan (HMW HA).

57. The method of claim 34, wherein the hyaluronan (HA) is low molecular weight hyaluronan (LMW HA).

58. The method of claim 34, wherein the contacting comprises injecting the HC-HA/PTX3 directly into a tumor.

59. The method of claim 34, further comprising administering a therapeutic agent.

60. The method of claim 59, wherein the therapeutic agent is selected from the group consisting of a chemotherapeutic, an analgesic, an anti-inflammatory, a steroid, an immunotherapy, cellular therapy, a radiotherapy, a targeted drug therapeutic, and an antibiotic.

61. The method of claim 59, wherein administering the therapeutic agent occurs before contacting the cancer cells with the HC-HA/PTX3 complex.

62. The method of claim 59, wherein administering the therapeutic agent occurs after contacting the cancer cells with the HC-HA/PTX3 complex.

63. The method of claim 59, wherein administering the therapeutic agent occurs concurrently with contacting the cancer cells with the HC-HA/PTX3 complex.

64. The method of claim 34, wherein the killing of the cancer cells is by apoptosis or necrosis.
